(19) United States
(12) Patent Application Publication
(10) Pub. No.: US 2023/0392200 A1
(43) Pub. Date: Dec. 7, 2023

(54) COMPOSITIONS AND METHODS FOR GENERATING RECOMBINANT ANTIGEN BINDING MOLECULES FROM SINGLE CELLS

(71) Applicant: 10x Genomics, Inc., Pleasanton, CA (US)

(72) Inventors: Michael John Terry STUBBINGTON, Cambridge (GB); Wyatt James McDONNELL, Concord, CA (US)

(21) Appl. No.: 18/299,593

(22) Filed: Apr. 12, 2023

Related U.S. Application Data
(63) Continuation of application No. PCT/US2021/054690, filed on Oct. 13, 2021.
(60) Provisional application No. 63/175,022, filed on Apr. 14, 2021.
(60) Provisional application No. 63/091,196, filed on Oct. 13, 2020.

(51) Int. Cl. C12Q 1/6869; G16B 15/30

(52) U.S. Cl. CPC C12Q 1/6869; G16B 15/30

(57) ABSTRACT

The present disclosure relates generally to the field of immunology, and particularly relates to compositions, methods, and systems for the analysis and generation of antigen-binding molecules produced by immune cells obtained from tumor samples (e.g., antibodies produced by B cells obtained from tumor samples or TCRs produced by T cells obtained from tumor samples), using single-cell immune profiling methodologies, so as to produce recombinant antigen-binding molecules (e.g., antibodies, TCRs) with desired properties.

### CROSS-REFERENCE TO RELATED APPLICATIONS

This application is a continuation application of International Patent Application No. PCT/US2021/054690, filed on Oct. 13, 2021; which claims the benefit of priority to U.S. Provisional Patent Application Ser. No. 63/091,196, filed on Oct. 13, 2020, and U.S. Provisional Patent Application Ser. No. 63/175,022, filed on Apr. 14, 2021. The disclosures of the above-referenced applications are herein expressly incorporated by reference it their entireties, including any drawings.

### FIELD

The present disclosure relates generally to the field of immunology, and particularly relates to compositions, methods, and systems for the analysis and generation of antigen-binding molecules produced by immune cells obtained from tumor samples (e.g., antibodies produced by B cells obtained from tumor samples or TCRs produced by T cells obtained from tumor samples), using single-cell immune profiling methodologies, so as to produce recombinant antigen-binding molecules (e.g., antibodies, TCRs) with desired properties.

### INCORPORATION OF THE SEQUENCE LISTING

This application contains a Sequence Listing which is hereby incorporated by reference in its entirety. The accompanying Sequence Listing text file, named “057862-522C01US_Sequence_Listing_ST25.txt,” was created on Apr. 4, 2023 and is 5 KB.

### BACKGROUND

The identification and evaluation of therapeutic proteins, especially antigen-binding molecules, e.g., therapeutic antibodies and TCRs is a core strategy for a number of pharmaceutical and biotechnology companies. For example, antibody-based therapy has become established over the past 15 years and is currently one of the most successful and important strategies for treating patients with hematological malignancies and solid tumors. In particular, the use of monoclonal antibodies (mAbs) for cancer therapy has achieved considerable success in recent years in the field of pharmaceutical biotechnology. Several monoclonal antibodies (mAbs) have been identified for use as therapeutic compounds in the treatment of various types of health condition and diseases. There are to date more than 45 mAbs marketed in various fields such as oncology, immunology, ophthalmology and cardiology. In particular, monoclonal antibodies have provided important medical results in the treatment of several major diseases including autoimmune, cardiovascular and infectious diseases, cancer and inflammation, clinical trials.

A number of processes and systems are currently available for the isolation and characterization of antigen-binding molecules (e.g., monoclonal antibodies), including hybridoma capture, phage display of human antibody libraries, yeast display of antibody libraries, and direct capture. Similarly, several approaches and techniques have been developed for the isolation of circulating tumor cells (CTCs) using antibody capture, microfluidics, and combinations thereof. However, it has been reported that rapid isolation of cancer-specific antibodies, patient-specific antibodies, potentially inter-patient cancer-specific antibodies, and direct screening of these isolated antibodies for tumor-specificity poses multiple challenges. Limitations of current approaches include, e.g., (i) a lack of heavy-light chain pairing (bulk approaches), (ii) inability to efficiently amplify B cell receptor sequences due to poor RNA quality or sample preparation conditions, (iii) low-throughput due to inability to combine and analyze samples from multiple individuals, or such low input that single cell analysis is not possible, and (iv) generation of antibodies that are not fully humanized (e.g., (e.g. “humanized” VDJ mice which still require additional humanization), unlike those antibodies found natively in tumors.

Therefore, there is a need for alternative approaches that couple high-throughput single-cell phenotypic screening with high-throughput sequencing of antibody-producing primary cells that produce antigen-binding molecules, such as B cells and T cells, in a flexible format that enables direct screening for functional activities.

### SUMMARY

The present disclosure provides, inter alia, compositions and methods for the analysis and generation of antibodies produced by B cells obtained from tumor samples, using single-cell immune profiling methodologies, so as to produce recombinant antibodies with desired properties. The present disclosure thus can be useful for various downstream applications, including identification and/or isolation of antibodies having specificity for a tumor, for an individual, or for population of individuals. Also provided in some embodiments of the disclosure are recombinant antibodies, compositions and methods useful for the production of such antibodies, as well as kits and systems for antibody discovery and/or management.

In one aspect of the disclosure, provided herein are methods for generating a recombinant antibody, the methods including: (a) partitioning a single B cell of a plurality of single B cells obtained from a first tumor sample in a partition of a first plurality of partitions; (b) determining all or a part of the nucleic acid sequences encoding one or more antibodies produced by the partitioned single B cell; and (c) using the determined nucleic acid sequences to produce a recombinant antibody. Non-limiting exemplary embodiments of the methods of the disclosure can include one or more of the following features. In some embodiments, a method of the disclosure further includes including coupling a reporter oligonucleotide including a reporter barcode sequence to the recombinant antibody to generate a barcoded recombinant antibody. In some embodiments, the reporter barcode sequence of the reporter oligonucleotide includes one or more unique identifiers for the recombinant antibody. In some embodiments, at least one the one or more unique identifiers for the recombinant is a partition-specific barcode sequence. In some embodiments, the method of the disclosure further including determining all or a part of the nucleic acid identifier to identify the barcoded recombinant antibody. In some embodiments, the reporter oligonucleotide comprise an adapter region that allows for downstream analysis of the recombinant antibody. In some embodiments, the adapter region comprises a primer binding site and/or a cleavage site. In some embodiments, the methods further include partitioning one or more nucleic acid barcode molecules into the partition; the one or more nucleic acid barcode molecules comprising a common barcode sequence. In some embodiments, the methods further include partitioning cell lysis reagents into the partition in order to release the contents of the partitioned single B cell. In some embodiments, the methods further include using the one or more nucleic acid barcode molecules and one or more nucleic acid analytes of the partitioned B single cell to generate one or more barcoded nucleic acid molecule comprising a coding sequence for an antibody produced by the partitioned B single cell, or any fragments thereof. In some embodiments, the method of the disclosure further including contacting the barcoded recombinant antibody to a single tumor cell obtained from a second tumor sample. In some embodiments, the method further including identifying the recombinant antibody as a tumor specific antibody (e.g., identifying the recombinant antibody as an antibody having specificity for the second tumor sample) if the barcoded recombinant antibody is capable of binding to an antigen associated with the second tumor sample.

In some embodiments, the first and/or the second tumor sample is derived from solid tumor, a soft tissue tumor, a metastatic lesion, a non-solid tumor, a circulating tumor cell (CTC) population, a tumor cell line, and a patient derived xenograft (PDX). In some embodiments, the first and the second tumor samples are derived from the same subject. In some embodiments, the first and the second tumor samples are derived from the same tumor.

In some embodiments, the method of the disclosure further includes, prior to (a), dissociating the first tumor sample to generate a population of single cells. In some embodiments, the method further includes isolating and/or enriching the plurality of single B cells prior to (a).

In some embodiments, the method further includes including dissociating the second tumor sample to generate a population of single cells prior to contacting the barcoded recombinant antibody to a single tumor cell of the generated single cell population. In some embodiments, the methods further includes isolating and/or enriching the single cell tumor cell prior to contacting the barcoded recombinant antibody to a single tumor cell. In some embodiments, the single tumor cell is a circulating tumor cell (CTC).

In some embodiments, step (a) of the method disclosed herein includes individually partitioning additional single B cells of the plurality of B cells in partitions of the first plurality of partitions, and step (b) further includes determining all or a part of the nucleic acid sequences encoding antibodies produced by the additional single B cells. In some embodiments, the method further includes individually partitioning one or more single tumor cells from the second tumor sample in a partition of a second plurality of partition. In some embodiments, at least one of the first and second plurality of partitions includes a microwell or a droplet. In some embodiments, the reporter oligonucleotides contained within a partition are distinguishable from the reporter oligonucleotides contained within other partitions of the plurality of partitions.

In some embodiments, the method of the disclosure includes contacting the single tumor cell obtained from the second tumor sample with a composition including one or more of the following: (i) one or more barcoded immune-cell marker antibodies and/or barcoded tumor-cell marker antibodies; (ii) one or more barcoded therapeutic antibodies; and (iii) the barcoded recombinant antibody identified as having specificity for the second tumor sample.

In some embodiments, the one or more therapeutic antibodies is selected from the group consisting of abciximab, adalimumab, aducanumab, alacizumab, alemtuzumab, alirocumab, alirocumab, ascrinvacumab, atezolizumab, atinumab, bapineuzumab, basiliximab, basiliximab, belimumab, bevacizumab, blinatumomab, blosozumab, bococizumab, brentuximab, canakinumab, caplacizumab, capromab, certolizumab, cetuximab, crenezumab, daclizumab, daratumumab, demcizumab, denosumab, denosumab, dinutuximab, ecukinumab, eculizumab, eculizumab, efalizumab, elotuzumab, enoticumab, etaracizumab, evinacumab, evolocumab, evolocumab, fasinumab, fulranumab, gantenerumab, golimumab, ibritumomab, icrucumab, idarucizumab, idarucizumab, inciacumab, infliximab, ipilimumab, mepolizumab, natalizumab, necitumumab, nesvacumab, nivolumab, obinutuzumab, ofatumumab, omalizumab, opicinumab, orticumab, ozanezumab, palivizumab, palivizumab, panitumumab, pembrolizumab, pertuzumab, ponezumab, ralpancizumab, ramucirumab, ramucirumab, ranibizumab, raxibacumab, refanezumab, rinucumab, rituximab, romosozumab, siltuximab, solanezumab, stamulumab, tadocizumab, tanezumab, tocilizumab, trastuzumab, ustekinumab, vedolizumab, and vesencumab.

In some embodiments, the one or more immune-cell marker antibodies is selected from the group consisting of antibodies having specificity for B cells, T cells, monocytes, macrophages, granulocytes (basophil, eosinophil, neutrophil), dendritic cells, natural killer (NK) cells, and natural killer T (NKT) cells.

In some embodiments, the one or more tumor-cell marker antibodies is selected from the group consisting of antibodies having specificity for ALK, alpha-fetoprotein (AFP), beta-2-microglobulin (B2M), beta-human chorionic gonadotropin (Beta-hCG), bladder tumor antigen (BTA), BRCA1, BRCA2, BCR-ABL fusion gene (Philadelphia chromosome), BRAF V600 mutations, C-kit/CD117, CA15-3/CA27.29, CA-125, CA 27.29, carcinoembryonic antigen (CEA), CD20, CD22, CD25, CD30, CD31, CD33, CD44, CD133, CD176, CD276, estrogen receptor (ER), E-cadherin, ESPR, EGFR, EPCAM, GD2, progesterone receptor (PR), fibrin/fibrinogen, HE4 gene variants, HER2 gene variants, JAK2 gene variants, KRAS gene variants, nuclear matrix protein 22, PCA3, PML/RARα fusion gene, programmed death-ligand 1 (PD-L1 or CD274), prostate-specific antigen (PSA), TEM7, TEM8, and VEGF receptor family members.

In one aspect, provided herein are methods for identifying a tumor-specific antibody, the method including: (a) partitioning a single B cell of a plurality of single B cells obtained from a first tumor sample in a partition of a first plurality of partitions; (b) determining all or a part of the nucleic acid sequences encoding one or more antibodies produced by the partitioned single B cell; (c) using the determined nucleic acid sequences to produce a recombinant antibody; (d) coupling the recombinant antibody to a reporter oligonucleotide including a reporter barcode sequence to generate a barcoded recombinant antibody; and (e) contacting the barcoded recombinant antibody with a second tumor sample, and identifying the recombinant antibody as an antibody having specificity for the second tumor sample if the barcoded recombinant antibody is capable of binding to an antigen associated with the tumor sample.

Non-limiting exemplary embodiments of the methods for identifying a tumor-specific antibody as described herein can include one or more of the following features. In some embodiments, the reporter barcode sequence of the reporter oligonucleotide includes one or more unique identifiers for the recombinant antibody. In some embodiments, the methods further include determining all or a part of the nucleic acid sequence of the reporter oligonucleotide to identify the barcoded recombinant antibody. In some embodiments, the reporter oligonucleotide include an adapter region that allows for downstream analysis of the recombinant antibody. In some embodiments, the adapter region includes a primer binding site and/or a cleavage site. In some embodiments, step (a) further includes partitioning cell lysis reagents into the partition in order to release the contents of the partitioned single B cell. In some embodiments, step (a) further includes partitioning one or more nucleic acid barcode molecules into the partition; the one or more nucleic acid barcode molecules including a common barcode sequence. In some embodiments, the methods further include using the one or more nucleic acid barcode molecules and one or more nucleic acid analytes of the partitioned B single cell to generate one or more barcoded nucleic acid molecule including a coding sequence for an antibody produced by the partitioned B single cell, or any fragments thereof.

In some embodiments, the first and/or the second tumor sample is derived from a solid tumor, a soft tissue tumor, a metastatic lesion, a non-solid tumor, a circulating tumor cell (CTC) population, a tumor cell line, and a patient derived xenograft (PDX). In some embodiments, the first and the second tumor samples are derived from the same subject. In some embodiments, the first and the second tumor samples are derived from the same tumor. In some embodiments, the methods further include, prior to (a), dissociating the first tumor sample to generate a population of single cells. In some embodiments, the methods further include isolating and/or enriching the plurality of single B cells prior to (a). In some embodiments, the methods further include dissociating the second tumor sample to generate a population of single cells prior to contacting the barcoded recombinant antibody to a single tumor cell of the generated single cell population. In some embodiments, the methods further include isolating and/or enriching the single cell tumor cell prior to contacting the barcoded recombinant antibody to a single tumor cell. In some embodiments, the single tumor cell is a circulating tumor cell (CTC). In some embodiments, step (e) further comprises contacting the barcoded recombinant antibody with a control sample. In some embodiments, the control sample is (i) a non-tumor sample or (ii) a sample that the barcoded recombinant antibody is not expected to bind.

In some embodiments, step (a) further includes individually partitioning additional single B cells of the plurality of B cells in partitions of the first plurality of partitions, and step (b) further includes determining all or a part of the nucleic acid sequences encoding antibodies produced by the additional single B cells. In some embodiments, the methods further include individually partitioning one or more single tumor cells from the second tumor sample in a partition of a second plurality of partition. In some embodiments, at least one of the first and second plurality of partitions includes a microwell or a droplet. In some embodiments, the reporter oligonucleotides contained within a partition are distinguishable from the reporter oligonucleotides contained within other partitions of the plurality of partitions.

In some embodiments, the methods include contacting the single tumor cell obtained from the second tumor sample with a composition including one or more of the following: (i) one or more barcoded immune-cell marker antibodies and/or barcoded tumor-cell marker antibodies; (ii) one or more barcoded therapeutic antibodies; and (iii) the barcoded recombinant antibody identified as having specificity for the second tumor sample. In some embodiments, the one or more therapeutic antibodies is selected from the group consisting of abciximab, abciximab, adalimumab, aducanumab, alacizumab, alemtuzumab, alirocumab, alirocumab, ascrinvacumab, atezolizumab, atinumab, bapineuzumab, basiliximab, basiliximab, belimumab, bevacizumab, blinatumomab, blosozumab, bococizumab, brentuximab, canakinumab, caplacizumab, capromab, certolizumab, cetuximab, crenezumab, daclizumab, daratumumab, demcizumab, denosumab, denosumab, dinutuximab, ecukinumab, eculizumab, eculizumab, efalizumab, elotuzumab, enoticumab, etaracizumab, evinacumab, evolocumab, evolocumab, fasinumab, fulranumab, gantenerumab, golimumab, ibritumomab, icrucumab, idarucizumab, idarucizumab, inciacumab, infliximab, ipilimumab, mepolizumab, natalizumab, necitumumab, nesvacumab, nivolumab, obinutuzumab, ofatumumab, omalizumab, opicinumab, orticumab, ozanezumab, palivizumab, palivizumab, panitumumab, pembrolizumab, pertuzumab, ponezumab, ralpancizumab, ramucirumab, ramucirumab, ranibizumab, raxibacumab, refanezumab, rinucumab, rituximab, romosozumab, siltuximab, solanezumab, stamulumab, tadocizumab, tanezumab, tocilizumab, trastuzumab, ustekinumab, vedolizumab, and vesencumab. In some embodiments, the one or more immune-cell marker antibodies is selected from the group consisting of antibodies having specificity for one or more of B cells, T cells, monocytes, macrophages, granulocytes (basophil, eosinophil, neutrophil), dendritic cells, NK cells, and NKT cells. In some embodiments, the one or more tumor-cell marker antibodies is selected from the group consisting of antibodies having specificity for ALK, alpha-fetoprotein (AFP), beta-2-microglobulin (B2M), beta-human chorionic gonadotropin (Beta-hCG), bladder tumor antigen (BTA), BRCA1, BRCA2, BCR-ABL fusion gene (Philadelphia chromosome), BRAF V600 mutations, C-kit/CD117, CA15-3/CA27.29, CA-125, CA 27.29, carcinoembryonic antigen (CEA), CD20, CD22, CD25, CD30, CD31, CD33, CD44, CD133, CD176, CD276, estrogen receptor (ER), E-cadherin, ESPR, EGFR, EPCAM, GD2, progesterone receptor (PR), fibrin/fibrinogen, HE4 gene variants, HER2 gene variants, JAK2 gene variants, KRAS gene variants, nuclear matrix protein 22, PCA3, PML/RARα fusion gene, programmed death-ligand 1 (PD-L1 or CD274), prostate-specific antigen (PSA), TEM7, TEM8, and VEGF receptor family members.

In some embodiments, the identifying of the produced antibody as an antibody having specificity for the second tumor sample further including quantifying levels of gene expression and protein marker expression in the single tumor cell. In some embodiments, the method further includes using the quantified levels for identification of biomarkers specific for the second tumor sample and/or a subject from whom the second tumor sample is obtained. In some embodiments, the method further includes quantifying binding affinity of the one or more therapeutic antibodies to the single tumor cell. In some embodiments, the method further includes using the quantified binding affinity as an indicator of efficacy of treating a tumor with the one or more therapeutic antibodies. In some embodiments, the method further includes using the quantified binding affinity to monitor antigen escape of a tumor from the one or more therapeutic antibodies over time.

In some embodiments, the identifying of the produced antibody as an antibody having specificity for the second tumor sample further including comparing the determined nucleic acid sequences encoding the antibody to a genomic DNA sequence from the second tumor sample to confirm antigen specificity of the antibody. In some embodiments, the genomic DNA sequence is obtained from a single cell in the second tumor sample. In some embodiments, the genomic DNA sequence is obtained from a plurality of cells in the second tumor sample. In some embodiments, the genomic DNA sequence is obtained by whole-genome sequencing.

In some embodiments, the identifying of the produced antibody as an antibody having specificity for the second tumor sample further includes comparing the determined nucleic acid sequences encoding the barcoded antibody to a sequence of a ribonucleic acid (RNA) molecule from the second tumor sample to confirm antigen specificity of the antibody. In some embodiments, the RNA molecule is obtained from a single cell in the second tumor sample. In some embodiments, the RNA molecule is obtained from a plurality of cells in the second tumor sample. In some embodiments, the method further includes obtaining the sequence of the RNA molecule. In some embodiments, the method further includes determining a nucleic acid sequence of a messenger RNA (mRNA) from the single B cell and/or from the single tumor cell. In some embodiments, the determining includes binding one or more primers to the mRNA and optionally generating a complementary DNA (cDNA) via reverse transcription. In some embodiments, the one or more nucleic acid barcode molecules independently include one or more barcode sequences. In some embodiments, the one or more barcode sequences is selected from the group consisting of a sample barcode, a tissue barcode, a cell barcode, a spatial barcode, a partition-specific barcode, and a unique molecular identifier (UMI). In some embodiments, the one or more primers are coupled to a microcapsule. In some embodiments, the microcapsule comprises a bead. In some embodiments, the determining includes whole transcriptome sequencing, e.g., whole-exome sequencing. In some embodiments, the determining includes next-generation sequencing (NGS).

In some embodiments, the method further includes generating a chimeric antigen receptor using the nucleic acid sequence of the recombinant antibody. In some embodiments, the method further includes administering the recombinant antibody to a subject in need thereof. In some embodiments, the method further includes administering an immune cell expressing the recombinant antibody to a subject in needed thereof. In some embodiments, the method further includes comparing the determined nucleic acid sequence of the recombinant antibody to sequences of known antibodies in order to identify the antibody as a tumor-specific antibody. In some embodiments, the method further includes using a filter that takes into account clonal expansions to identify the antibody as a tumor-specific antibody. In some embodiments, the method further includes using a filter that takes into account somatic hypermutation and isotype usage to identify the antibody as a tumor-specific antibody. In some embodiments, the method further includes using a filter that takes into account gene expression profiles of the single B cell to identify the antibody as a tumor-specific antibody.

In another aspect, provided herein are recombinant antibodies or a functional fragment thereof generated or identified by a method as described herein.

In another aspect, provided herein are recombinant nucleic acids including a nucleic acid sequence that encode the recombinant antibody of the disclosure or a functional fragment thereof. In some embodiments, the recombinant nucleic acid is further configured as an expression cassette in a vector. In some embodiments, the vector is a plasmid vector or a viral vector.

In yet another aspect, provided herein are recombinant cells that include a recombinant nucleic acid of the disclosure. In some embodiments, the recombinant cell is a prokaryotic cell or a eukaryotic cell.

In one aspect, provided herein are compositions including one or more of the following: (a) a recombinant antibody of the disclosure; (b) a recombinant nucleic acid of the disclosure; (c) a recombinant cell of the disclosure; (d) one or more barcoded immune-cell marker antibodies and/or barcoded tumor-cell marker antibodies; (e) one or more barcoded therapeutic antibodies; and (f) the barcoded recombinant antibody identified in claim 11 as having specificity for the second tumor sample. In some embodiments, the compositions include a pharmaceutically acceptable excipient and one or more of the following: (a) a recombinant antibody of the disclosure; (b) a recombinant nucleic acid of the disclosure; and (c) a recombinant cell of the disclosure.

In another aspect, provided herein are compositions including one or more of the following: (a) one or more barcoded immune-cell marker antibodies and/or barcoded tumor-cell marker antibodies; (b) one or more barcoded therapeutic antibodies; and (c) the barcoded recombinant antibody identified in claim 6 as having specificity for the second tumor sample.

Also provided, in another aspect, are kits that include one or more of the following (a) a recombinant antibody of the disclosure or a functional fragment thereof; (b) a recombinant nucleic acid of the disclosure; and (c) a recombinant cell of the disclosure; and instructions for use thereof.

In another aspect, some embodiments of the disclosure relate to methods for characterizing antibody specificity or target specificity, the methods include: (a) partitioning a single B cell of a plurality of single B cells obtained from a first tumor sample in a partition of a plurality of partitions; (b) determining all or a part of the nucleic acid sequences encoding one or more antibodies produced by the partitioned single B cell; (c) using the determined nucleic acid sequences to produce a recombinant antibody; (d) coupling the recombinant antibody to a reporter oligonucleotide including a reporter barcode sequence to generate a barcoded recombinant antibody; (e) contacting the barcoded recombinant antibody with a second tumor sample, and identifying the recombinant antibody as an antibody having specificity for the tumor sample if the barcoded recombinant antibody is capable of binding to an antigen associated with the second tumor sample; and (f) analyzing RNA expression and protein marker expression for the first and second tumor samples to determine the recombinant antibody specificity and target specificity.

In another aspect, some embodiments of the disclosure relate to methods for enhanced identification of patient-specific or population-specific biomarkers on circulating tumor cells, the methods include: (a) partitioning a single B cell of a plurality of single B cells obtained from a first tumor sample in a partition of a plurality of partitions; (b) determining all or a part of the nucleic acid sequences encoding one or more antibodies produced by the partitioned single B cell; (c) using the determined nucleic acid sequences to produce a recombinant antibody; (d) coupling the recombinant antibody to a reporter oligonucleotide including a reporter barcode sequence to generate a barcoded recombinant antibody; (e) contacting the barcoded recombinant antibody with a second tumor sample, and identifying the recombinant antibody as an antibody having specificity for the tumor sample if the barcoded recombinant antibody is capable of binding to an antigen associated with the second tumor sample; and (f) analyzing RNA expression and protein marker expression for the second tumor sample to identify one or more biomarkers specific for the second tumor sample or for a population of tumor samples.

In another aspect, some embodiments of the disclosure relate to methods for monitoring antigen escape in an individual who has been treated with an antibody-based therapy such as a therapeutic antibody or an antibody-drug conjugate (ADC), the methods include: (a) partitioning a single B cell of a plurality of single B cells obtained from a first tumor sample in a partition of a plurality of partitions; (b) determining all or a part of the nucleic acid sequences encoding one or more antibodies produced by the partitioned single B cell; (c) using the determined nucleic acid sequences to produce a recombinant antibody; (d) coupling the recombinant antibody to a reporter oligonucleotide including a reporter barcode sequence to generate a barcoded recombinant antibody; (e) contacting the barcoded recombinant antibody with a second tumor sample, and identifying the recombinant antibody as an antibody having specificity for the tumor sample if the barcoded recombinant antibody is capable of binding to an antigen associated with the second tumor sample; (f) quantifying binding affinity of a barcoded therapeutic antibody to the second tumor sample, wherein the quantified binding affinity is indicative of the therapeutic antibody's efficacy in treating the tumor; and (g) optionally using the quantified binding affinity to monitor antigen escape from the therapeutic antibody over time.

In another aspect, some embodiments of the disclosure relate to methods for characterizing a potential antigen for an antibody or fragment thereof, the method including: (a) partitioning a single B cell of a plurality of single B cells obtained from a first tumor sample in a partition of a plurality of partitions; (b) determining all or a part of the nucleic acid sequences encoding one or more antibodies produced by the partitioned single B cell; (c) using the determined nucleic acid sequences to produce a recombinant antibody; (d) coupling the recombinant antibody to a reporter oligonucleotide including a reporter barcode sequence to generate a barcoded recombinant antibody; (e) contacting the barcoded recombinant antibody with a second tumor sample, and identifying the recombinant antibody as an antibody having specificity for the tumor sample if the barcoded recombinant antibody is capable of binding to an antigen associated with the second tumor sample; and (f) quantifying binding affinity of the one or more antibodies to the second tumor sample, and using the quantified binding affinity to determine if the one or more antibodies compete with one another for binding to the second tumor sample; and (g) optionally co-associating the quantified binding affinity with RNA expression analysis to identify potential antigen.

In another aspect, provided herein are systems for antibody discovery/management, the systems including: (a) a logic processor; (b) a data compiler communicatively coupled to the logic processor; (c) a stored program code that is executable by the logic processor; and (d) a report engine communicatively coupled to the logic processor, wherein reports produced by the report engine depend upon results from execution of the program code, wherein the program code configures the logic processor to receive from the data compiler information input pertaining to an antibody profile including a preselected set of data input in order to assign a relative performance score to the antibody's tumor specificity based at least in part on the antibody profile, whereby determining the likelihood of the antibody to exhibit one or more tumor specificity attributes as indicated by the assigned relative performance score.

Non-limiting exemplary embodiments of the systems of the disclosure can include one or more of the following features. In some embodiments, the data input includes one or more of the following: (a) antibody sequence data; (b) expression data of biomarkers in the B cell from which the antibody is derived; (c) transcriptomic data for the B cell from which the antibody is derived; (d) whole-exome data; (e) proteomic data; and (f) genomic DNA sequence data from whole-genome sequencing. In some embodiments, the systems of the disclosure further include generating an antibody profile report that contains information relevant to the antibody identified as a tumor-specific antibody. In some embodiments, the antibody profile report is characterized as having an encoding selected from the group consisting of “.doc”; “.pdf”; “.xml”; “.html”; “.jpg”; “.aspx”; “.php”, and a combination of any thereof.

In yet another aspect, provided herein is a non-transitory computer readable medium containing machine executable instructions that when executed cause a processor to perform operations including: receiving an antibody profile including a preselected set of data input; assigning, based at least in part on the antibody profile, a relative performance score to the antibody's tumor specificity; and outputting an antibody profile report for the antibody based upon the assigned performance score. Accordingly, antibody profile reports generated by the compositions and/or systems of the disclosure are also with the scope of this disclosure.

In another aspect of the disclosure, provided herein are methods for generating a recombinant antigen-binding molecule, the method comprising: (a) partitioning a single immune cell of a plurality of single immune cells obtained from a first tumor sample into a partition of a first plurality of partitions; (b) determining all or a part of the nucleic acid sequences encoding one or more antigen-binding molecules produced by the partitioned single immune cell; and (c) using the determined nucleic acid sequences to produce a recombinant antigen-binding molecule. In some embodiments, the single immune cell is a T cell and wherein the one or more antigen-binding molecules produced by the partitioned single immune cell comprises a TCR.

In another aspect of the disclosure, provided herein are methods identifying a tumor-specific antibody, the methods including (a) contacting a barcoded recombinant antibody with a tumor sample, and (b) identifying the barcoded recombinant antibody as a tumor-specific antibody if the barcoded recombinant antibody is capable of binding to an antigen associated with the tumor sample, wherein the barcoded recombinant antibody includes a recombinant antibody coupled to a reporter oligonucleotide including a reporter barcode sequence, wherein the recombinant antibody is identified and/or produced by (i) partitioning a single B cell of a plurality of single B cells obtained from a first tumor sample in a partition of a plurality of partitions, (ii) determining all or a part of the nucleic acid sequences encoding one or more antibodies produced by the partitioned single B cell, and optionally (iii) using the determined nucleic acid sequences to recombinantly produce the recombinant antibody. Non-limiting exemplary embodiments of the methods according to this aspect can include one or more of the following features. In some embodiments, the barcoded recombinant antibody is produced by coupling the recombinant antibody to a reporter oligonucleotide including a reporter barcode sequence to generate a barcoded recombinant antibody.

Each of the aspects and embodiments described herein are capable of being used together, unless excluded either explicitly or clearly from the context of the embodiment or aspect.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 shows an exemplary microfluidic channel structure for partitioning individual biological particles in accordance with some embodiments of the disclosure.

FIG. 2 shows an exemplary microfluidic channel structure for the controlled partitioning of beads into discrete droplets.

FIG. 3 shows an exemplary barcode carrying bead.

FIG. 4 illustrates another example of a barcode carrying bead.

FIG. 5 schematically illustrates an example microwell array.

FIG. 6 schematically illustrates an example workflow for processing nucleic acid molecules.

FIG. 7 schematically illustrates examples of labelling agents.

FIG. 8 depicts an example of a barcode carrying bead.

FIG. 9A-9C schematically depicts an example workflow for processing nucleic acid molecules.

FIG. 10 shows a computer system that is programmed or otherwise configured to implement methods provided herein.

FIG. 11 schematically summarizes the results of experiments performed to identify melanoma V(D)J profiles, illustrating that paired, full-length T and B cell receptor sequences could be identified and obtained from the same sample. In these experiments, paired, full-length T and B cell receptor sequences were generated from all 3 melanoma samples. The T and B cell receptor sequences were generated from the same input material as each other. Left panel: paired clonotype abundance for the top 10 immunoglobulin sequences for each sample are plotted. Right panel: granzyme B expression is shown. GZMB expression was used as a marker for a cytotoxic T cell population in melanoma B, which allows for identification of specific T cell receptor clonotypes associated with specific cell population.

FIG. 12 shows exemplary sequences of melanola B cell receptors to illustrate that updated algorithms can be used to detect framework regions (FWRs), complementarity determining regions CDRs, and indels.

FIG. 13 shows an exemplary microfluidic channel structure for delivering barcode carrying beads to droplets.

FIG. 14 is a schematic diagram showing an example of a barcoded capture probe, as described herein.

FIG. 15 is a schematic illustrating a cleavable capture probe, wherein the cleaved capture probe can enter into a non-permeabilized cell and bind to target analytes within the sample.

FIG. 16 is a schematic diagram of an exemplary multiplexed spatially-barcoded feature.

FIG. 17 is a schematic diagram of an exemplary analyte capture agent.

FIG. 18 is a schematic diagram depicting an exemplary interaction between a feature-immobilized capture probe 524 and an analyte capture agent 526.

### DETAILED DESCRIPTION OF THE DISCLOSURE

The present disclosure relates generally to the development of new or improved immuno-therapeutics, such as recombinant antibodies and pharmaceutical compositions comprising the same for use in treating diseases such as cancer. Some embodiments of the disclosure provide compositions and methods for the analysis and generation of antibodies produced by B cells obtained from tumor samples, using single-cell immune profiling methodologies, so as to produce recombinant antibodies with desired properties. As described in greater detail below, one aspect of the disclosure relates to methods for characterization and/or generation of recombinant monoclonal antibodies from individual B cells derived from tumor samples. In some embodiments, the methods, compositions and systems disclosed herein are used to analyze the sequence of a B cell receptor heavy chain (V_{H}), B cell receptor light chain (V_{L}), or any fragment thereof, e.g., variable regions including VDJ or VJ regions, constant regions, transmembrane regions, fragments thereof, combinations thereof, and combinations of fragments thereof.

An exemplary workflow of the methods disclosed herein generally involves a droplet-based high-throughput analysis of single B cells derived from a tumor sample, and begins with compartmentalizing and/or partitioning of the single B cells into discrete compartments or partitions, followed by determining the sequences of mRNAs encoding V_{H} and/or V_{L} polypeptides produced by the single B cells in the individual droplets, producing the recombinant antibodies based on the determined sequences, and subsequently attaching the recombinant antibodies to an oligonucleotide reporter molecule (e.g., reporter oligonucleotide) comprising a barcoded moiety to generate barcoded recombinant antibodies. These barcoded recombinant antibodies are then tested and/or evaluated for various desired properties using a combination of bioinformatics analysis, gene expression, protein expression, and in vitro and/or in vivo antibody characterization. As illustrated in greater detail below (see, e.g., Example 5), the methods disclosed herein can be used to generate and characterize antibodies produced in tumor samples collected from individuals having cancer, such as, melanoma patients. Remarkably, the methods of the present disclosed can be useful in generating paired, full-length T cell receptor sequences and B cell receptor sequences from tumor samples. Moreover, newly improved algorithms can be used to identify framework regions (FWRs) and complementarity determining regions (CDRs) of the antibodies, as well as amino acid substitutions and indels within these regions.

Various embodiments of the methods described herein involve the compartmentalization, depositing, and/or partitioning of individual B cells derived from a tumor sample into discrete compartments or partitions (referred to interchangeably herein as partitions), where each partition maintains separation of its own contents from the contents of other partitions, which then allows for the analysis, characterization, and/or generation of recombinant monoclonal antibodies from those individual B cells, and optionally allow these antibodies to be attributed back to the B individual cells from which the antibodies are derived. In some embodiments, the recombinant monoclonal antibodies generated by the disclosed methods are coupled to a reporter oligonucleotide comprising a reporter barcode sequence to generate barcoded recombinant antibodies, which can then be used for a multitude of downstream applications, including identification of antibodies having specificity for a tumor, specificity for an individual, or specificity for population of individuals. In some embodiments, the barcoded recombinant antibodies of the disclosure are used in a method of monitoring antigen escape in an individual who has been treated with an antibody-based therapy, such as a therapeutic antibody or an antibody-drug conjugate (ADC). Also provided in some embodiments of the disclosure are recombinant antibodies, compositions and methods useful for the production of such antibodies, as well as kits and systems for antibody discovery and/or management.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “biological particle,” as used herein, generally refers to a discrete biological system derived from a biological sample. The biological particle may be a macromolecule. The biological particle may be a small molecule. The biological particle may be a virus, e.g., a phage. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix. In some embodiments, a biological particle is an analyte carrier, e.g., a cell or constituent of a cell, such as a cell nucleus or organelle.

An “adapter,” an “adaptor,” and a “tag” are terms that are used interchangeably in this disclosure, and refer to moieties that can be coupled to a polynucleotide sequence (in a process referred to as “tagging”) using any one of many different techniques including (but not limited to) ligation, hybridization, and tagmentation. Adapters can also be nucleic acid sequences that add a function, e.g., spacer sequences, primer sequences, primer binding sites, barcode sequences, and unique molecular identifier sequences.

The term “barcode” is used herein to refer to a label, or identifier, that conveys or is capable of conveying information (e.g., information about an analyte in a sample, a bead, and/or a nucleic acid barcode molecule). A barcode can be part of an analyte or nucleic acid barcode molecule, or independent of an analyte or nucleic acid barcode molecule. A barcode can be attached to an analyte or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitates identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

A UMI can include one or more specific polynucleotides sequences, one or more random nucleic acid and/or amino acid sequences, and/or one or more synthetic nucleic acid and/or amino acid sequences. In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample. These nucleotides can be completely contiguous, i.e., in a single stretch of adjacent nucleotides, or they can be separated into two or more separate subsequences that are separated by 1 or more nucleotides.

“Cancer” refers to the presence of cells possessing several characteristics typical of cancer-causing cells, such as uncontrolled proliferation, immortality, metastatic potential, rapid growth and proliferation rate, and certain characteristic morphological features. Some types of cancer cells can aggregate into a mass, such as a tumor, but some cancer cells can exist alone within a subject. A tumor can be a solid tumor, a soft tissue tumor, or a metastatic lesion. As used herein, the term “cancer” also encompasses other types of non-tumor cancers. Non-limiting examples include blood cancers or hematological malignancies, such as leukemia, lymphoma, and myeloma. Cancer can include premalignant, as well as malignant cancers.

The terms “cell”, “cell culture”, “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

As used herein, “isolated” antigen-binding polypeptides, antibodies or antigen-binding fragments thereof, polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or antigen-binding fragments.

As used herein, the term “functional fragment thereof” or “functional variant thereof” relates to a molecule having qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the orthogonal DNA target sequences described herein or the promoter sequence in a nucleic acid construct, or in an engineered response element means that the orthogonal DNA target sequences and the promoters are in-frame and in proper spatial and distance away from a polynucleotide of interest coding for a protein or an RNA to permit the effects of the respective binding by transcription factors or RNA polymerase on transcription.

The term “recombinant” when used with reference to a cell, a nucleic acid, a protein, or a vector, indicates that the cell, nucleic acid, protein or vector has been altered or produced through human intervention such as, for example, has been modified by or is the result of laboratory methods. Thus, for example, recombinant proteins and nucleic acids include proteins and nucleic acids produced by laboratory methods. Recombinant proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; creation of a fusion protein, e.g., a fusion protein comprising an antibody fragment; and addition, deletion, and/or substitution of one or more of nucleic acids in the nucleic acid sequence. The term “recombinant” when used in reference to a cell is not intended to include naturally-occurring cells but encompass cells that have been engineered/modified to include or express a polypeptide or nucleic acid that would not be present in the cell if it was not engineered/modified.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., rat, mouse, cat, dog, cow, pig, sheep, horse, goat, rabbit; and non-mammals, such as amphibians, chicken, reptiles, etc. A subject can be a mammal, preferably a human or humanized animal, e.g., an animal with humanized VDJC loci. The subject may be non-human animals with humanized VDJC loci and knockouts of a target of interest. The subject may be in need of prevention and/or treatment of a disease or disorder such as viral infection or cancer.

A “variant” of a polypeptide, such as an antibody or an immunoglobulin chain (e.g., VH, VL, HC, or LC), refers to a polypeptide comprising an amino acid sequence that has at least about 70-99.9% (e.g., 70%, 72%, 74%, 75%, 76%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%) sequence identity or similarity to a referenced amino acid sequence that is set forth herein. In some embodiments, the term “percent identity,” as used herein in the context of two or more proteins, refers to two or more sequences or subsequences that are the same or have a specified percentage of amino acids that are the same, e.g., about 70%, 72%, 74%, 75%, 76%, 79%, 80%, 81%, 82%, 83%, 84%, 85%, 86%, 87%, 88%, 89%, 90%, 91%, 92%, 93%, 94%, 95%, 96%, 97%, 98%, 9%9, 99.5%, 99.9%, or higher identity over a specified region, when compared and aligned for maximum correspondence over a comparison window or designated region as measured using a BLAST or BLAST 2.0 sequence comparison algorithms with default parameters described below, or by manual alignment and visual inspection. See, e.g., the NCBI web site at ncbi.nlm.nih.gov/BLAST. Similarly, a “variant” of a nucleic acid molecule refers to a nucleic acid molecule comprising a nucleic acid sequence that has at least about 70-99.9% (e.g., 70%, 72%, 74%, 75%, 76%, 79%, 80%, 81%, 82%, 83%, 84%, 85%, 86%, 87%, 88%, 89%, 90%, 91%, 92%, 93%, 94%, 95%, 96%, 97%, 98%, 9%9, 99.5%, 99.9%) sequence identity or similarity to a referenced nucleic acid sequence that is set forth herein. In some embodiments, this definition also refers to, or may be applied to, the complement of a query sequence. In some embodiments, this definition includes sequence comparison performed by a BLAST algorithm wherein the parameters of the algorithm are selected to give the largest match between the respective sequences over the entire length of the respective reference sequences. In some embodiments, this definition also includes sequences that have modifications such as deletions and/or additions (e.g., insertions), as well as those that have substitutions. Such modifications can occur naturally or synthetically. In some embodiments, sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res (1984) 12:387), BLASTP, BLASTN, FASTA (Atschul et al., J Mol Biol (1990) 215:403). In some embodiments, sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. Additional methodologies that can suitably be utilized to determine similarity or identity amino acid sequences include those relying on position-specific structure-scoring matrix (P3SM) that incorporates structure-prediction scores from Rosetta, as well as those based on a length-normalized edit distance as described previously in, e.g., Setcliff et al., Cell Host & Microbe 23(6), May 2018.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Methods of the Disclosure

Some embodiments of the disclosure provide compositions and methods for the analysis and generation of antigen-binding molecules produced by immune cells obtained from tumor samples (e.g., antibodies produced by B cells obtained from tumor samples or TCRs produced by T cells obtained from tumor samples), using single-cell immune profiling methodologies, so as to produce recombinant antigen-binding molecules (e.g., antibodies, TCRs) with desired properties. As described in greater detail below, one aspect of the disclosure relates to methods for characterization and/or generation of recombinant monoclonal antibodies from individual B cells derived from tumor samples. In some embodiments, the methods, compositions and systems disclosed herein are used to determine and analyze the sequence of a B cell receptor heavy chain (V_{H}), B cell receptor light chain (V_{L}), or any fragment thereof, e.g., variable regions including VDJ or VJ regions, constant regions, transmembrane regions, fragments thereof, and combinations of fragments thereof. In some embodiments, the methods further include using the determined nucleic acid sequences to produce a recombinant antibody, and optionally attach the produced recombinant antibody with a reporter molecule, e.g., reporter oligonucleotide to generate a barcoded recombinant antibody, which then can be incorporated into a wide range of downstream applications. For example, in some embodiments, the barcoded recombinant antibodies disclosed herein can be used in methods of identifying tumor-specific antibodies, methods of characterizing antibody specificity or target specificity, methods for enhanced identification of patient-specific or population-specific biomarkers on tumor cells, e.g., circulating tumor cells, methods for monitoring antigen escape in an individual who has been treated with an antibody-based therapy, or methods for characterizing a potential antigen for an antibody or fragment thereof.

### A. Methods for Generating Recombinant Antibodies

In described in more detail below, one aspect of the disclosure relates to new approaches and methods for the analysis, characterization, and/or generation of recombinant antibodies derived from B cells derived from tumor samples, using single-cell immune profiling methodologies, so as to produce recombinant antibodies with desired properties.

In some embodiments, the methods of the disclosure include (a) partitioning at least one single B cell obtained from a tumor sample into individual partitions; (b) determining all or a part of the nucleic acid sequences encoding one or more antibodies produced by the partitioned single B cell; and (c) using the determined nucleic acid sequences to produce a recombinant antibody.

An exemplary workflow for the approaches disclosed herein generally involves compartmentalizing, depositing, and/or partitioning of single B cells from a tumor sample into discrete partitions (e.g., compartments or chambers), where each partition maintains separation of its own contents from the contents of other partitions, which facilitates the analysis, characterization, and/or generation of recombinant monoclonal antibodies derived from those single B cells. For example, in some embodiments, single B cells derived from a tumor sample are individually partitioned to discrete droplets together with hydrogel beads coupled with nucleic acid barcode molecules to generate droplets that contain a single B cell and a single bead. In some embodiments, single B cells are individually co-partitioned along with a solid substrate (e.g., a gel bead) coupled to a nucleic acid barcode molecule, and other reagents such as reverse transcriptase, reducing agent and dNTPs into a partition (e.g., a droplet in an emulsion). In some embodiments, single B cells are individually partitioned into microwells. Exemplary methods and systems for microwell partitioning are described herein.

### B. Methods for Generating Recombinant TCRs

As described in more detail below, one aspect of the disclosure relates to new approaches and methods for the analysis, characterization, and/or generation of recombinant TCRs derived from T cells derived from tumor samples, using single-cell immune profiling methodologies, so as to produce recombinant TCRs with desired properties.

In some embodiments, the methods of the disclosure include (a) partitioning at least one single T cell obtained from a tumor sample into individual partitions; (b) determining all or a part of the nucleic acid sequences encoding one or more TCRs produced by the partitioned single T cell; and (c) using the determined nucleic acid sequences to produce a recombinant TCR.

An exemplary workflow for the approaches disclosed herein generally involves compartmentalizing, depositing, and/or partitioning of single T cells from a tumor sample into discrete partitions (e.g., compartments or chambers), where each partition maintains separation of its own contents from the contents of other partitions, which facilitates the analysis, characterization, and/or generation of recombinant monoclonal TCRs derived from those single T cells. For example, in some embodiments, single T cells derived from a tumor sample are individually partitioned to discrete droplets together with hydrogel beads coupled with nucleic acid barcode molecules to generate droplets that contain a single T cell and a single bead. In some embodiments, single T cells are individually co-partitioned along with a solid substrate (e.g., a gel bead) coupled to a nucleic acid barcode molecule, and other reagents such as reverse transcriptase, reducing agent and dNTPs into a partition (e.g., a droplet in an emulsion). In some embodiments, single T cells are individually partitioned into microwells. Exemplary methods and systems for microwell partitioning are described herein.

For any of the workflows described above, after cell lysis and reverse transcription of V_{H} and V_{L} mRNAs produced in the droplets, the complementary DNAs from each cell carry a unique barcode that allows cognate V_{H} and V_{L} pairs to be identified by nucleic acid sequencing, followed by gene synthesis, cloning, and production of selected recombinant antibodies or TCRs.

As discussed above, a skilled artisan in the art will understand that the term partition generally refers to a discrete space or volume that may be suitable to contain one or more cells, one or more species of features or compounds, or to conduct one or more reactions. In some embodiments, the partitions are physical compartments such as, droplets, flowcells, reactions chambers, or wells (e.g., microwells). In some embodiments, the partitions of the disclosures are droplets. In some embodiments, the droplets can be a first phase (e.g., aqueous phase) in a second phase (e.g., oil) immiscible with the first phase. In some embodiments, the droplets can be a first phase in a second phase that does not phase separate from the first phase, such as, for example, a capsule (e.g., microcapsule) or liposome in an aqueous phase. In some embodiments, a partition of the disclosure can optionally include one or more other partitions, e.g., inner partitions or sub-partitions. Additional information regarding methods and systems for partitioning individual cells or cell populations in a plurality of partitions can be found in, for example, PCT Publication No. WO2018075693A1, which is hereby incorporated by reference.

In some embodiments, the methods further include partitioning cell lysis reagents into the partition in order to release the contents of the partitioned single cell (e.g., single B cell or single T cell. In some embodiments, the methods further include using the one or more nucleic acid barcode molecules and one or more nucleic acid analytes of the partitioned single cell (e.g., B cell or T cell) to generate one or more barcoded nucleic acid molecule comprising a coding sequence for an antigen-binding molecule (e.g., antibody) produced by the partitioned single cell, or any fragments thereof such as a B cell receptor heavy chain (V H), B cell receptor light chain (V_{L}), or any fragment thereof, or T cell receptor alpha chain and beta chain, or any fragment thereof, e.g., variable regions including VDJ or VJ regions, constant regions, transmembrane regions, fragments thereof, and combinations of fragments thereof.

Nucleic acid sequencing of the barcoded nucleic acid molecule can be used to determine nucleic acid sequences that encode one or more antigen-binding molecules produced by the partitioned single cells (e.g., single B cells or single T cells). For example, once the single B cells are partitioned in individual droplets, nucleic acid sequencing can be used to determine nucleic acid sequences that encode one or more antibodies produced by the partitioned single B cells.

A plethora of different approaches, systems, and techniques for nucleic acid sequencing, including next-generation sequencing (NGS) methods, can be used to determine the nucleic acid sequences encoding the antibodies produced by the partitioned single B cells. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification.

Non-limiting examples of nucleic acid sequencing methods include Maxam-Gilbert sequencing and chain-termination methods, de novo sequencing methods including shotgun sequencing and bridge PCR, next-generation methods including Polony sequencing, 454 pyrosequencing, Illumina sequencing, SOLiD™ sequencing, Ion Torrent semiconductor sequencing, HeliScope single molecule sequencing, and SMRT® sequencing.

Other examples of methods for sequencing genetic material include, but are not limited to, DNA hybridization methods, restriction enzyme digestion methods, Sanger sequencing methods, ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, Solexa Genome Analyzer sequencing, MS-PET sequencing, and any combinations thereof.

Sequence analysis of the nucleic acid molecules, including barcoded nucleic acid molecules (e.g., barcoded cDNA), can be direct or indirect. Thus, the sequence analysis substrate (which can be viewed as the molecule which is subjected to the sequence analysis step or process) can be the barcoded nucleic acid molecule or it can be a molecule which is derived therefrom (e.g., a complement thereof). Thus, for example, in the sequence analysis step of a sequencing reaction, the sequencing template can be the barcoded nucleic acid molecule or it can be a molecule derived therefrom. For example, a first and/or second strand DNA molecule can be directly subjected to sequence analysis (e.g., sequencing), i.e., can directly take part in the sequence analysis reaction or process (e.g., the sequencing reaction or sequencing process, or be the molecule which is sequenced or otherwise identified). Alternatively, the barcoded nucleic acid molecule can be subjected to a step of second strand synthesis or amplification before sequence analysis (e.g., sequencing or identification by another technique). The sequence analysis substrate (e.g., template) can thus be an amplicon or a second strand of a barcoded nucleic acid molecule.

In some embodiments, both strands of a double stranded molecule (e.g., cDNA) can be subjected to sequence analysis. In some embodiments, single stranded molecules (e.g., barcoded nucleic acid molecules) can be sequenced.

In some embodiments, all or a part of the nucleic acid sequences encoding one or more antigen-binding molecules produced by the partitioned single immune cell (e.g., encoding one or more antibodies produced by the partitioned single B cell) can be determined by using a whole-exome sequencing technique (WES), which generally involves sequencing all of the protein-coding regions of genes in a cellular genome (often referred to as the exome). A general workflow of whole-exome sequencing includes two steps: the first step involves selecting only the subset of DNA that encodes proteins. These regions are known as exons (for example, humans have about 180,000 exons, constituting about 1% of the human genome). The second step involves sequencing the exonic DNA using any suitable high-throughput DNA sequencing technology.

Production of Recombinant Antigen-Binding Molecules (e.g., Recombinant Antibodies or Recombinant TCRs)

In some embodiments, the method further includes generating a recombinant antigen-binding molecule using the determined nucleic acid sequences of the partitioned immune cell. In some embodiments, the method includes further generating a recombinant antibody using the determined nucleic acid sequences of the partitioned B cell. In some embodiments, the method includes further generating a recombinant TCR using the determined nucleic acid sequences of the partitioned T cell. One skilled in the art will appreciate that the determined nucleic acid sequences of the V_{H} and V_{L} mRNAs can be used to construct a full-length gene encoding a desired recombinant antibody. For example, a DNA oligomer containing a full-length nucleotide sequence coding for a given V_{H} and V_{L} domain of the desired antibody can be synthesized. In addition or alternatively, several small oligonucleotides coding for portions of the desired recombinant antibody can be synthesized and then ligated. The individual oligonucleotides generally contain 5′ or 3′ overhangs for complementary assembly.

In addition to generating desired antibodies or TCRs via expression of nucleic acid molecules that have been altered by recombinant molecular biological techniques, a subject recombinant antibody or TCR in accordance with the present disclosure can be chemically synthesized. Chemically synthesized polypeptides are routinely generated by those of skill in the art.

Once assembled (by synthesis, recombinant methodologies, site-directed mutagenesis or other suitable techniques), the DNA sequences encoding a recombinant antigen-binding molecule (e.g., antibody or TCR) as disclosed herein can be inserted into an expression vector and operably linked to an expression control sequence appropriate for expression of the recombinant antibody in the desired transformed host. Proper assembly can be confirmed by nucleotide sequencing, restriction mapping, and expression of a biologically active polypeptide in a suitable host. As is known in the art, in order to obtain high expression levels of a transfected gene in a host, care should be taken to ensure that the gene encoding the recombinant antibody is operably linked to transcriptional and translational expression control sequences that are functional in the chosen expression host.

In some embodiments, a method of the disclosure further involves including coupling a reporter oligonucleotide including a reporter barcode sequence to the recombinant antibody to generate a barcoded recombinant antibody. In some embodiments, the reporter barcode sequence of the reporter oligonucleotide includes a unique identifier for the recombinant antibody. In some embodiments, the unique identifier for the recombinant is a nucleic acid identifier. In some embodiments, the method of the disclosure further including determining all or a part of the nucleic acid identifier to identify the barcoded recombinant antibody. In some embodiments, the reporter oligonucleotide comprise an adapter region that allows for downstream analysis of the recombinant antibody. In some embodiments, the adapter region comprises a primer binding site and/or a cleavage site.

In some embodiments, the single B cell is obtained from a first tumor sample. In some embodiments, the method of the disclosure further including contacting the barcoded recombinant antibody to a second tumor sample or a single tumor cell obtained from the second tumor sample. In some embodiments, the method further including identifying the recombinant antibody as an antibody having specificity for the second tumor sample if the barcoded recombinant antibody is capable of binding to an antigen associated with the second tumor sample.

In some embodiments, a method of the disclosure further involves including coupling a reporter oligonucleotide including a reporter barcode sequence to the recombinant TCR to generate a barcoded recombinant TCR. In some embodiments, the reporter barcode sequence of the reporter oligonucleotide includes a unique identifier for the recombinant TCR. In some embodiments, the unique identifier for the recombinant is a nucleic acid identifier. In some embodiments, the method of the disclosure further including determining all or a part of the nucleic acid identifier to identify the barcoded recombinant TCR. In some embodiments, the reporter oligonucleotide comprise an adapter region that allows for downstream analysis of the recombinant TCR. In some embodiments, the adapter region comprises a primer binding site and/or a cleavage site.

In some embodiments, the single T cell is obtained from a first tumor sample. In some embodiments, the method of the disclosure further including contacting the barcoded recombinant TCR to a second tumor sample or a single tumor cell obtained from the second tumor sample. In some embodiments, the method further including identifying the recombinant TCR as an TCR having specificity for the second tumor sample if the barcoded recombinant TCR is capable of binding to an antigen associated with the second tumor sample.

Attachment (coupling) of the reporter oligonucleotides to a recombinant antibody or recombinant TCR can be performed via any of the methods described herein for attachment (coupling) of reporter oligonucleotides to labelling agents (such as a protein, e.g., an antibody or antibody fragment). For example, attachment (coupling) or the reporter oligonucleotides to a recombinant antibody or recombinant TCR can be performed using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or an streptavidin linker in monomeric or multimeric form (e.g., tetramic form of streptavidin). Those of skill in the art will recognize that a streptavidin monomer encompasses streptavidin molecules with 1 biotin binding site, while a streptavidin multimer encompasses strepatavidin molecules with more than 1 biotin binding site. For example, a streptavidin tetramer has 4 biotin binding sites. However, a skilled artisan will also recognize that in a streptavidin tetramer does not necessarily comprise 4 streptavidins complexed together. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, can be used to couple reporter oligonucleotides to labelling agents. Commercially available kits, such as those from Thunderlink and Abcam, and techniques common in the art can be used to couple reporter oligonucleotides to labelling agents as appropriate. In another example, a labelling agent is indirectly (e.g., via hybridization) coupled to a reporter oligonucleotide including a barcode sequence that identifies the label agent. For instance, the labelling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

### Tumor Samples

The first and/or second tumor sample can be any biological sample comprising tumor cells. For example, the first and/or second tumor sample can be a tissue sample, such as a biopsy, core biopsy, needle aspirate, or fine needle aspirate. The first and/or second tumor sample can be a fluid sample, such as a blood sample, urine sample, or saliva sample. The first and/or second tumor sample can be a skin sample. The first and/or second tumor sample can be a cheek swab. The first and/or second tumor sample can be a plasma or serum sample. In some embodiments, the first and/or the second tumor sample is independently derived from a solid tumor, a soft tissue tumor, a non-solid tumor, a metastatic lesion, a circulating tumor cell (CTC) population. The first and/or second tumor sample can comprise an intact tissue sample. The first and/or second tumor sample can comprise a dissociated cell sample. In some embodiments, the first and/or the second tumor sample can be a tumor cell line or a patient derived xenograft (PDX). In some embodiments, the first and the second tumor samples are derived from the same subject. In some embodiments, the first and the second tumor samples are derived from the different subjects. In some embodiments, the first and the second tumor samples are derived from the same tumor type. In some embodiments, the first and the second tumor samples are derived from the different tumor types. For example, in some embodiments, the single immune cells (e.g., B cells, T cells) are obtained from a first tumor sample which is a solid tumor and the second tumor is a non-solid tumor sample. In another example, the first sample and the second samples are different samples derived from the same tumor tissue. For example, in some embodiments, the first tumor sample is obtained from a solid tumor and the second tumor sample is a CTC population. In some embodiments, the first and the second tumor samples are derived from the same type of tumor, but are collected at different times and/or under different conditions.

In some embodiments, the single B cell derived from the first tumor sample is a memory B cell, a naïve mature B cell, a primary B cell, or a tumor-infiltrating B cell (TIL-B). In some embodiments, the single B cell is a tumor-infiltrating B cell. In some embodiments, the method of the disclosure further includes, prior to the partitioning step, dissociating the first tumor sample to generate a population of single cells, e.g., a dissociated cell sample. In some embodiments, the method further includes isolating and/or enriching the plurality of single B cells prior to the partitioning step. Methods for isolating B cells e.g., B cells from a tumor sample) and/or enriching B cell populations are known in the art.

In some embodiments, when a tumor sample is not a CTC population, the method further includes dissociating the tumor sample to generate a population of single tumor cells. For example, in some embodiment, the second tumor sample is a solid tumor sample and the method further includes dissociating the second tumor sample to generate a population of single tumor cells prior to contacting the barcoded recombinant antibody to single tumor cells of the generated single tumor cell population.

In some embodiments, the methods further includes isolating and/or enriching the single tumor cells prior to contacting the barcoded recombinant antibody to a single tumor cells. In some embodiments, the single tumor cell is a circulating tumor cell (CTC), where the isolation and/or enrichment of CTC can be achieved by using one or more CTC capture methods and systems known in the art. Non-limiting examples of CTC capture methods suitable for the present disclosure include those commercially available and involve the use of a support comprising a flowpath (e.g., channel), such as Biofludica platform or Biocep platform. In some embodiments where Biofludica CTC capture systems are used, the flowpath can be generally coupled with antibodies configured to bind the CTC. For example, in some embodiments, a BioFluidica's CTC system can be used with a highly efficient CTC capture bed comprising 50 to 500 sinusoidally-shaped channels. These channels are coated with antibodies which are chemically immobilized onto the surfaces of the capture bed. These antibodies are configured to bind and isolate specific CTCs from tumor samples. In some embodiments where Biocept CTC capture methods are used, the flowpath comprises a microchannel that is further configured to include obstacles of different sizes, e.g., irregular arrangement of multiple sized posts that are designed to disrupt internal stream-lines for superior CTC capture. Additionally, in some embodiments, the interior of the Biocept microchannel can be chemically derivatized to enable the capture of CTCs tagged with specific antibodies.

### C. Methods for Characterizing Tumor Response to One or More Antibody Therapeutics

In an aspect, provided herein are methods for characterizing response of a tumor to one or more antibody therapeutics. In some embodiments, the method of the disclosure includes contacting a tumor sample (e.g., a first or second tumor sample described herein) with a composition including one or more barcoded antibodies and/or functional fragments thereof (e.g., a barcoded antibody cocktail). In some embodiments, the tumor sample is a dissociated cell sample. In some embodiments, the tumor sample is an intact tissue sample. In some embodiments, the method comprises contacting a single tumor cell (e.g., a partitioned single tumor cell) from the tumor sample with the composition including one or more barcoded antibodies and/or functional fragments thereof (e.g., a barcoded antibody cocktail). Non-limiting examples of barcoded antibodies suitable for the compositions and methods described herein include barcoded antibodies and functional fragments thereof having specificity for immune cell markers, tumor-cell markers, and/or specificity for a tumor sample. Accordingly, in some embodiments, the method of the disclosure includes contacting a tumor sample or single tumor cell obtained from the tumor sample with a composition including one or more of the following: (i) one or more barcoded immune-cell marker antibodies and/or functional fragments thereof; (ii) one or more barcoded tumor-cell marker antibodies and functional fragments thereof; (iii) one or more barcoded therapeutic antibodies and functional fragments thereof; and (iv) one or more barcoded recombinant antibodies identified in the present disclosure as having specificity for a tumor sample. In some embodiments, the tumor sample or single tumor cell obtained from the tumor sample is contacted with a composition including two or more of (i)-(iv), three or more of (i)-(iv), or all of (i)-(iv).

In some embodiments, the method comprises generating the barcoded recombinant antibodies of (iv) using determined nucleic acid sequences of single B cells obtained from a first tumor sample; and contacting a second tumor sample with the composition including one or more of (i)-(iv).

In some embodiments, the barcoded antibodies are each coupled to a reporter oligonucleotide including a reporter barcode sequence. In some embodiments, to facilitate downstream analyses, the reporter barcode sequence coupled to a barcoded antibody is distinguishable from reporter barcode sequences coupled to other barcoded antibodies.

In some embodiments, one or more of the antibodies are monoclonal antibodies. In some embodiments, one or more of the antibodies are polyclonal antibodies. In some embodiments, one or more of the antibodies are multi-specific antibodies (e.g., bispecific antibodies). Functional fragments of the antibodies suitable for the methods described herein can include F(ab) fragments, Fab′ fragments, F(ab′)2 fragments, FIT domains, and Fc domains.

### Therapeutic Antibodies

Therapeutic antibodies that can be used are antibodies that have been approved for human administration for the treatment of a disease, such as cancer or antibodies that are being tested for preclinical and/or clinical trials. In some embodiments, a therapeutic antibody is an antibody of known sequence that is contemplated for use in treating a physiological condition or disease, such as cancer, in a human.

In some embodiments, the method of the disclosure includes contacting the single tumor cell obtained from the second tumor sample with a composition including one or more therapeutic antibodies that are drug candidates or FDA approved drugs or therapeutics, such as monoclonal antibodies that are approved by the FDA for therapeutic use. The one or more therapeutic antibodies may be barcoded. Non-limiting examples of FDA approved monoclonal antibodies are provided in Table 1 below.

TABLE 1
Exemplary FDA-approved therapeutic monoclonal antibodies.

| Antibody | Brand name | Type | Target |
|---|---|---|---|
| abciximab | ReoPro | chimeric Fab | GPIIb/IIIa |
| adalimumab | Humira | fully human | TNF |
| adalimumab-atto | Amjevita | fully | TNF |
| | | human, biosimilar | |
| ado-trastuzumab emtansine | Kadcyla | humanized, antibody- | HER2 |
| | | drug conjugate | |
| alemtuzumab | Campath, | humanized | CD52 |
| | Lemtrada | | |
| alirocumab | Praluent | fully human | PCSK9 |
| atezolizumab | Tecentriq | humanized | PD-L1 |
| atezolizumab | Tecentriq | humanized | PD-L1 |
| avelumab | Bavencio | fully human | PD-L1 |
| basiliximab | Simulect | chimeric | IL2RA |
| belimumab | Benlysta | fully human | BLyS |
| bevacizumab | Avastin | humanized | VEGF |
| bezlotoxumab | Zinplava | fully human | Clostridium difficile |
| | | | toxin B |
| blinatumomab | Blincyto | mouse, bispecific | CD19 |
| brentuximab vedotin | Adcetris | chimeric, antibody- | CD30 |
| | | drug conjugate | |
| brodalumab | Siliq | chimeric | IL17RA |
| canakinumab | Ilaris | fully human | IL1B |
| capromab pendetide | ProstaScint | murine, radiolabeled | PSMA |
| certolizumab pegol | Cimzia | humanized | TNF |
| cetuximab | Erbitux | chimeric | EGFR |
| daclizumab | Zenapax | humanized | IL2RA |
| daclizumab | Zinbryta | humanized | IL2R |
| daratumumab | Darzalex | fully human | CD38 |
| denosumab | Prolia, Xgeva | fully human | RANKL |
| dinutuximab | Unituxin | chimeric | GD2 |
| dupilumab | Dupixent | fully human | IL4RA |
| durvalumab | Imfinzi | fully human | PD-L1 |
| eculizumab | Soliris | humanized | Complement |
| | | | component 5 |
| elotuzumab | Empliciti | humanized | SLAMF7 |
| evolocumab | Repatha | fully human | PCSK9 |
| golimumab | Simponi | fully human | TNF |
| golimumab | Simponi Aria | fully human | TNF |
| ibritumomab tiuxetan | Zevalin | murine, | CD20 |
| | | radioimmunotherapy | |
| idarucizumab | Praxbind | humanized Fab | dabigatran |
| infliximab | Remicade | chimeric | TNF alpha |
| infliximab-abda | Renflexis | chimeric, biosimilar | TNF |
| infliximab-dyyb | Inflectra | chimeric, biosimilar | TNF |
| ipilimumab | Yervoy | fully human | CTLA-4 |
| ixekizumab | Taltz | humanized | IL17A |
| mepolizumab | Nucala | humanized | IL5 |
| natalizumab | Tysabri | humanized | alpha-4 integrin |
| necitumumab | Portrazza | fully human | EGFR |
| nivolumab | Opdivo | fully human | PD-1 |
| nivolumab | Opdivo | fully human | PD-1 |
| obiltoxaximab | Anthem | chimeric | Protective antigen of |
| | | | the Anthrax toxin |
| obinutuzumab | Gazyva | humanized | CD20 |
| ocrelizumab | Ocrevus | humanized | CD20 |
| ofatumumab | Arzerra | fully human | CD20 |
| olaratumab | Lartruvo | fully human | PDGFRA |
| omalizumab | Xolair | humanized | IgE |
| palivizumab | Synagis | humanized | F protein of RSV |
| panitumumab | Vectibix | fully human | EGFR |
| pembrolizumab | Keytruda | humanized | PD-1 |
| pertuzumab | Perjeta | humanized | HER2 |
| ramucirumab | Cyramza | fully human | VEGFR2 |
| ranibizumab | Lucentis | humanized | VEGFR1, VEGFR2 |
| raxibacumab | Raxibacumab | fully human | Protective antigen |
| | | | of Bacillus anthracis |
| reslizumab | Cinqair | humanized | IL5 |
| rituximab | Rituxan | chimeric | CD20 |
| secukinumab | Cosentyx | fully human | IL17A |
| siltuximab | Sylvant | chimeric | IL6 |
| tocilizumab | Actemra | humanized | IL6R |
| tocilizumab | Actemra | humanized | IL6R |
| trastuzumab | Herceptin | humanized | HER2 |
| ustekinumab | Stelara | fully human | IL12 |
| ustekinumab | Stelara | fully human | IL12, IL23 |
| vedolizumab | Entyvio | humanized | integrin receptor |
| sarilumab | Kevzara | fully human | IL6R |
| rituximab and hyaluronidase | Rituxan | chimeric, co- | CD20 |
| | Hycela | formulated | |
| guselkumab | Tremfya | fully human | IL23 |
| inotuzumab ozogamicin | Besponsa | humanized, antibody- | CD22 |
| | | drug conjugate | |
| adalimumab-adbm | Cyltezo | fully | TNF |
| | | human, biosimilar | |
| gemtuzumab ozogamicin | Mylotarg | humanized, antibody- | CD33 |
| | | drug conjugate | |
| bevacizumab-awwb | Mvasi | humanized, biosimilar | VEGF |
| benralizumab | Fasenra | humanized | interleukin-5 receptor |
| | | | alpha subunit |
| emicizumab-kxwh | Hemlibra | humanized, bispecific | Factor IXa, Factor X |
| trastuzumab-dkst | Ogivri | humanized, biosimilar | HER2 |
| infliximab-qbtx | Ixifi | chimeric, biosimilar | TNF |
| ibalizumab-uiyk | Trogarzo | humanized | CD4 |
| tildrakizumab-asmn | Ilumya | humanized | IL23 |
| burosumab-twza | Crysvita | fully human | FGF23 |
| erenumab-aooe | Aimovig | fully human | CGRP receptor |

In some embodiments, the method of the disclosure includes contacting the single tumor cell obtained from the second tumor sample with a composition including one or more therapeutic antibodies which can be, for example, abagovomab, abatacept, abciximab, abituzumab, abrilumab, actoxumab, adalimumab, adecatumab, aducanumab, aflibercept, afutuzymab, alacizumab, alefacept, alemtuzumab, alirocumab, altumomab, amatixumab, anatumomab, anetumab, anifromumab, anrukinzumab, apolizumab, arcitumomab, ascrinvacumab, aselizumab, atezolizumab, atinumab, altizumab, atorolimumab, bapineuzumab, basiliximab, bavituximab, bectumomab, begelomab, belatacept, belimumab, benralizumab, bertilimumab, besilesomab, bevacizumab, bezlotoxumab, biciromab, bimagrumab, bimekizumab, bivatuzumab, blinatumomab, blosozumab, bococizumab, brentuximab, briakimumab, brodalumab, brolucizumab, bronticizumab, canakinumab, cantuzumab, caplacizumab, capromab, carlumab, catumaxomab, cedelizumab, certolizumab, cetixumab, citatuzumab, cixutumumab, clazakizumab, clenoliximab, clivatuzumab, codrituzumab, coltuximab, conatumumab, concizumab, crenezumab, dacetuzumab, daclizumab, dalotuzumab, dapirolizumab, daratumumab, dectrekumab, demcizumab, denintuzumab, denosumab, derlotixumab, detumomab, dinutuximab, diridavumab, dorlinomab, drozitumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab, enlimomab, enoblituzumab, enokizumab, enoticumab, ensituximab, epitumomab, epratuzomab, erlizumab, ertumaxomab, etanercept, etaracizumab, etrolizumab, evinacumab, evolocumab, and exbivirumab.

Additional therapeutic antibodies suitable for the compositions, systems, and methods described herein include, but are not limited to, fanolesomab, faralimomab, farletuzomab, fasimumab, felvizumab, fezkimumab, ficlatuzumab, figitumumab, firivumab, flanvotumab, fletikumab, fontolizumab, foralumab, foravirumab, fresolimumab, fulramumab, futuximab, galiximab, ganitumab, gantenerumab, gavilimomab, gemtuzumab, gevokizumab, girentuximab, glembatumumab, golimumab, gomiliximab, guselkumab, ibalizumab, Iibritumomab, icrucumab, idarucizumab, igovomab, imalumab, imciromab, imgatuzumab, inclacumab, indatuximab, indusatumab, infliximab, intetumumab, inolimomab, inotuzumab, ipilimumab, iratumumab, isatuximab, itolizumab, ixekizumab, keliximab, labetuzumab, lambrolizumab, lampalizumab, lebrikizumab, lemalesomab, lenzilumab, lerdelimumab, lexatumumab, libivirumab, lifastuzumab, ligelizumab, lilotomab, lintuzumab, lirilumab, lodelcizumab, lokivetmab, lorvotuzumab, lucatumumab, lulizumab, lumiliximab, lumretuzumab, mapatumumab, margetuximab, maslimomab, mavrilimumab, matuzumab, mepolizumab, metelimumab, milatuzumab, minetumomab, mirvetuximab, mitumomab, mogamulizumab, morolimumab, motavizumab, moxetumomab, muromonab-CD3, nacolomab, namilumab, naptumomab, narnatumab, natalizumab, nebacumab, necitumumab, nemolizumab, nerelimomab, nesvacumab, nimotuzumab, nivolumab, nofetumomab, obiltoxaximab, obinutuzumab, ocaratuzumab, ocrelizumab, odulimomab, ofatumumab, olaratumab, olokizumab, omalizumab, onartuzumab, ontuxizumab, opicinumab, oportuzumab, oregovomab, orticumab, otelixizumab, oltertuzumab, oxelumab, ozanezumab, ozoralizumab, pagibaximab, palivizumab, panitumumab, pankomab, panobacumab, parsatuzumab, pascolizumab, pasotuxizumab, pateclizumab, patritumab, pembrolizumab, pemtumomab, perakizumab, pertuzumab, pexelizumab, pidilizumab, pinatuzumab, pintumomab, polatuzumab, ponezumab, priliximab, and pritumumab.

Further non-limiting examples of suitable therapeutic antibodies include quilizumab, racotumomab, radretumab, rafivirumab, ralpancizumab, ramucirumab, ranibizumab, raxibacumab, refanezumab, regavirumab, reslizumab, rilonacept, rilotumumab, rinucumab, rituximab, robatumumab, roledumab, romosozumab, rontalizumab, rovelizumab, ruplizumab, sacituzumab, samalizumab, sarilumab, satumomab, secukimumab, seribantumab, setoxaximab, sevirumab, sibrotuzumab, sifalimumab, siltuximab, siplizumab, sirukumab, sofituzumab, solanezumab, solitomab, sonepcizumab, sontuzumab, stamulumab, sulesomab, suvizumab, tabalumab, tacatuzumab, tadocizumab, talizumab, tanezumab, taplitumomab, tarextumab, tefibazumab, telimomab aritox, tenatumomab, teneliximab, teplizumab, tesidolumab, TGN 1412, ticlimumab, tildrakizumab, tigatuzumab, TNX-650, tocilizumab, toralizumab, tosatoxumab, tositumomab, tovetumab, tralokimumab, trastuzumab, TRBS07, tregalizumab, tremelimumab, trevogrumab, tucotuzumab, tuvirumab, ublituximab, ulocuplumab, urelumab, urtoxazumab, ustekimumab, vandortuzumab, vantictumab, vanucizumab, vapaliximab, varlimumab, vatelizumab, vedolizumab, veltuzumab, vepalimomab, vesencumab, visilizumab, volocixumab, vorsetuzumab, votumumab, zalutumimab, zanolimumab, zatuximab, ziralimumab, ziv-aflibercept, and zolimomab.

In some embodiments, the therapeutic antibodies selected from the group consisting of abciximab, abciximab, adalimumab, aducanumab, alacizumab, alemtuzumab, alirocumab, alirocumab, ascrinvacumab, atezolizumab, atinumab, bapineuzumab, basiliximab, basiliximab, belimumab, bevacizumab, blinatumomab, blosozumab, bococizumab, brentuximab, canakinumab, caplacizumab, capromab, certolizumab, cetuximab, crenezumab, daclizumab, daratumumab, demcizumab, denosumab, denosumab, dinutuximab, ecukinumab, eculizumab, eculizumab, efalizumab, elotuzumab, enoticumab, etaracizumab, evinacumab, evolocumab, evolocumab, fasinumab, fulranumab, gantenerumab, golimumab, ibritumomab, icrucumab, idarucizumab, idarucizumab, inciacumab, infliximab, ipilimumab, mepolizumab, natalizumab, necitumumab, nesvacumab, nivolumab, obinutuzumab, ofatumumab, omalizumab, opicinumab, orticumab, ozanezumab, palivizumab, palivizumab, panitumumab, pembrolizumab, pertuzumab, ponezumab, ralpancizumab, ramucirumab, ramucirumab, ranibizumab, raxibacumab, refanezumab, rinucumab, rituximab, romosozumab, siltuximab, solanezumab, stamulumab, tadocizumab, tanezumab, tocilizumab, trastuzumab, ustekinumab, vedolizumab, and vesencumab.

### Immune-Cell Marker Antibodies

In some embodiments, the methods of the disclosure include contacting the single tumor cell obtained from the second tumor sample with a composition including one or more barcoded immune-cell marker antibodies, e.g., antibodies having specificity for one or more immune cells. For example, the specificity of the recombinant antibodies as described herein for an immune cell can be for a marker expressed on the surface of the immune cell. Examples of immune-cell marker antibodies include, but are not limited to, antibodies having specificity for one or more molecular markers of B cells, T cells, monocytes, macrophages, granulocytes (e.g., basophil, eosinophil, and neutrophil), dendritic cells, natural killer (NK) cells, and/or natural killer T (NKT) cells. For example, exemplary extracellular markers for B cells can include, but are not limited to, CD2, CD5, CD19, CD20, CD21/CD35 (CR2/CR1), CD22, CD23, CD40, CD45R/B220, CD69, CD70, CD74, CD79a (Igα), CD79b (Igβ), CD80, CD86, CD93 (C1Rqp), CD137 (4-1BB), CD138 (Syndecan-1), CD252 (OX40L), CD267, CD268 (BAFF-R), CD279 (PD1), HLA-DR, IgG, IgD, and IgM. For T cells, exemplary extracellular markers suitable for the compositions and methods of the disclosure can include, but are not limited to, CD3, CD4, CD8, CD25, CD39, CD43, CD45RO, CD62L, CD73, CD103, CD134, CD152 (CTLA-4), CD194 (CCR4), and CD223. For monocytes, exemplary extracellular markers can include, but are not limited to, CD14 and CD16.

In some embodiments, one or more immune-cell marker antibodies have specificity for a molecular marker of macrophages. Exemplary extracellular markers suitable for the compositions, systems, and methods described herein can include, but are not limited to, CD11a, CD11b, CD11c, CD14, CD15 (SSEA-1), CD16/32, CD33, CD64, CD68, CD80, CD85k (ILT3), CD86, CD105 (Endoglin), CD107b, CD115, CD163, CD195 (CCR5), CD282 (TLR2), and CD284 (TLR4). For basophils, exemplary extracellular markers can include, but are not limited to, CD13, CD44, CD54, CD63, CD69, CD107a, CD123, CD193 (CCR3), CD203c, FcεRIα, IgE, and TLR4.

In some embodiments, one or more immune-cell marker antibodies have specificity for a molecular marker of granulocytes, e.g., eosinophil. Exemplary extracellular markers for eosinophil that are suitable for the compositions and methods of the disclosure can include, but are not limited to, C3AR, CD15 (SSEA-1), CD23, CD49d, CD52, CD53, CD88, CD129, CD183, CD191, CD193, CD244 (2B4), CD294, and CD305. For neutrophils, exemplary extracellular markers can include, but are not limited to, CD10, CD11b, CD11c, CD13, CD14, CD15 (SSEA-1), CD16/32, CD31, CD33, CD62L, CD64, CD66b, CD88, and CD114 (G-CSFR). For myeloid dendritic cells, exemplary extracellular markers can include, but are not limited to, CD1a, CD1b, CD1c, CD4, CD11b, CD11c, CD40, CD49d, CD80, CD83, CD86, CD197 (CCR7), CD205 (DEC-205), CD207 (Langerin), CD209 (DC-SIGN), CD273 (B7-DC, PD-L2), and CD304 (Neuropilin-1).

In some embodiments, one or more immune-cell marker antibodies have specificity for a molecular marker of NK cells. Exemplary extracellular markers for NK cells suitable for the compositions and methods described herein can include, but are not limited to, CD11b, CD11c, CD16/32, CD49b, CD56 (NCAM), CD57, CD69, CD94, CD122, CD158 (Kir), CD161 (NK-1.1), CD244 (2B4), CD314 (NKG2D), CD319 (CRACC), CD328 (Siglec-7), CD335 (NKp46), Ly49, and Ly108. For NKT cells, exemplary extracellular markers can include, but are not limited to, the same markers as for NK cells, as well as CD3 and subunits of invariant TCRα including Vα24 and Jα18 TCR (iNKT). In some embodiments, the specificity of the recombinant antibodies as described herein can be for a molecular marker expressed on a dendritic cell. Non-limiting examples of dendritic cell markers include CD1C, CD8, CD11C, CD24, CD123, CD141, Necl-2, CD11c, HLADR, and BDCA3. Additional dendritic cell markers suitable for the systems and methods disclosed herein can be found in, for example, Villani et al., Science, 21 Apr. 2017: Vol. 356, Issue 6335.

### Tumor-Cell Marker Antibodies

Tumor cell markers that can be used include any marker that is expressed on tumors. In some aspects, the tumor cell markers can be a marker that is expressed more on cancerous cells, such as tumors, at a higher level than on non-cancerous cells. Exemplary markers include, but are not limited to, ALK, alpha-fetoprotein (AFP), beta-2-microglobulin (B2M), beta-human chorionic gonadotropin (Beta-hCG), bladder tumor antigen (BTA), BRCA1, BRCA2, BCR-ABL fusion gene (Philadelphia chromosome), BRAF V600 mutations, C-kit/CD117, CA15-3/CA27.29, CA-125, CA 27.29, carcinoembryonic antigen (CEA), CD20, CD22, CD25, CD30, CD31, CD33, CD44, CD133, CD176, CD276, estrogen receptor (ER), E-cadherin, ESPR, EGFR, EPCAM, GD2, progesterone receptor (PR), fibrin/fibrinogen, HE4 gene variants, HER2 gene variants, JAK2 gene variants, KRAS gene variants, nuclear matrix protein 22, PCA3, PML/RARα fusion gene, programmed death-ligand 1 (PD-L1 or CD274), prostate-specific antigen (PSA), TEM7, TEM8, and VEGF receptor family members.

### D. Methods for Identifying Tumor-Specific Antibodies

In one aspect, provided herein are methods for identifying a tumor-specific antibody, the method including: (a) partitioning a single B cell of a plurality of single B cells obtained from a first tumor sample in a partition of a first plurality of partitions; (b) determining all or a part of the nucleic acid sequences encoding one or more antibodies produced by the partitioned single B cell; (c) using the determined nucleic acid sequences to produce a recombinant antibody; (d) coupling the recombinant antibody to a reporter oligonucleotide including a reporter barcode sequence to generate a barcoded recombinant antibody; and (e) contacting the barcoded recombinant antibody with a second tumor sample, and identifying the recombinant antibody as an antibody having specificity for the second tumor sample if the barcoded recombinant antibody is capable of binding to an antigen associated with the tumor sample.

In some embodiments, the identifying of the produced antibody as an antibody having specificity for the second tumor sample further including quantifying levels of gene expression and/or protein marker expression in the single tumor cell, which can include the functional characteristics (e.g., the transcriptomic or proteomic) of the B cells and/or tumor cells associated with the recombinant antibodies. These functional characteristics can include transcription of cytokine, chemokine, or cell-surface associated molecules, such as, costimulatory molecules, checkpoint inhibitors, cell surface maturation markers, or cell-adhesion molecules. Such analysis allows a B cell or B-cell population expressing a given antibody to be associated with certain functional characteristics.

In some embodiments, the method further includes using the quantified levels for identification of biomarkers specific for the second tumor sample and/or a subject from whom the second tumor sample is obtained. In some embodiments, the method further includes quantifying binding affinity of the one or more therapeutic antibodies to the single tumor cells, for example by measuring the number of tumor cells that express at least one antigen that binds to the one or more therapeutic antibodies.

In some embodiments, the method further includes using the quantified binding affinity as an indicator of efficacy of treating a tumor with the one or more therapeutic antibodies. In some embodiments, the method further includes using the quantified binding affinity to monitor antigen escape of a tumor from the one or more therapeutic antibodies over time.

In some embodiments, the identifying of the produced antibody as an antibody having specificity for the second tumor sample further including comparing the determined nucleic acid sequences encoding the antibody to a genomic DNA sequence from the second tumor sample to confirm antigen specificity of the antibody. In some embodiments, the genomic DNA sequence is obtained from a single cell in the second tumor sample. In some embodiments, the genomic DNA sequence is obtained from a plurality of cells in the second tumor sample. In some embodiments, the genomic DNA sequence is obtained by whole-genome sequencing.

In some embodiments, the identifying of the produced antibody as an antibody having specificity for the second tumor sample further includes comparing the determined nucleic acid sequences encoding the barcoded antibody to a sequence of a ribonucleic acid (RNA) molecule from the second tumor sample to confirm antigen specificity of the antibody.

In some embodiments, the RNA molecule is obtained from a single cell in the second tumor sample. In some embodiments, the RNA molecule is obtained from a plurality of cells in the second tumor sample. In some embodiments, the method further includes obtaining the sequence of the RNA molecule. In some embodiments, the method further includes determining a nucleic acid sequence of a messenger RNA (mRNA) molecule from the single B cell and/or from the single tumor cell.

In some embodiments, the one or more nucleic acid barcode molecules includes one or more barcode sequences and the cDNAs resulting from the reverse transcription step will contain one or more barcode sequences corresponding to the barcode sequences of the nucleic acid barcode molecules. In some embodiments, the synthesis of complementary DNA includes barcoded reverse transcription carried out with in-droplet single cells.

In some embodiments, the determination of the mRNA sequences and the complementary DNA (cDNA) sequences includes whole transcriptome sequencing (e.g., whole-exome sequencing). In some embodiments, the determination of the mRNA sequences and the complementary DNA (cDNA) sequences includes next-generation sequencing (NGS).

In some embodiments, the method further includes administering the recombinant antibody to a subject in need thereof. In some embodiments, the method further includes administering an immune cell expressing the recombinant antibody to a subject in needed thereof. In some embodiments, the method further includes comparing the determined nucleic acid sequence of the recombinant antibody to sequences of known antibodies in order to identify the antibody as a tumor-specific antibody.

In some embodiments, the method further includes using a filter that takes into account clonal expansions to identify the antibody as a tumor-specific antibody. For example, from a set of antibodies, downselection can be performed by combining 1 or more of the following filters: (1) Retain cells with clonally related sequences observed more than once (filter out antibody lineages only present in a single cell). (2) Retain cells with a given isotype, e.g. IGHG3, which has enhanced complement deposition activity against a target-expressing cell. (3) Retain cells with antibodies that are 5%, 10%, 15%, or 20% mutated from germline (indicates that repeated antigen stimulation/binding has occurred).

In some embodiments, the methods of the disclosure further include using a filter that takes into account gene expression profiles of the single B cell to identify the antibody as a tumor-specific antibody. An exemplary method may comprise classifying B cells within a sample as naïve, transitional memory, class-switched memory, plasmablast, or plasma cell, and filtering for or selecting antibodies only present in memory, class-switched memory, or plasma cells.

In some embodiments, the determined nucleic acid sequence of the recombinant antibody can be used to generate an immune receptor, such as a chimeric antigen receptor (CAR). For example, the determined nucleic acid sequence of the recombinant antibody can be used to generate an antigen-specific receptor, e.g., a receptor that can immunologically recognize and/or specifically bind to an antigen, or an epitope thereof, such that binding of the antigen-specific receptor to antigen, or the epitope thereof, elicits an immune response. In some embodiments, the antigen-specific receptor has antigenic specificity for a cancer antigen, such as a tumor-specific antigen (TSA) or a tumor-associated antigen (TAA).

In some embodiments, the antigen-specific receptor is a chimeric antigen receptor (CAR). Generally, a CAR includes an antigen binding domain, e.g., a single-chain variable fragment (scFv) of an antibody, fused to a transmembrane domain and an intracellular domain. In this case, the antigenic specificity of a CAR can be encoded by a scFv which specifically binds to the antigen, or an epitope thereof. CARs, and methods of making them, are known in the art.

### E. Methods for Characterizing Antibody Specificity or Target Specificity

A particular valuable application of the methods and compositions described herein is in the characterization of antibody specificity and target specificity. Accordingly, some embodiments of the disclosure relate to methods for characterizing antibody specificity or target specificity. As described in greater detail in Example 1, the methods can begin by partitioning at least one single B cell of a plurality of single B cells obtained from a first tumor sample into individual partitions. The partitioning of the B cells are followed by a determination of the nucleic acid sequences encoding V_{H} and V_{L} regions of one or more antibodies produced by the partitioned single B cell. Subsequently, the determined nucleic acid sequences are used to produce recombinant antibodies that are determined above as being expressed in the original B cell. In some embodiments, for subsequent characterization and validation of the phenotypic properties of the produced recombinant antibodies, the produced recombinant antibodies are then coupled to a reporter oligonucleotide including a reporter barcode sequence to generate barcoded recombinant antibodies.

In some embodiment, the methods of the disclosure include a step of selecting B-cell derived antibodies suitable for production of recombinant antibodies and optionally for production of barcoded recombinant antibodies. A non-limiting exemplary approach suitable for antibody selection includes the comparative analysis of antibody repertoires between the tumor sample and one or both of (i) normal associated tissue, and (ii) peripheral blood. These comparator populations can be sequenced, for example, by using bulk sequencing to reduce cost and to gain a larger number of sequences. This comparative analysis can enable one to identify antibodies that are enriched within the tumor compared with other compartments. Another non-limiting approach suitable for antibody selection includes quantification of the amount of somatic hypermutation that has occurred within the potential antibody candidates by comparing against either a reference genome or the donor's own germline sequences. Antibodies with the largest amount of somatic hypermutations (SHM) can be selected based on the assumption that they will have already been selected to be high affinity. In addition, antibodies with the least affinity on the assumption that they are novel infiltrating cells with new specificity. The above approaches of antibody selection can be employed individually or in combination.

In some embodiments, the barcoded recombinant antibodies generated as described above are then contacted with a population of tumor single cells (e.g., a dissociated tumor sample, a single cell suspension of tumor cells) derived from a second tumor sample, followed by identification of one or more recombinant antibodies having specificity for the second tumor sample, as indicated by the ability of the corresponding barcoded recombinant antibodies to bind to an antigen associated with the second tumor sample.

In some embodiments, gene expression and protein marker expression analyses are additionally performed on (1) the tumor sample from which the B cell is derived, and (2) the tumor sample from which the V_{H} and V_{L} mRNAs are derived. In some embodiments, comparative analysis of the gene expression and protein marker expression datasets from to (1) and (2) is subsequently performed to determine the recombinant antibodies' specificity and target specificity.

### F. Methods for Enhanced Identification of Patient-Specific or Population-Specific Biomarkers

In some embodiments, the methods, compositions and systems disclosed herein are utilized to enhance the identification of patient-specific or population-specific biomarkers on circulating tumor cells. As described in greater detail in Example 2, the methods begin by partitioning at least one single B cell of a plurality of single B cells obtained from a first tumor sample into individual partitions. The partitioning of the B cells are followed by a determination of the nucleic acid sequences encoding V_{H} and V_{L} regions of one or more antibodies produced by the partitioned single B cell. Subsequently, the determined nucleic acid sequences are used to produce recombinant antibodies that are determined above as being expressed in the original B cell. In some embodiments, for subsequent characterization and validation of the phenotypic properties of the produced recombinant antibodies, the produced recombinant antibodies are then coupled to a reporter oligonucleotide including a reporter barcode sequence to generate barcoded recombinant antibodies.

In some embodiments, the barcoded recombinant antibodies generated as described above are then contacted with a population of tumor single cells (e.g., a dissociated tumor sample, a single cell suspension of tumor cells) derived from a second tumor sample, followed by identification of one or more recombinant antibodies having specificity for the second tumor sample, as indicated by the ability of the corresponding barcoded recombinant antibodies to bind to an antigen associated with the second tumor sample.

In some embodiments, comparative analysis of in vitro and/or in vivo characterization the barcoded recombinant antibodies as well as gene expression and protein marker expression analysis of a population of tumor samples are subsequently performed to identify biomarkers specific for individual tumor sample or for a population of tumor samples.

G. Methods for Monitoring Antigen Escape in an Individual Who has been Treated with an Antibody-Based Therapy

In another aspect, some embodiments of the disclosure relate to methods for monitoring antigen escape in an individual who has been treated with an antibody-based therapy. As described in greater detail in Example 3, the methods begin by partitioning at least one single B cell of a plurality of single B cells obtained from a first tumor sample into individual partitions. The partitioning of the B cells are followed by a determination of the nucleic acid sequences encoding V_{H} and V_{L} regions of one or more antibodies produced by the partitioned single B cell. Subsequently, the determined nucleic acid sequences are used to produce recombinant antibodies that are determined above as being expressed in the original B cell. In some embodiments, for subsequent characterization and validation of the phenotypic properties of the produced recombinant antibodies, the produced recombinant antibodies are then coupled to a reporter oligonucleotide including a reporter barcode sequence to generate barcoded recombinant antibodies.

In some embodiments, the barcoded recombinant antibodies generated as described above are then contacted with a population of tumor single cells (e.g., a dissociated tumor sample, a single cell suspension of tumor cells) derived from a second tumor sample, followed by identification of one or more recombinant antibodies having specificity for the second tumor sample, as indicated by the ability of the corresponding barcoded recombinant antibodies to bind to a tumor cell of the second tumor sample and/or an antigen associated with the second tumor sample. Tumor cells in the second tumor sample can be identified and/or enriched using antibodies specific for one or more tumor-cell markers, e.g., those expressed more on cancerous cells at a higher level than on non-cancerous cells. Suitable antibodies include, but are not limited to, those specific for ALK, alpha-fetoprotein (AFP), beta-2-microglobulin (B2M), beta-human chorionic gonadotropin (Beta-hCG), bladder tumor antigen (BTA), BRCA1, BRCA2, BCR-ABL fusion gene (Philadelphia chromosome), BRAF V600 mutations, C-kit/CD117, CA15-3/CA27.29, CA-125, CA 27.29, carcinoembryonic antigen (CEA), CD20, CD22, CD25, CD30, CD31, CD33, CD44, CD133, CD176, CD276, estrogen receptor (ER), E-cadherin, ESPR, EGFR, EPCAM, GD2, progesterone receptor (PR), fibrin/fibrinogen, HE4 gene variants, HER2 gene variants, JAK2 gene variants, KRAS gene variants, nuclear matrix protein 22, PCA3, PML/RARα fusion gene, programmed death-ligand 1 (PD-L1 or CD274), prostate-specific antigen (PSA), TEM7, TEM8, and VEGF receptor family members.

In some embodiments, tumor cells in the second tumor sample can be identified by using unbiased genome-wide sequence analysis or whole transcriptome gene expression profiling of the cells for cancer-related mRNAs. In some embodiments, tumor cells in the second tumor sample can be identified using targeted gene expression profiling of the cells for cancer-related mRNAs. In some embodiments, whole transcriptome libraries are selectively enriched for cancer-related transcripts and the enriched libraries subjected to sequencing. Approaches, systems, and kits suitable for use in targeted characterization and enrichment of cancer-related transcripts are known in the art and/or commercially available. For example, in some embodiments, whole transcriptome libraries can be selectively enriched for cancer-related transcripts by using 10× Genomics Human Pan-Caner Panel kit (Cat #PN-1000247 and PN-1000260) with reagents for use in targeted gene expression analysis of >1,200 cancer-related biomarkers to identify, characterize, enrich, and/or profile a pre-designed set of transcripts for a target cancer of interest.

In some embodiments, the binding affinity of the barcoded recombinant antibody to a tumor sample is subsequently evaluated by measuring the number of tumor cells expressing a target antigen of the barcoded recombinant antibody that are capable to binding to the barcoded recombinant antibody. In these experiments, the quantified binding affinity of the barcoded recombinant antibody to the tumor sample is indicative of the therapeutic antibody's efficacy in treating the tumor.

### Temporal Analysis

In some embodiments, the binding affinity of the barcoded recombinant antibody to an antigen expressed by the tumor sample is monitored over time, and is used as an indication of antigen escape from the recombinant antibody over time (e.g., before or after treatment with an therapeutic agent or different stages of differentiation). In some examples, the methods described herein can be performed on multiple similar tumor samples or tumor cells obtained from the same subject at a different time points (e.g., before or after treatment with a therapeutic agent, different stages of differentiation, different stages of disease progression, different ages of the subject, or before or after development of resistance to a therapeutic agent).

In some embodiments, the methods described herein can be performed on multiple similar tumor samples or tumor cells obtained from the subject at 2, 3, 4, 5, 6, 7, 8, 9, 10, or more times. For example, the multiple similar tumor samples can be repetitive samples from the same subject, the same tissue, the same organoid, the same cell suspension, or any other biological sample. In some embodiments, the same tumor sample or tumor cell is contacted with different barcoded recombinant antibodies at each time point. In some embodiments, samples can be obtained from the same subject as a routine monitoring on a monthly basis, or at the shortest time interval 10-14 days. This timeline can be used when monitoring for de novo immune responses against the developed antibody therapeutics. Monthly monitoring can also be used for circulating tumor cell content and ctDNA.

### H. Methods for Characterizing a Potential Antigen

In another aspect, some embodiments of the disclosure relate to methods for characterizing a potential antigen for an antibody or fragment thereof. As described in greater detail in Example 4, the methods begin by partitioning at least one single B cell of a plurality of single B cells obtained from a first tumor sample into individual partitions. The partitioning of the B cells are followed by a determination of the nucleic acid sequences encoding V_{H} and V_{L} regions of one or more antibodies produced by the partitioned single B cell. Subsequently, the determined nucleic acid sequences are used to produce a recombinant antibody that is determined above as being expressed in the original B cell. In some embodiments, for subsequent characterization and validation of the phenotypic properties of the produced recombinant antibodies, one or more produced recombinant antibodies from one or more B cells obtained from the first tumor sample are then coupled to a reporter oligonucleotide including a reporter barcode sequence to generate a set of one or more barcoded recombinant antibodies. In some embodiments, variants, e.g., mutants, of the individual antibodies can also be used as part of the set to identify paratopes/residues on the antibody required for antigen recognition. Vice versa, in some embodiments, variants of the target proteins used for epitopes/recognized portions of the antigen.

In some embodiments, the binding affinity of the barcoded recombinant antibodies to a second tumor sample is subsequently evaluated by measuring the number of tumor cells expressing a target antigen of the barcoded recombinant antibodies that are capable to binding to the barcoded recombinant antibodies, followed by using the quantified binding affinity to determine if the recombinant antibodies compete with one another for binding to the second tumor sample. In this case, the second tumor sample or cells of the second tumor are known to express a particular target antigen of interest, or else the recombinant antibody from the B cell above is thought to bind to a particular target antigen.

In some embodiments, barcoded recombinant antibodies from the set are indicated as competing for binding to an antigen if it is determined that they bind to different cells in the second tumor sample in a mutually exclusive manner. For example, competitive binding assays can be perform to identify mutually exclusive detection of antibodies that bind to different cells in a tumor population. In addition or alternatively, binding assays can be performed on cell replicates with differing concentrations of barcoded antibodies and detect tighter binding of the antibodies versus each other. In some embodiments, dose response curves, wherein cells of the second tumor sample are contacted with varying concentrations one or more barcoded recombinant antibodies of the set, are used to evaluate whether the barcoded recombinant antibodies of the set compete for binding. For example, in dose response curve varying the concentrations of both antibodies, if increasing dose of one antibody results in less binding to the other, this may indicate a level of competition between the two tested antibodies. In some embodiments, the quantified binding affinity of the recombinant antibodies are also co-associated with RNA expression analysis to identify potential antigen.

### I. Methods of Treatment

In some embodiments, the methods of the disclosure further include administering a therapeutic composition including a recombinant antibody as described herein and/or an immune system cell expressing the recombinant antibody as described herein to a subject in need thereof. Non-limiting examples of immune system cells include B cells, monocytes, NK cells, natural killer T (NKT) cells, basophil, eosinophil, neutrophil, dendritic cells, macrophages, regulatory T cells, helper T cells (T_{H}), cytotoxic T cells (T_{CTL}), memory T cells, gamma delta (γδ) T cells, hematopoietic stem cells, and hematopoietic stem cell progenitors. In some embodiments, the immune system cell is a T cell. In some embodiments, the therapeutic composition is formulated to be compatible with its intended route of administration. For example, the recombinant antibodies of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject recombinant antibodies of the disclosure can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

For example, the data obtained from the cell culture assays and animal studies can be used in formulating a range of dosage for use in humans. The dosage of such compounds lies generally within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

### J. Systems and Methods for Partitioning

In some aspects, such as those that have been described above, the methods provided herein include a step of partitioning or may include an additional processing step(s). This description sets forth examples, embodiments and characteristics of steps of the methods and of reagents useful in the methods or as may be provided in the partitions.

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions.

In some embodiments disclosed herein, the partitioned particle (e.g., biological particle), is a cell of B cell lineage (e.g., labelled cell of B-cell lineage), e.g. a plasma cell or memory B cell, which expresses an antigen-binding molecule (e.g., an immune receptor, an antibody or a functional fragment thereof) on its surface. In other examples, the partitioned particle can be a cell (e.g., labelled cell) engineered to express antigen-binding molecules (e.g., an immune receptors, antibodies or functional fragments thereof).

In some embodiments, the partitioned biological particle is a cell of T cell lineage, which expresses an antigen-binding molecule, e.g., a TCR.

In some embodiments, the partitioned biological particle is obtained from a tumor sample.

The term “partition,” as used herein, generally, refers to a space or volume that can be suitable to contain one or more cells, one or more species of features or compounds, or conduct one or more reactions. A partition can be a physical container, compartment, or vessel, such as a droplet, a flowcell, a reaction chamber, a reaction compartment, a tube, a well, or a microwell. In some embodiments, the compartments or partitions include partitions that are flowable within fluid streams. These partitions can include, for example, micro-vesicles that have an outer barrier surrounding an inner fluid center or core, or, in some cases, the partitions can include a porous matrix that is capable of entraining and/or retaining materials within its matrix. In some aspects, partitions comprise droplets of aqueous fluid within a non-aqueous continuous phase (e.g., oil phase). A variety of different vessels are described in, for example, U.S. Patent Application Publication No. 2014/0155295. Emulsion systems for creating stable droplets in non-aqueous or oil continuous phases are described in detail in, e.g., U.S. Patent Application Publication No. 2010/010511.

In some embodiments, a partition herein includes a space or volume that can be suitable to contain one or more species or conduct one or more reactions. A partition can be a physical compartment, such as a droplet or well. The partition can be an isolated space or volume from another space or volume. The droplet can be a first phase (e.g., aqueous phase) in a second phase (e.g., oil) immiscible with the first phase. The droplet can be a first phase in a second phase that does not phase separate from the first phase, such as, for example, a capsule or liposome in an aqueous phase. A partition can include one or more other (inner) partitions. In some cases, a partition can be a virtual compartment that can be defined and identified by an index (e.g., indexed libraries) across multiple and/or remote physical compartments. For example, a physical compartment can include a plurality of virtual compartments.

In some embodiments, the methods and systems described herein provide for the compartmentalization, depositing or partitioning of individual cells from a sample material containing cells (e.g., after at least one labelling agent or reporter agent molecule has been bound to a cell surface feature of a cell) into discrete partitions, where each partition maintains separation of its own contents from the contents of other partitions. Identifiers including unique identifiers (e.g., UMI) and common or universal tags, e.g., barcodes, can be previously, subsequently or concurrently delivered to the partitions that hold the compartmentalized or partitioned cells, in order to allow for the later attribution of the characteristics of the individual cells to one or more particular compartments. Further, identifiers including unique identifiers and common or universal tags, e.g., barcodes, can be coupled to labelling agents and previously, subsequently or concurrently delivered to the partitions that hold the compartmentalized or partitioned cells, in order to allow for the later attribution of the characteristics of the individual cells to one or more particular compartments. Identifiers including unique identifiers and common or universal tags, e.g., barcodes, can be delivered, for example on an oligonucleotide, to a partition via any suitable mechanism, for example by coupling the barcoded oligonucleotides to a microcapsule (e.g., bead). In some embodiments, the barcoded oligonucleotides are reversibly (e.g., releasably) coupled to a microcapsule (e.g., bead). The microcapsule (e.g., bead) suitable for the compositions and methods of the disclosure can have different surface chemistries and/or physical volumes. In some embodiments, the microcapsule (e.g., bead) includes a polymer gel. In some embodiments, the polymer gel is a polyacrylamide. Additional non-limiting examples of suitable microcapsules (e.g., beads) include microparticles, nanoparticles, beads, and microbeads. In some embodiments, the microcapsule includes a bead. The partition can be a droplet in an emulsion. A partition can include one or more particles. A partition can include one or more types of particles. For example, a partition of the present disclosure can include one or more biological particles, e.g., cells and/or macromolecular constituents thereof, e.g., B cells, labelled B cells such as memory B cells or plasma cells. A partition can include one or more gel beads. A partition can include one or more cell beads. A partition can include a single gel bead, a single cell bead, or both a single cell bead and single gel bead. A partition can include one or more reagents. Alternatively, a partition can be unoccupied. For example, a partition cannot comprise a bead. Unique identifiers, such as barcodes, can be injected into the droplets previous to, subsequent to, or concurrently with droplet generation, such as via a microcapsule (e.g., bead), as described elsewhere herein. Microfluidic channel networks (e.g., on a chip) can be utilized to generate partitions as described herein. Alternative mechanisms can also be employed in the partitioning of individual biological particles, including porous membranes through which aqueous mixtures of cells are extruded into non-aqueous fluids.

The partitions can be flowable within fluid streams. The partitions can include, for example, micro-vesicles that have an outer barrier surrounding an inner fluid center or core. In some cases, the partitions can include a porous matrix that is capable of entraining and/or retaining materials (e.g., expressed antibodies or antigen binding fragments thereof) within its matrix (e.g., via a capture agent configured to couple to both the matrix and the expressed antibody or antigen binding fragment thereof). The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions can be provided in a water-in-oil emulsion or oil-in-water emulsion. A variety of different vessels is described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In the case of droplets in an emulsion, allocating individual particles (e.g., biological particles, e.g., cells, labelled B cells, memory cells, plasma cells) to discrete partitions can, in one non-limiting example, be accomplished by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters can be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions can contain less than one biological particle per partition in order to ensure that those partitions that are occupied are primarily singly occupied. In some cases, partitions among a plurality of partitions can contain at most one biological particle (e.g., bead, DNA, cell, such as a labelled B cell or plasma cell, cellular organelle, or cellular material). In some embodiments, the various parameters (e.g., fluid properties, particle properties, microfluidic architectures, etc.) can be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

In some embodiments, the method further includes individually partitioning one or more single cells (e.g., one or more single tumor cells) from a plurality of cells (e.g., from the second tumor samples) in a partition of a second plurality of partitions.

In some embodiments, at least one of the first and second plurality of partitions includes a microwell, a flowcell, a reaction chamber, a reaction compartment, or a droplet. In some embodiments, at least one of the first and second plurality of partitions includes individual droplets in emulsion. In some embodiments, the partitions of the first plurality and/or the second plurality of partition have the same reaction volume.

In the case of droplets in emulsion, allocating individual cells to discrete partitions can generally be accomplished by introducing a flowing stream of cells in an aqueous fluid into a flowing stream of a non-aqueous fluid, such that droplets are generated at the junction of the two streams. By providing the aqueous cell-containing stream at a certain concentration of cells, the occupancy of the resulting partitions (e.g., number of cells per partition) can be controlled. For example, where single cell partitions are desired, the relative flow rates of the fluids can be selected such that, on average, the partitions contain less than one cell per partition, in order to ensure that those partitions that are occupied, are primarily singly occupied. In some embodiments, the relative flow rates of the fluids can be selected such that a majority of partitions are occupied, e.g., allowing for only a small percentage of unoccupied partitions. In some embodiments, the flows and channel architectures are controlled as to ensure a desired number of singly occupied partitions, less than a certain level of unoccupied partitions and less than a certain level of multiply occupied partitions.

In some embodiments, the methods described herein can be performed such that a majority of occupied partitions include no more than one cell per occupied partition. In some embodiments, the partitioning process is performed such that fewer than 25%, fewer than 20%, fewer than 15%, fewer than 10%, fewer than 5%, fewer than 2%, or fewer than 1% the occupied partitions contain more than one cell. In some embodiments, fewer than 20% of the occupied partitions include more than one cell. In some embodiments, fewer than 10% of the occupied partitions include more than one cell per partition. In some embodiments, fewer than 5% of the occupied partitions include more than one cell per partition. In some embodiments, it is desirable to avoid the creation of excessive numbers of empty partitions. For example, from a cost perspective and/or efficiency perspective, it may be desirable to minimize the number of empty partitions. While this can be accomplished by providing sufficient numbers of cells into the partitioning zone, the Poissonian distribution can optionally be used to increase the number of partitions that include multiple cells. As such, in some embodiments described herein, the flow of one or more of the cells, or other fluids directed into the partitioning zone are performed such that no more than 50% of the generated partitions, no more than 25% of the generated partitions, or no more than 10% of the generated partitions are unoccupied. Further, in some aspects, these flows are controlled so as to present non-Poissonian distribution of single occupied partitions while providing lower levels of unoccupied partitions. Restated, in some aspects, the above noted ranges of unoccupied partitions can be achieved while still providing any of the single occupancy rates described above. For example, in some embodiments, the use of the systems and methods described herein creates resulting partitions that have multiple occupancy rates of less than 25%, less than 20%, less than 15%), less than 10%, and in some embodiments, less than 5%, while having unoccupied partitions of less than 50%), less than 40%, less than 30%, less than 20%, less than 10%, and in some embodiments, less than 5%.

Although described in terms of providing substantially singly occupied partitions, above, in some embodiments, the methods as described herein include providing multiply occupied partitions, e.g., containing two, three, four or more cells and/or microcapsules (e.g., beads) comprising nucleic acid barcode molecules within a single partition.

In some embodiments, the reporter oligonucleotides contained within a partition are distinguishable from the reporter oligonucleotides contained within other partitions of the plurality of partitions. This can be accomplished by incorporating one or more partition-specific barcode sequences into the reporter barcode sequence of the reporter oligonucleotides contained within the partition.

In some embodiments, it may be desirable to incorporate multiple different barcode sequences within a given partition, either attached to a single or multiple beads within the partition. For example, in some cases, a mixed, but known barcode sequences set can provide greater assurance of identification in the subsequent processing, e.g., by providing a stronger address or attribution of the barcodes to a given partition, as a duplicate or independent confirmation of the output from a given partition.

Microfluidic Channel Structures

Microfluidic channel networks (e.g., on a chip) can be utilized to generate partitions as described herein. Alternative mechanisms can also be employed in the partitioning of individual biological particles, including porous membranes through which aqueous mixtures of cells are extruded into non-aqueous fluids.

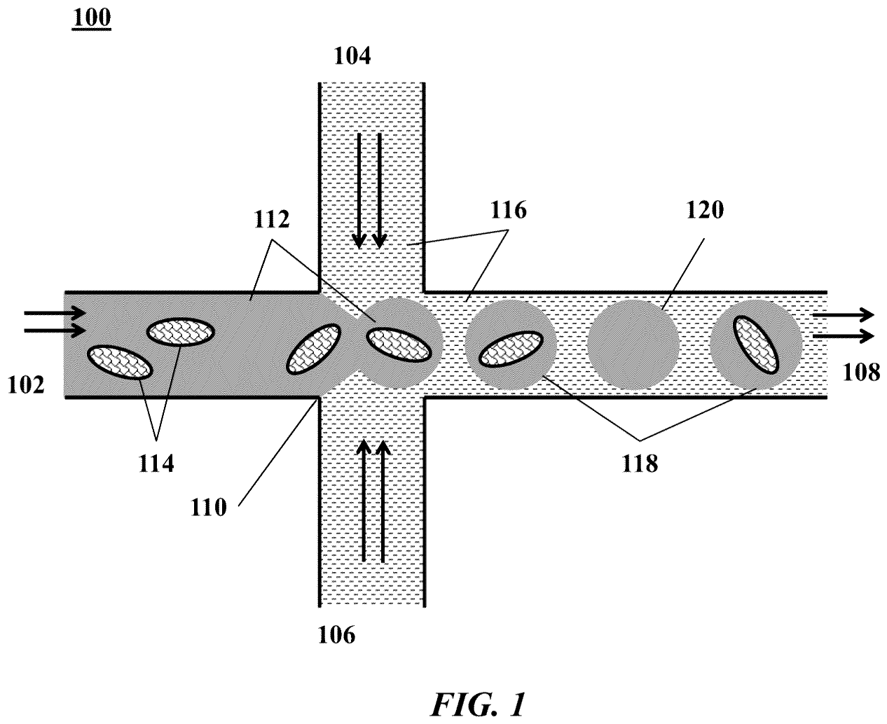

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (e.g., cells, for example, engineered cells, B cells, labelled B cells, plasma cells, memory B cells, or T cells) 114 can be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 can be fluidically coupled to an outlet reservoir where the discrete droplets can be stored and/or harvested. A discrete droplet generated can include an individual biological particle 114 (such as droplets 118). A discrete droplet generated can include more than one individual biological particle (e.g., B cells, B cells, labelled B cells, memory B cells, plasma cells, or labeled T cells) 114 (not shown in FIG. 1). A discrete droplet can contain no biological particle 114 (such as droplet 120). Each discrete partition can maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein can be coupled to any of a variety of different fluid sources or receiving components, including reservoirs, tubing, manifolds, or fluidic components of other systems. As will be appreciated, the microfluidic channel structure 100 can have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid can be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

The generated droplets can include two subsets of droplets: (1) occupied droplets 118, containing one or more biological particles 114, e.g., cells, B cells, labelled B cells, memory B cells, T cells, or plasma cells, and (2) unoccupied droplets 120, not containing any biological particles 114. Occupied droplets 118 can include singly occupied droplets (having one biological particle, such as one B cell, plasma cells, memory B cell, or T cell) and multiply occupied droplets (having more than one biological particle, such as multiple B cells, plasma cells, memory B cells, or T cells). As described elsewhere herein, in some cases, the majority of occupied partitions can include no more than one biological particle, e.g., cell, B cell, labelled B cell, memory B cell, plasma cell, per occupied partition and some of the generated partitions can be unoccupied (of any biological particle, e.g., cell). In some cases, though, some of the occupied partitions can include more than one biological particle, e.g., labelled B cell or plasma cell. In some cases, the partitioning process can be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it can be desirable to minimize the creation of excessive numbers of empty partitions, such as to reduce costs and/or increase efficiency. While this minimization can be achieved by providing a sufficient number of biological particles (e.g., cells, B cells, labelled B cells, plasma cells, memory B cells, or T cells 114) at the partitioning junction 110, such as to ensure that at least one biological particle is encapsulated in a partition, the Poissonian distribution can expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles, such as B cells, plasma cells, or T cells, (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

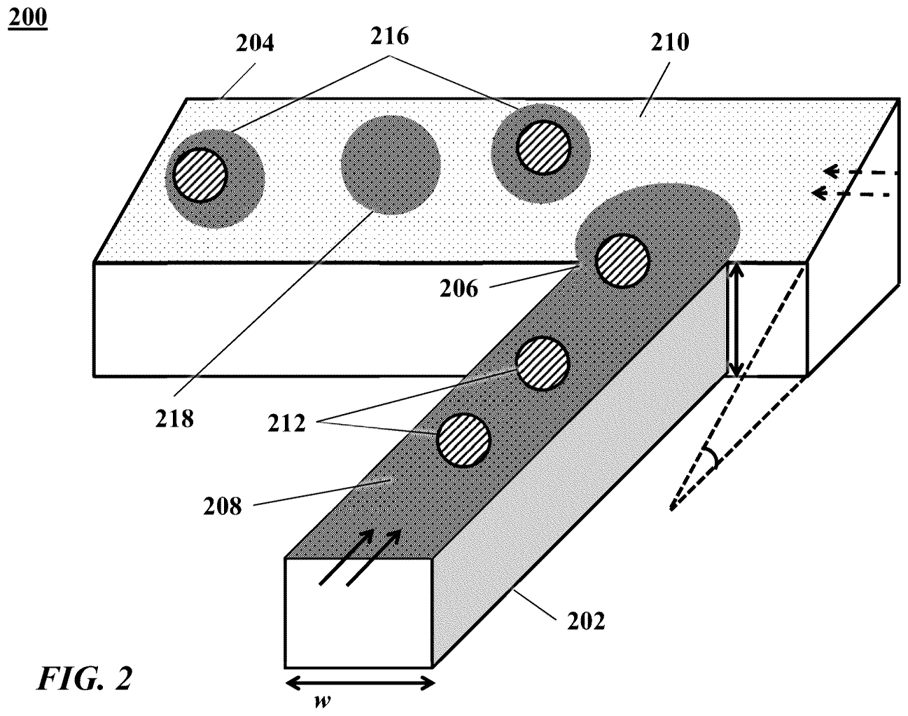

As will be appreciated, the above-described occupancy rates are also applicable to partitions that include both biological particles (e.g., cells, B cells, labelled B cells, memory B cells, or plasma cells) and additional reagents, including, but not limited to, microcapsules or beads (e.g., gel beads) carrying nucleic acid barcode molecules (e.g., barcoded oligonucleotides, barcoded nucleic acid molecules) (described in relation to FIGS. 1 and 2). The occupied partitions (e.g., at least about 10%, 20%, 30%, 40%, 50%, 60%, 70%, 80%, 90%, 95%, or 99% of the occupied partitions) can include both a microcapsule (e.g., bead) comprising nucleic acid barcode molecules (e.g., barcoded nucleic acid molecules) and a biological particle.

In another aspect, in addition to or as an alternative to droplet based partitioning, biological particles can be encapsulated within a microcapsule that comprises an outer shell, layer or porous matrix in which is entrained one or more individual biological particles or small groups of biological particles. The microcapsule can include other reagents. Encapsulation of biological particles, e.g., cells, B cells, labelled B cells, memory B cells, or plasma cells, can be performed by a variety of processes. Such processes can combine an aqueous fluid containing the biological particles with a polymeric precursor material that can be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

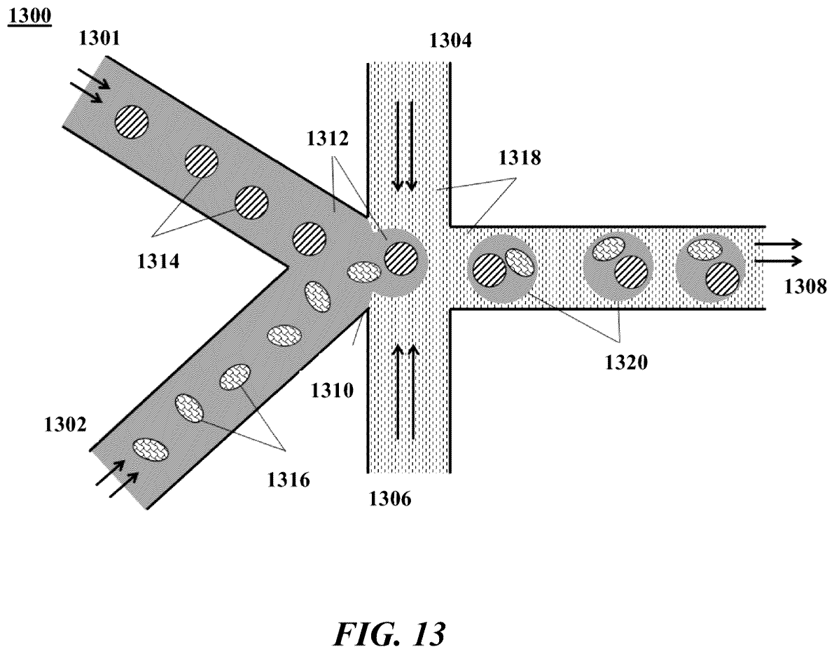

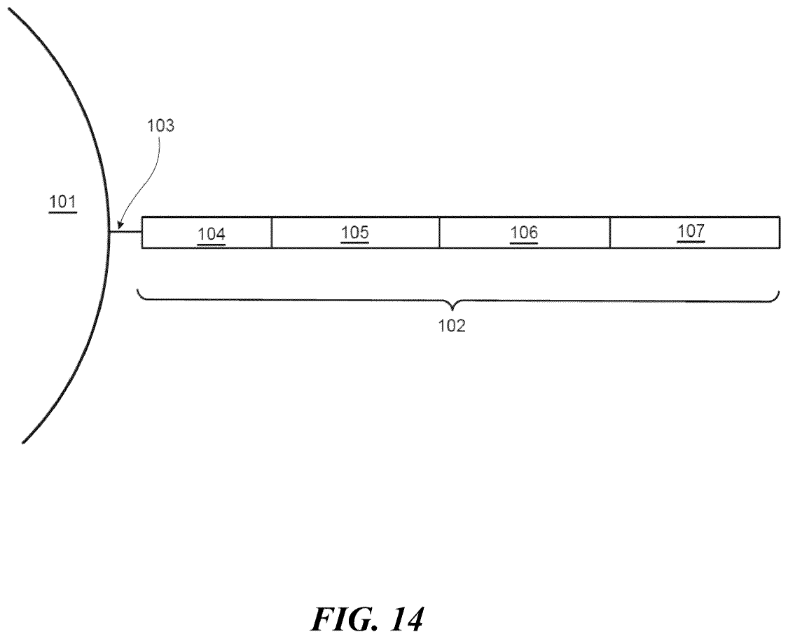

FIG. 13 shows an example of a microfluidic channel structure 1300 for delivering barcode carrying beads to droplets. The channel structure 1300 can include channel segments 1301, 1302, 1304, 1306 and 1308 communicating at a channel junction 1310. In operation, the channel segment 1301 may transport an aqueous fluid 1312 that includes a plurality of beads 1314 (e.g., with nucleic acid molecules, e.g., nucleic acid barcode molecules or barcoded oligonucleotides, molecular tags) along the channel segment 1301 into junction 1310. The plurality of beads 1314 may be sourced from a suspension of beads. For example, the channel segment 1401 may be connected to a reservoir comprising an aqueous suspension of beads 1314. The channel segment 1302 may transport the aqueous fluid 1312 that includes a plurality of biological particles 1316 along the channel segment 1302 into junction 1310. The plurality of biological particles 1316 may be sourced from a suspension of biological particles. For example, the channel segment 1302 may be connected to a reservoir comprising an aqueous suspension of biological particles 1316. In some instances, the aqueous fluid 1312 in either the first channel segment 1301 or the second channel segment 1302, or in both segments, can include one or more reagents, as further described below. A second fluid 1318 that is immiscible with the aqueous fluid 1312 (e.g., oil) can be delivered to the junction 1310 from each of channel segments 1304 and 1406. Upon meeting of the aqueous fluid 1312 from each of channel segments 1301 and 1402 and the second fluid 1318 from each of channel segments 1304 and 1306 at the channel junction 1310, the aqueous fluid 1312 can be partitioned as discrete droplets 1420 in the second fluid 1318 and flow away from the junction 1310 along channel segment 1308. The channel segment 1308 may deliver the discrete droplets to an outlet reservoir fluidly coupled to the channel segment 1308, where they may be harvested. As an alternative, the channel segments 1301 and 1302 may meet at another junction upstream of the junction 1310. At such junction, beads and biological particles may form a mixture that is directed along another channel to the junction 1310 to yield droplets 1420. The mixture may provide the beads and biological particles in an alternating fashion, such that, for example, a droplet comprises a single bead and a single biological particle.

In another aspect, in addition to or as an alternative to droplet-based partitioning, biological particles (e.g., cells) may be entrained within a particulate material to form a “cell bead”.

The cell bead can include other reagents. Formation of cell beads can be performed by a variety of processes. Such processes can combine an aqueous fluid containing the biological particles with a polymeric precursor material that can be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Formation of cell beads can be performed by a variety of methods. For example, air knife droplet or aerosol generators may be used to dispense droplets of precursor fluids into gelling solutions in order to form microcapsules, e.g., cell beads that include individual biological particles or small groups of biological particles (e.g., labelled B cells or plasma cells). Likewise, membrane-based encapsulation systems may be used to generate microcapsules (e.g., cell beads) comprising encapsulated biological particles (e.g., B cells or plasma cells) as described herein. Microfluidic systems of the present disclosure, such as that shown in FIG. 1, may be readily used in encapsulating cells or generating cell beads from biological particles (e.g., cells) as described herein. Exemplary methods for generating cell beads from biological particles (e.g., cells) are also further described in U.S. Patent Application Pub. No. US 2015/0376609 and PCT/US2018/016019. In particular, and with reference to FIG. 1, the aqueous fluid 112 comprising (i) the biological particles (e.g., labelled B cells or plasma cells) 114 and (ii) the polymer precursor material (not shown) is flowed into channel junction 110, where it is partitioned into droplets 118, 120 through the flow of non-aqueous fluid 116. In the case of encapsulation methods or methods for entraining biological particles in particulate material to form cell beads, non-aqueous fluid 116 may also include an initiator (not shown) to cause polymerization and/or crosslinking of the polymer precursor to form the microcapsule (e.g., bead) that includes the entrained biological particles. Examples of polymer precursor/initiator pairs include those described in U.S. Patent Application Publication No. 2014/0378345, which is entirely incorporated herein by reference for all purposes.

For example, in the case where the polymer precursor material comprises a linear polymer material, such as a linear polyacrylamide, PEG, or other linear polymeric material, the activation agent can include a cross-linking agent, or a chemical that activates a cross-linking agent within the formed droplets. Likewise, for polymer precursors that comprise polymerizable monomers, the activation agent can include a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) can be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED can diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) microcapsules, e.g., cell beads, as solid or semi-solid beads or particles entraining the cells (e.g., B cells, labelled B cells or plasma cells) 114. Although described in terms of polyacrylamide entrainment or encapsulation, other “activatable” entrainment or encapsulation compositions can also be employed in the context of the methods and compositions described herein. For example, formation of alginate droplets followed by exposure to divalent metal ions (e.g., Ca′ ions), can be used as an entrainment or encapsulation process using the described processes. Likewise, agarose droplets can also be transformed into capsules through temperature based gelling (e.g., upon cooling, etc.).

In some cases, entrained or encapsulated biological particles can be selectively releasable from the microcapsules or cell beads, such as through passage of time or upon application of a particular stimulus, that degrades the microcapsule or entraining material sufficiently to allow the biological particles (e.g., cells, B cells, labelled B cells, memory cells, or plasma cells, or macromolecular constituents thereof), or its other contents to be released from the microcapsule or entraining material, such as into a partition (e.g., droplet). For example, in the case of the polyacrylamide polymer described above, degradation of the polymer can be accomplished through the introduction of an appropriate reducing agent, such as DTT or the like, to cleave disulfide bonds that cross-link the polymer matrix. See, for example, U.S. Patent Application Publication No. 2014/0378345.

The biological particle (e.g., cell, B cell, labelled B cell, memory B cell, or plasma cell) can be subjected to other conditions sufficient to polymerize or gel the precursors. The conditions sufficient to polymerize or gel the precursors can include exposure to heating, cooling, electromagnetic radiation, and/or light. The conditions sufficient to polymerize or gel the precursors can include any conditions sufficient to polymerize or gel the precursors. Following polymerization or gelling, a polymer or gel can be formed around the biological particle (e.g., cell, B cell, labelled B cell, memory B cell, or plasma cell). The polymer or gel can be diffusively permeable to chemical or biochemical reagents. The polymer or gel can be diffusively impermeable to macromolecular constituents. In this manner, the polymer or gel can act to allow the biological particle (e.g., cell, B cell, labelled B cell, memory B cell, or plasma cell) to be subjected to chemical or biochemical operations while spatially confining the macromolecular constituents to a region of the droplet defined by the polymer or gel. The polymer or gel can include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel can include any other polymer or gel.

The polymer or gel can be functionalized (e.g., coupled to a capture agent) to bind to targeted analytes (e.g., secreted antibodies or antigen binding fragment thereof), such as nucleic acids, proteins, carbohydrates, lipids or other analytes. The polymer or gel can be polymerized or gelled via a passive mechanism. The polymer or gel can be stable in alkaline conditions or at elevated temperature. The polymer or gel can have mechanical properties similar to the mechanical properties of the bead. For instance, the polymer or gel can be of a similar size to the bead. The polymer or gel can have a mechanical strength (e.g. tensile strength) similar to that of the bead. The polymer or gel can be of a lower density than an oil. The polymer or gel can be of a density that is roughly similar to that of a buffer. The polymer or gel can have a tunable pore size. The pore size can be chosen to, for instance, retain denatured nucleic acids. The pore size can be chosen to maintain diffusive permeability to exogenous chemicals such as sodium hydroxide (NaOH) and/or endogenous chemicals such as inhibitors. The polymer or gel can be biocompatible. The polymer or gel can maintain or enhance cell viability. The polymer or gel can be biochemically compatible. The polymer or gel can be polymerized and/or depolymerized thermally, chemically, enzymatically, and/or optically.

The polymer can include poly(acrylamide-co-acrylic acid) crosslinked with disulfide linkages. The preparation of the polymer can include a two-step reaction. In the first activation step, poly(acrylamide-co-acrylic acid) can be exposed to an acylating agent to convert carboxylic acids to esters. For instance, the poly(acrylamide-co-acrylic acid) can be exposed to 4-(4,6-dimethoxy-1,3,5-triazin-2-yl)-4-methylmorpholinium chloride (DMTMM). The polyacrylamide-co-acrylic acid can be exposed to other salts of 4-(4,6-dimethoxy-1,3,5-triazin-2-yl)-4-methylmorpholinium. In the second cross-linking step, the ester formed in the first step can be exposed to a disulfide crosslinking agent. For instance, the ester can be exposed to cystamine (2,2′-dithiobis(ethylamine)). Following the two steps, the biological particle can be surrounded by polyacrylamide strands linked together by disulfide bridges. In this manner, the biological particle can be encased inside of or comprise a gel or matrix (e.g., polymer matrix) to form a “cell bead.” A cell bead can contain biological particles (e.g., cells, B cells, labelled B cells, memory B cells, or plasma cells) or macromolecular constituents (e.g., RNA, DNA, proteins, secreted antibodies or antigen binding fragments thereof etc.) of biological particles. A cell bead can include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both (i) cell beads (and/or droplets or other partitions) containing biological particles and (ii) cell beads (and/or droplets or other partitions) containing macromolecular constituents of biological particles.

Encapsulated biological particles (e.g., cells, B cells, labelled B cells, memory B cells, or plasma cells), e.g., cell beads can provide certain potential advantages of being more storable and more portable than droplet-based partitioned biological particles. Furthermore, in some cases, it can be desirable to allow biological particles (e.g., cells, B cells, labelled B cells, memory B cells, or plasma cells) to incubate for a select period of time before analysis, such as in order to characterize changes in such biological particles over time, either in the presence or absence of different stimuli (e.g., cytokines, antigens, etc.). In such cases, encapsulation or entrainment can allow for longer incubation than partitioning in emulsion droplets, although in some cases, droplet partitioned biological particles can also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles or generation of cell beads from biological particles (e.g., cells, B cells, labelled B cells, memory B cells, or plasma cells) can constitute the partitioning of the biological particles into which other reagents are co-partitioned. Alternatively or in addition, encapsulated biological particles or cell beads can be readily deposited into other partitions (e.g., droplets) as described above.

Microwells

As described herein, one or more processes can be performed in a partition, which can be a well. The well can be a well of a plurality of wells of a substrate, such as a microwell of a microwell array or plate, or the well can be a microwell or microchamber of a device (e.g., microfluidic device) comprising a substrate. The well can be a well of a well array or plate, or the well can be a well or chamber of a device (e.g., fluidic device). Accordingly, the wells or microwells can assume an “open” configuration, in which the wells or microwells are exposed to the environment (e.g., contain an open surface) and are accessible on one planar face of the substrate, or the wells or microwells can assume a “closed” or “sealed” configuration, in which the microwells are not accessible on a planar face of the substrate. In some instances, the wells or microwells can be configured to toggle between “open” and “closed” configurations. For instance, an “open” microwell or set of microwells can be “closed” or “sealed” using a membrane (e.g., semi-permeable membrane), an oil (e.g., fluorinated oil to cover an aqueous solution), or a lid, as described elsewhere herein. The wells or microwells can be initially provided in a “closed” or “sealed” configuration, wherein they are not accessible on a planar surface of the substrate without an external force. For instance, the “closed” or “sealed” configuration can include a substrate such as a sealing film or foil that is puncturable or pierceable by pipette tip(s). Suitable materials for the substrate include, without limitation, polyester, polypropylene, polyethylene, vinyl, and aluminum foil.

In some embodiments, the well can have a volume of less than 1 milliliter (mL). For example, the well can be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well can be configured to hold a volume of about 1000 μL, about 100 μL, about 10 μL, about 1 μL, about 100 nL, about 10 nL, about 1 nL, about 100 pL, about 10 pL, etc. The well can be configured to hold a volume of at least 10 pL, at least 100 pL, at least 1 nL, at least 10 nL, at least 100 nL, at least 1 μL, at least 10 μL, at least 100 μL, at least 1000 μL, or more. The well can be configured to hold a volume in a range of volumes listed herein, for example, from about 5 nL to about 20 nL, from about 1 nL to about 100 nL, from about 500 pL to about 100 μL, etc. The well can be of a plurality of wells that have varying volumes and can be configured to hold a volume appropriate to accommodate any of the partition volumes described herein.

In some instances, a microwell array or plate includes a single variety of microwells. In some instances, a microwell array or plate includes a variety of microwells. For instance, the microwell array or plate can include one or more types of microwells within a single microwell array or plate. The types of microwells can have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate can include any number of different types of microwells. For example, the microwell array or plate can include 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well can have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate includes different types of microwells that are located adjacent to one another within the array or plate. For example, a microwell with one set of dimensions can be located adjacent to and in contact with another microwell with a different set of dimensions. Similarly, microwells of different geometries can be placed adjacent to or in contact with one another. The adjacent microwells can be configured to hold different articles; for example, one microwell can be used to contain a cell, cell bead, or other sample (e.g., cellular components, nucleic acid molecules, nucleic acid barcode molecules, etc.) while the adjacent microwell can be used to contain a microcapsule, droplet, bead, or other reagent. In some cases, the adjacent microwells can be configured to merge the contents held within, e.g., upon application of a stimulus, or spontaneously, upon contact of the articles in each microwell.

As is described elsewhere herein, a plurality of partitions can be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells can include both unoccupied wells (e.g., empty wells) and occupied wells.

A well can include any of the reagents described herein, or combinations thereof. These reagents can include, for example, barcode molecules, enzymes, adapters, and combinations thereof. The reagents can be physically separated from a sample (for example, a cell, cell bead, or cellular components, e.g., proteins, nucleic acid molecules, etc.) that is placed in the well. This physical separation can be accomplished by containing the reagents within, or coupling to, a microcapsule or bead that is placed within a well. The physical separation can also be accomplished by dispensing the reagents in the well and overlaying the reagents with a layer that is, for example, dissolvable, meltable, or permeable prior to introducing the polynucleotide sample into the well. This layer can be, for example, an oil, wax, membrane (e.g., semi-permeable membrane), or the like. The well can be sealed at any point, for example, after addition of the microcapsule or bead, after addition of the reagents, or after addition of either of these components. The sealing of the well can be useful for a variety of purposes, including preventing escape of beads or loaded reagents from the well, permitting select delivery of certain reagents (e.g., via the use of a semi-permeable membrane), for storage of the well prior to or following further processing, etc.

A well can include free reagents and/or reagents encapsulated in, or otherwise coupled to or associated with, microcapsules, beads, or droplets. In some embodiments, any of the reagents described in this disclosure can be encapsulated in, or otherwise coupled to, a microcapsule, droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing can include one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well can be used to perform one or more reactions, including but not limited to: cell lysis, cell fixation, permeabilization, nucleic acid reactions, e.g., nucleic acid extension reactions, amplification, reverse transcription, transposase reactions (e.g., tagmentation), etc.

The wells disclosed herein can be provided as a part of a kit. For example, a kit can include instructions for use, a microwell array or device, and reagents (e.g., beads). The kit can include any useful reagents for performing the processes described herein, e.g., nucleic acid reactions, barcoding of nucleic acid molecules, sample processing (e.g., for cell lysis, fixation, and/or permeabilization).

In some cases, a well includes a microcapsule, bead, or droplet that includes a set of reagents that has a similar attribute, for example, a set of enzymes, a set of minerals, a set of oligonucleotides, a mixture of different barcode molecules, a mixture of identical barcode molecules. In other cases, a microcapsule, bead, or droplet includes a heterogeneous mixture of reagents. In some cases, the heterogeneous mixture of reagents can include all components necessary to perform a reaction. In some cases, such mixture can include all components necessary to perform a reaction, except for 1, 2, 3, 4, 5, or more components necessary to perform a reaction. In some cases, such additional components are contained within, or otherwise coupled to, a different microcapsule, droplet, or bead, or within a solution within a partition (e.g., microwell) of the system.

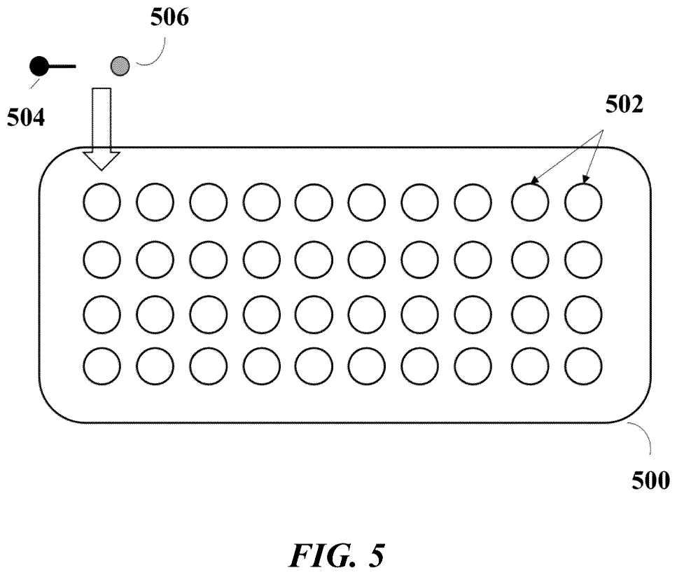

A non-limiting example of a microwell array in accordance with some embodiments of the disclosure is schematically presented in FIG. 5. In this example, the array can be contained within a substrate 500. The substrate 500 includes a plurality of wells 502. The wells 502 can be of any size or shape, and the spacing between the wells, the number of wells per substrate, as well as the density of the wells on the substrate 500 can be modified, depending on the particular application. In one such example application, a sample molecule 506, which can include a cell or cellular components (e.g., nucleic acid molecules) is co-partitioned with a bead 504, which can include a nucleic acid barcode molecule coupled thereto. The wells 502 can be loaded using gravity or other loading technique (e.g., centrifugation, liquid handler, acoustic loading, optoelectronic, etc.). In some instances, at least one of the wells 502 contains a single sample molecule 506 (e.g., cell) and a single bead 504.

Reagents can be loaded into a well either sequentially or concurrently. In some cases, reagents are introduced to the device either before or after a particular operation. In some cases, reagents (which can be provided, in certain instances, in microcapsules, droplets, or beads) are introduced sequentially such that different reactions or operations occur at different steps. The reagents (or microcapsules, droplets, or beads) can also be loaded at operations interspersed with a reaction or operation step. For example, microcapsules (or droplets or beads) including reagents for fragmenting polynucleotides (e.g., restriction enzymes) and/or other enzymes (e.g., transposases, ligases, polymerases, etc.) can be loaded into the well or plurality of wells, followed by loading of microcapsules, droplets, or beads including reagents for attaching nucleic acid barcode molecules to a sample nucleic acid molecule. Reagents can be provided concurrently or sequentially with a sample, e.g., a cell or cellular components (e.g., organelles, proteins, nucleic acid molecules, carbohydrates, lipids, etc.). Accordingly, use of wells can be useful in performing multi-step operations or reactions.

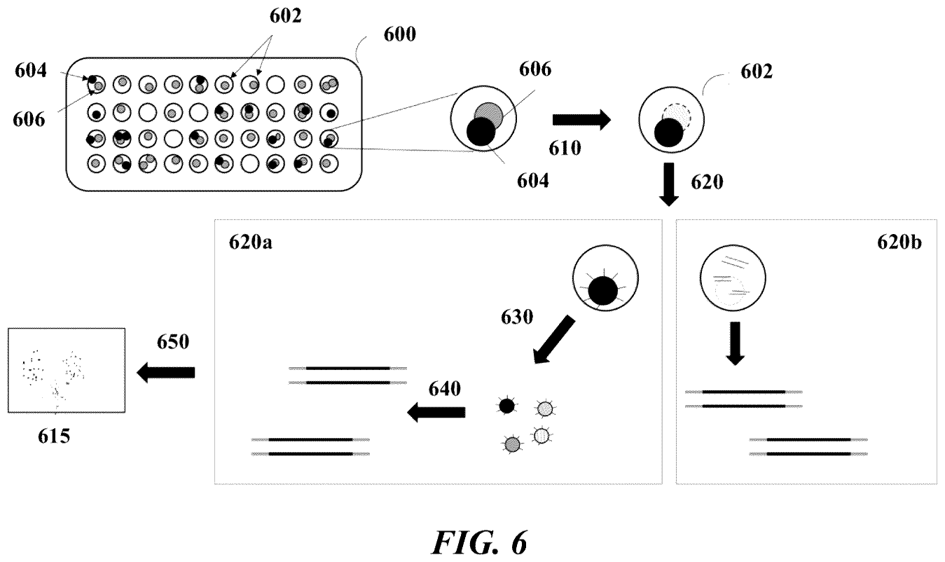

FIG. 6 schematically shows an example workflow for processing nucleic acid molecules within a sample. A substrate 600 including a plurality of microwells 602 can be provided. A sample 606 which can include a cell, cell bead, cellular components or analytes (e.g., proteins and/or nucleic acid molecules) can be co-partitioned, in a plurality of microwells 602, with a plurality of beads 604 including nucleic acid barcode molecules. During a partitioning process, the sample 606 can be processed within the partition. For instance, in the case of live cells, the cell can be subjected to conditions sufficient to lyse the cells and release the analytes contained therein. In process 620, the bead 604 can be further processed. By way of example, processes 620a and 620b schematically illustrate different workflows, depending on the properties of the bead 604.

In 620a, the bead includes nucleic acid barcode molecules that are attached thereto, and sample nucleic acid molecules (e.g., RNA, DNA) can attach, e.g., via hybridization of ligation, to the nucleic acid barcode molecules. Such attachment can occur on the bead. In process 630, the beads 604 from multiple wells 602 can be collected and pooled. Further processing can be performed in process 640. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 650, further characterization, such as sequencing can be performed to generate sequencing reads. The sequencing reads can yield information on individual cells or populations of cells, which can be represented visually or graphically, e.g., in a plot.

As described elsewhere herein, the nucleic acid barcode molecules and other reagents can be contained within a microcapsule, bead, or droplet. These microcapsules, beads, or droplets can be loaded into a partition (e.g., a microwell) before, after, or concurrently with the loading of a cell, such that each cell is contacted with a different microcapsule, bead, or droplet. This technique can be used to attach a unique nucleic acid barcode molecule to nucleic acid molecules obtained from each cell. Alternatively or in addition, the sample nucleic acid molecules can be attached to a support. For example, the partition (e.g., microwell) can include a bead which has coupled thereto a plurality of nucleic acid barcode molecules. The sample nucleic acid molecules, or derivatives thereof, can couple or attach to the nucleic acid barcode molecules attached on the support. The resulting barcoded nucleic acid molecules can then be removed from the partition, and in some instances, pooled and sequenced. In such cases, the nucleic acid barcode sequences can be used to trace the origin of the sample nucleic acid molecule. For example, polynucleotides with identical barcodes can be determined to originate from the same cell or partition, while polynucleotides with different barcodes can be determined to originate from different cells or partitions.

The samples or reagents can be loaded in the wells or microwells using a variety of approaches. For example, the samples (e.g., a cell, cell bead, or cellular component) or reagents (as described herein) can be loaded into the well or microwell using an external force, e.g., gravitational force, electrical force, magnetic force, or using mechanisms to drive the sample or reagents into the well, for example, via pressure-driven flow, centrifugation, optoelectronics, acoustic loading, electrokinetic pumping, vacuum, capillary flow, etc. In certain cases, a fluid handling system can be used to load the samples or reagents into the well. The loading of the samples or reagents can follow a Poissonian distribution or a non-Poissonian distribution, e.g., super Poisson or sub-Poisson. The geometry, spacing between wells, density, and size of the microwells can be modified to accommodate a useful sample or reagent distribution; for example, the size and spacing of the microwells can be adjusted such that the sample or reagents can be distributed in a super-Poissonian fashion.

In one non-limiting example, the microwell array or plate includes pairs of microwells, in which each pair of microwells is configured to hold a droplet (e.g., including a single cell) and a single bead (such as those described herein, which can, in some instances, also be encapsulated in a droplet). The droplet and the bead (or droplet containing the bead) can be loaded simultaneously or sequentially, and the droplet and the bead can be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets including different reagents and/or samples, which are merged upon contact or upon application of a stimulus. In such instances, the droplet of one microwell of the pair can include reagents that can react with an agent in the droplet of the other microwell of the pair. For example, one droplet can include reagents that are configured to release the nucleic acid barcode molecules of a bead contained in another droplet, located in the adjacent microwell. Upon merging of the droplets, the nucleic acid barcode molecules can be released from the bead into the partition (e.g., the microwell or microwell pair that are in contact), and further processing can be performed (e.g., barcoding, nucleic acid reactions, etc.). In cases where intact or live cells are loaded in the microwells, one of the droplets can include lysis reagents for lysing the cell upon droplet merging.

In some embodiments, a droplet or microcapsule (e.g., bead) can be partitioned into a well. The droplets can be selected or subjected to pre-processing prior to loading into a well. For instance, the droplets can include cells, and only certain droplets, such as those containing a single cell (or at least one cell), can be selected for use in loading of the wells. Such a pre-selection process can be useful in efficient loading of single cells, such as to obtain a non-Poissonian distribution, or to pre-filter cells for a selected characteristic prior to further partitioning in the wells. Additionally, the technique can be useful in obtaining or preventing cell doublet or multiplet formation prior to or during loading of the microwell.

In some embodiments, the wells can include nucleic acid barcode molecules attached thereto. The nucleic acid barcode molecules can be attached to a surface of the well (e.g., a wall of the well). The nucleic acid barcode molecule (e.g., a partition barcode sequence) of one well can differ from the nucleic acid barcode molecule of another well, which can permit identification of the contents contained with a single partition or well. In some embodiments, the nucleic acid barcode molecule can include a spatial barcode sequence that can identify a spatial coordinate of a well, such as within the well array or well plate. In some embodiments, the nucleic acid barcode molecule can include a unique molecular identifier for individual molecule identification. In some instances, the nucleic acid barcode molecules can be configured to attach to or capture a nucleic acid molecule from or within a sample or cell distributed in the well. For example, the nucleic acid barcode molecules can include a capture sequence that can be used to capture or hybridize to a nucleic acid molecule (e.g., RNA, DNA) from or within the sample. In some embodiments, the nucleic acid barcode molecules can be releasable from the microwell. For example, the nucleic acid barcode molecules can include a chemical cross-linker which can be cleaved upon application of a stimulus (e.g., photo-, magnetic, chemical, biological, stimulus). The released nucleic acid barcode molecules, which can be hybridized or configured to hybridize to a sample nucleic acid molecule, can be collected and pooled for further processing, which can include nucleic acid processing (e.g., amplification, extension, reverse transcription, etc.) and/or characterization (e.g., sequencing). In such cases, the unique partition barcode sequences can be used to identify the cell or partition from which a nucleic acid molecule originated.

Characterization of samples within a well can be performed. Such characterization can include, in non-limiting examples, imaging of the sample (e.g., cell, cell bead, or cellular components) or derivatives thereof. Characterization techniques such as microscopy or imaging can be useful in measuring sample profiles in fixed spatial locations. For example, when cells are partitioned, optionally with beads, imaging of each microwell and the contents contained therein can provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging can be used to characterize live cells in the wells, including, but not limited to: dynamic live-cell tracking, cell-cell interactions (when two or more cells are co-partitioned), cell proliferation, etc. Alternatively or in addition to, imaging can be used to characterize a quantity of amplification products in the well.

In operation, a well can be loaded with a sample and reagents, simultaneously or sequentially. When cells or cell beads are loaded, the well can be subjected to washing, e.g., to remove excess cells from the well, microwell array, or plate. Similarly, washing can be performed to remove excess beads or other reagents from the well, microwell array, or plate. In the instances where live cells are used, the cells can be lysed in the individual partitions to release the intracellular components or cellular analytes. Alternatively, the cells can be fixed or permeabilized in the individual partitions. The intracellular components or cellular analytes can couple to a support, e.g., on a surface of the microwell, on a solid support (e.g., bead), or they can be collected for further downstream processing. For example, after cell lysis, the intracellular components or cellular analytes can be transferred to individual droplets or other partitions for barcoding. Alternatively, or in addition, the intracellular components or cellular analytes (e.g., nucleic acid molecules) can couple to a bead including a nucleic acid barcode molecule; subsequently, the bead can be collected and further processed, e.g., subjected to nucleic acid reaction such as reverse transcription, amplification, or extension, and the nucleic acid molecules thereon can be further characterized, e.g., via sequencing. Alternatively, or in addition, the intracellular components or cellular analytes can be barcoded in the well (e.g., using a bead including nucleic acid barcode molecules that are releasable or on a surface of the microwell including nucleic acid barcode molecules). The barcoded nucleic acid molecules or analytes can be further processed in the well, or the barcoded nucleic acid molecules or analytes can be collected from the individual partitions and subjected to further processing outside the partition. Further processing can include nucleic acid processing (e.g., performing an amplification, extension) or characterization (e.g., fluorescence monitoring of amplified molecules, sequencing). At any convenient, suitable, and/or useful step, the well (or microwell array or plate) can be sealed (e.g., using an oil, membrane, wax, etc.), which enables storage of the assay or selective introduction of additional reagents.

Beads

In some embodiments of the disclosure, a partition can include one or more unique identifiers, such as barcodes (e.g., a plurality of nucleic acid barcode molecules (e.g., barcode nucleic acid molecules) which can be, for example, a plurality of partition barcode sequences). Barcodes can be previously, subsequently or concurrently delivered to the partitions that hold the compartmentalized or partitioned biological particle (e.g., cell, B cell, labelled B cell, memory B cell, or plasma cell). For example, barcodes can be injected into droplets previous to, subsequent to, or concurrently with droplet generation. In some embodiments, the delivery of the barcodes to a particular partition allows for the later attribution of the characteristics of the individual biological particle (e.g., cell, B cell, labelled B cell, memory B cell, or plasma cell) to the particular partition. Barcodes can be delivered, for example on a nucleic acid molecule, e.g., nucleic acid barcode molecule (e.g., a barcoded oligonucleotide, barcoded nucleic acid molecules), to a partition via any suitable mechanism. In some embodiments, nucleic acid barcode molecules (e.g., a barcoded oligonucleotide, barcoded nucleic acid molecules) can be delivered to a partition via a microcapsule. A microcapsule, in some instances, can include a bead. Beads are described in further detail below.

In some embodiments, barcoded nucleic acid molecules (e.g., nucleic acid barcode molecules) can be initially associated with the microcapsule (e.g., bead) and then released from the microcapsule. In some embodiments, release of the barcoded nucleic acid molecules (e.g., nucleic acid barcode molecules) can be passive (e.g., by diffusion out of the microcapsule, e.g., bead). In addition or alternatively, release from the microcapsule, e.g., bead can be upon application of a stimulus which allows the barcoded nucleic acid nucleic acid molecules (e.g., nucleic acid barcode molecules) to dissociate or to be released from the microcapsule, e.g., bead. Such stimulus can disrupt the microcapsule (e.g., bead), an interaction that couples the barcoded nucleic acid molecules to or within the microcapsule (e.g., bead), or both. Such stimulus can include, for example, a thermal stimulus, photo-stimulus, chemical stimulus (e.g., change in pH or use of a reducing agent), a mechanical stimulus, a radiation stimulus; a biological stimulus (e.g., enzyme), or any combination thereof. Methods and systems for partitioning barcode carrying beads into droplets are provided in US. Patent Publication Nos. 2019/0367997 and 2019/0064173, and International Application Nos. PCT/US20/17785 and PCT/US20/020486.

Beneficially, a discrete droplet partitioning a biological particle and a barcode carrying bead can effectively allow the attribution of the barcode to macromolecular constituents of the biological particle within the partition. The contents of a partition can remain discrete from the contents of other partitions.

In operation, the barcoded oligonucleotides can be released (e.g., in a partition), as described elsewhere herein. Alternatively, the nucleic acid molecules (e.g., nucleic acid barcode molecules) bound to the bead (e.g., gel bead) can be used to hybridize and capture analytes (e.g., one or more types of analytes) on the solid phase of the bead.

In some examples, beads, biological particles (e.g., cells, B cells, labelled B cells, memory B cells, or plasma cells) and droplets can flow along channels (e.g., the channels of a microfluidic device), in some cases at substantially regular flow profiles (e.g., at regular flow rates). Such regular flow profiles can permit a droplet to include a single bead and a single biological particle. Such regular flow profiles can permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that can be used to provide such regular flow profiles are provided in, for example, U.S. Patent Publication No. 2015/0292988, which is entirely incorporated herein by reference.

A bead can be porous, non-porous, solid, semi-solid, semi-fluidic, fluidic, and/or a combination thereof. In some instances, a bead can be dissolvable, disruptable, and/or degradable. In some cases, a bead cannot be degradable. In some cases, the bead can be a gel bead. A gel bead can be a hydrogel bead. A gel bead can be formed from molecular precursors, such as a polymeric or monomeric species. A semi-solid bead can be a liposomal bead. Solid beads can include metals including iron oxide, gold, and silver. In some cases, the bead can be a silica bead. In some cases, the bead can be rigid. In other cases, the bead can be flexible and/or compressible.

A bead can be of any suitable shape. Examples of bead shapes include, but are not limited to, spherical, non-spherical, oval, oblong, amorphous, circular, cylindrical, and variations thereof.

Beads can be of uniform size or heterogeneous size. In some cases, the diameter of a bead can be at least about 10 nanometers (nm), 100 nm, 500 nm, 1 micrometer (μm), 5 μm, 20 μm, 30 μm, 40 μm, 50 μm, 60 μm, 70 μm, 80 μm, 90 μm, 100 μm, 250 μm, 500 μm, 1 mm, or greater. In some cases, a bead can have a diameter of less than about 10 nm, 100 nm, 500 nm, 1 μm, 10 μm, 20 μm, 30 μm, 40 μm, 50 μm, 60 μm, 70 μm, 80 μm, 90 μm, 100μm, 250 μm, 500 μm, 1 mm, or less. In some cases, a bead can have a diameter in the range of about 40-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In some embodiments, the beads described herein can have size distributions that have a coefficient of variation in their cross-sectional dimensions of less than 50%, less than 40%, less than 30%, less than 20%, and in some cases less than 15%, less than 10%, less than 5%, or less.

A bead can include natural and/or synthetic materials. For example, a bead can include a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads can also be formed from materials other than polymers, including lipids, micelles, ceramics, glass-ceramics, material composites, metals, other inorganic materials, and others.

In some embodiments, the bead can contain molecular precursors (e.g., monomers or polymers), which can form a polymer network via polymerization of the molecular precursors. In some cases, a precursor can be an already polymerized species capable of undergoing further polymerization via, for example, a chemical cross-linkage. In some embodiments, a precursor can include one or more of an acrylamide or a methacrylamide monomer, oligomer, or polymer. In some cases, the bead can include prepolymers, which are oligomers capable of further polymerization. For example, polyurethane beads can be prepared using prepolymers. In some embodiments, the bead can contain individual polymers that can be further polymerized together. In some cases, beads can be generated via polymerization of different precursors, such that they include mixed polymers, co-polymers, and/or block co-polymers. In some embodiments, the bead can include covalent or ionic bonds between polymeric precursors (e.g., monomers, oligomers, linear polymers), nucleic acid molecules (e.g., nucleic acid barcode molecules or barcoded oligonucleotides), primers, and other entities. In some embodiments, the covalent bonds can be carbon-carbon bonds, thioether bonds, or carbon-heteroatom bonds.

Cross-linking can be permanent or reversible, depending upon the particular cross-linker used. Reversible cross-linking can allow for the polymer to linearize or dissociate under appropriate conditions. In some embodiments, reversible cross-linking can also allow for reversible attachment of a material bound to the surface of a bead. In some embodiments, a cross-linker can form disulfide linkages. In some embodiments, the chemical cross-linker forming disulfide linkages can be cystamine or a modified cystamine.

In some embodiments, disulfide linkages can be formed between molecular precursor units (e.g., monomers, oligomers, or linear polymers) or precursors incorporated into a bead and nucleic acid molecules (e.g., oligonucleotides, nucleic acid barcode molecules). Cystamine (including modified cystamines), for example, is an organic agent including a disulfide bond that can be used as a crosslinker agent between individual monomeric or polymeric precursors of a bead. Polyacrylamide can be polymerized in the presence of cystamine or a species including cystamine (e.g., a modified cystamine) to generate polyacrylamide gel beads including disulfide linkages (e.g., chemically degradable beads including chemically-reducible cross-linkers). The disulfide linkages can permit the bead to be degraded (or dissolved) upon exposure of the bead to a reducing agent.

In some embodiments, chitosan, a linear polysaccharide polymer, can be crosslinked with glutaraldehyde via hydrophilic chains to form a bead. Crosslinking of chitosan polymers can be achieved by chemical reactions that are initiated by heat, pressure, change in pH, and/or radiation.

In some embodiments, a bead can include an acrydite moiety, which in certain aspects can be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties can be modified to form chemical bonds with a species to be attached, such as a nucleic acid molecule (e.g., barcode sequence, barcoded nucleic acid molecule, nucleic acid barcode molecule, barcoded oligonucleotide, primer, or other oligonucleotide). Acrydite moieties can be modified with thiol groups capable of forming a disulfide bond or can be modified with groups already including a disulfide bond. The thiol or disulfide (via disulfide exchange) can be used as an anchor point for a species to be attached or another part of the acrydite moiety can be used for attachment. In some cases, attachment can be reversible, such that when the disulfide bond is broken (e.g., in the presence of a reducing agent), the attached species is released from the bead. In other cases, an acrydite moiety can include a reactive hydroxyl group that can be used for attachment.

Functionalization of beads for attachment of nucleic acid molecules (e.g., oligonucleotides, nucleic acid barcode molecules) can be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead can include acrydite moieties, such that when a bead is generated, the bead also includes acrydite moieties. The acrydite moieties can be attached to a nucleic acid molecule (e.g., oligonucleotide, nucleic acid barcode molecule), which can include a priming sequence (e.g., a primer for amplifying target nucleic acids, random primer, primer sequence for messenger RNA) and/or one or more barcode sequences. The one or more barcode sequences can include sequences that are the same for all nucleic acid molecules (e.g., nucleic acid barcode molecules) coupled to a given bead and/or sequences that are different across all nucleic acid molecules (e.g., nucleic acid barcode molecules) coupled to the given bead. The nucleic acid barcode molecule can be incorporated into the bead.

In some embodiments, the nucleic acid molecule (e.g., nucleic acid barcode molecule) can include a functional sequence, for example, for attachment to a sequencing flow cell, such as, for example, a P5 sequence for Illumina® sequencing. In some cases, the nucleic acid molecule (e.g., nucleic acid barcode molecule) or derivative thereof (e.g., oligonucleotide or polynucleotide generated from the nucleic acid molecule) can include another functional sequence, such as, for example, a P7 sequence for attachment to a sequencing flow cell for Illumina sequencing. In some cases, the nucleic acid molecule can include a barcode sequence. In some cases, the primer can further include a unique molecular identifier (UMI). In some cases, the primer can include an R1 primer sequence for Illumina sequencing. In some cases, the primer can include an R2 primer sequence for Illumina sequencing. Examples of such nucleic acid molecules (e.g., oligonucleotides, polynucleotides, etc.) and uses thereof, as can be used with compositions, devices, methods and systems of the present disclosure, are provided in U.S. Patent Pub. Nos. 2014/0378345 and 2015/0376609.

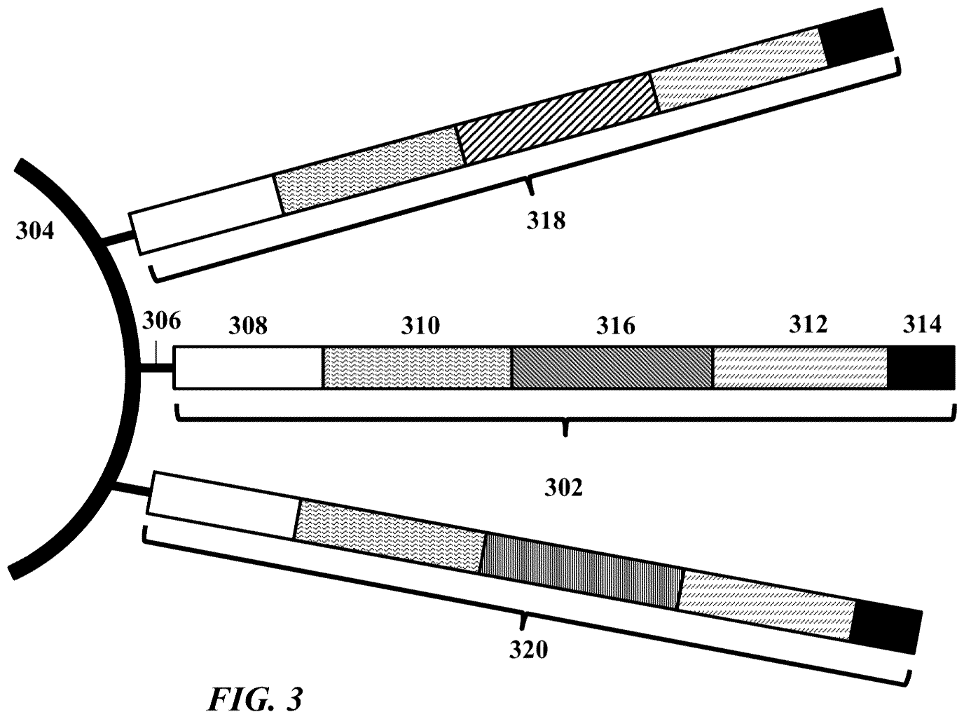

FIG. 3 illustrates an example of a barcode carrying bead. A nucleic acid molecule 302, such as an oligonucleotide, can be coupled to a bead 304 by a releasable linkage 306, such as, for example, a disulfide linker. The same bead 304 can be coupled (e.g., via releasable linkage) to one or more other nucleic acid molecules (e.g., nucleic acid barcode molecules) 318, 320. The nucleic acid molecule 302 can be or include a barcode. As noted elsewhere herein, the structure of the barcode can include a number of sequence elements. The nucleic acid molecule (nucleic acid barcode molecule) 302 can include a functional sequence 308 that can be used in subsequent processing. For example, the functional sequence 308 can include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 302 can include a barcode sequence 310 for use in barcoding the sample (e.g., DNA, RNA, protein, etc.). In some cases, the barcode sequence 310 can be bead-specific such that the barcode sequence 310 is common to all nucleic acid molecules (e.g., including nucleic acid barcode molecule 302) coupled to the same bead 304. Alternatively or in addition, the barcode sequence 310 can be partition-specific such that the barcode sequence 310 is common to all nucleic acid molecules coupled to one or more beads that are partitioned into the same partition. The nucleic acid molecule (nucleic acid barcode molecule) 302 can include a specific priming sequence 312, such as an mRNA specific priming sequence (e.g., poly-T sequence), a targeted priming sequence, and/or a random priming sequence. The nucleic acid molecule 302 can include an anchoring sequence 314 to ensure that the specific priming sequence 312 hybridizes at the sequence end (e.g., of the mRNA). For example, the anchoring sequence 314 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule (e.g., nucleic acid barcode molecule) 302 can include a unique molecular identifying sequence 316 (e.g., unique molecular identifier (UMI)). In some cases, the unique molecular identifying sequence 316 can include from about 5 to about 8 nucleotides. Alternatively, the unique molecular identifying sequence 316 can compress less than about 5 or more than about 8 nucleotides. The unique molecular identifying sequence 316 can be a unique sequence that varies across individual nucleic acid molecules (e.g., 302, 318, 320, etc.) coupled to a single bead (e.g., bead 304). In some cases, the unique molecular identifying sequence 316 can be a random sequence (e.g., such as a random N-mer sequence). For example, the UMI can provide a unique identifier of the starting mRNA molecule that was captured, in order to allow quantitation of the number of original expressed RNA. As will be appreciated, although FIG. 3 shows three nucleic acid molecules 302, 318, 320 coupled to the surface of the bead 304, an individual bead can be coupled to any number of individual nucleic acid molecules, for example, from one to tens to hundreds of thousands, millions, or even billion of individual nucleic acid barcode molecules. The respective barcodes for the individual nucleic acid molecules can include both common sequence segments or relatively common sequence segments (e.g., 308, 310, 312, etc.) and variable or unique sequence segments (e.g., 316) between different individual nucleic acid molecules coupled to the same bead.

In operation, a biological particle (e.g., cell, DNA, RNA, etc.) can be co-partitioned along with a barcode bearing bead 304. The barcoded nucleic acid molecules (e.g., nucleic acid barcode molecules) 302, 318, 320 can be released from the bead 304 in the partition. By way of example, in the context of analyzing sample RNA, the poly-T segment (e.g., 312) of one of the released nucleic acid molecules (e.g., nucleic acid barcode molecules) (e.g., 302) can hybridize to the poly-A tail of an mRNA molecule. Reverse transcription can result in a cDNA transcript of the mRNA, but which transcript includes each of the sequence segments 308, 310, 316 of the nucleic acid molecule 302. Because the nucleic acid molecule 302 includes an anchoring sequence 314, it will more likely hybridize to and prime reverse transcription at the sequence end of the poly-A tail of the mRNA. Within any given partition, all of the cDNA transcripts of the individual mRNA molecules can include a common barcode sequence segment 310. However, the transcripts made from the different mRNA molecules within a given partition can vary at the unique molecular identifying sequence 312 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences can also be used in priming the reverse transcription reaction. Likewise, although described as releasing the barcoded oligonucleotides into the partition, in some cases, the nucleic acid molecules bound to the bead (e.g., gel bead) can be used to hybridize and capture the mRNA on the solid phase of the bead, for example, in order to facilitate the separation of the RNA from other cell contents. In such cases, further processing can be performed, in the partitions or outside the partitions (e.g., in bulk). For instance, the RNA molecules on the beads can be subjected to reverse transcription or other nucleic acid processing, additional adapter sequences can be added to the barcoded nucleic acid molecules, or other nucleic acid reactions (e.g., amplification, nucleic acid extension) can be performed. The beads or products thereof (e.g., barcoded nucleic acid molecules) can be collected from the partitions, and/or pooled together and subsequently subjected to clean up and further characterization (e.g., sequencing).

The operations described herein can be performed at any useful or convenient step. For instance, the beads including nucleic acid barcode molecules can be introduced into a partition (e.g., well or droplet) prior to, during, or following introduction of a sample into the partition. The nucleic acid molecules of a sample can be subjected to barcoding, which can occur on the bead (in cases where the nucleic acid molecules remain coupled to the bead) or following release of the nucleic acid barcode molecules into the partition. In cases where the nucleic acid molecules from the sample remain attached to the bead, the beads from various partitions can be collected, pooled, and subjected to further processing (e.g., reverse transcription, adapter attachment, amplification, clean up, and/or sequencing). In other instances, the processing can occur in the partition. For example, conditions sufficient for barcoding, adapter attachment, reverse transcription, or other nucleic acid processing operations can be provided in the partition and performed prior to clean up and sequencing.

In some instances, a bead can include a capture sequence or binding sequence configured to bind to a corresponding capture sequence or binding sequence. In some instances, a bead can include a plurality of different capture sequences or binding sequences configured to bind to different respective corresponding capture sequences or binding sequences. For example, a bead can include a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead can include any number of different capture sequences. In some instances, a bead can include at least 2, 3, 4, 5, 6, 7, 8, 9, 10 or more different capture sequences or binding sequences configured to bind to different respective capture sequences or binding sequences, respectively. Alternatively or in addition, a bead can include at most about 10, 9, 8, 7, 6, 5, 4, 3, or 2 different capture sequences or binding sequences configured to bind to different respective capture sequences or binding sequences. In some instances, the different capture sequences or binding sequences can be configured to facilitate analysis of a same type of analyte. In some instances, the different capture sequences or binding sequences can be configured to facilitate analysis of different types of analytes (with the same bead). The capture sequence can be designed to attach to a corresponding capture sequence. Beneficially, such corresponding capture sequence can be introduced to, or otherwise induced in, a biological particle (e.g., cell, cell bead, etc.) for performing different assays in various formats (e.g., barcoded antibodies including the corresponding capture sequence, barcoded MHC dextramers including the corresponding capture sequence, barcoded guide RNA molecules including the corresponding capture sequence, etc.), such that the corresponding capture sequence can later interact with the capture sequence associated with the bead. In some instances, a capture sequence coupled to a bead (or other support) can be configured to attach to a linker molecule, such as a splint molecule, wherein the linker molecule is configured to couple the bead (or other support) to other molecules through the linker molecule, such as to one or more analytes or one or more other linker molecules.

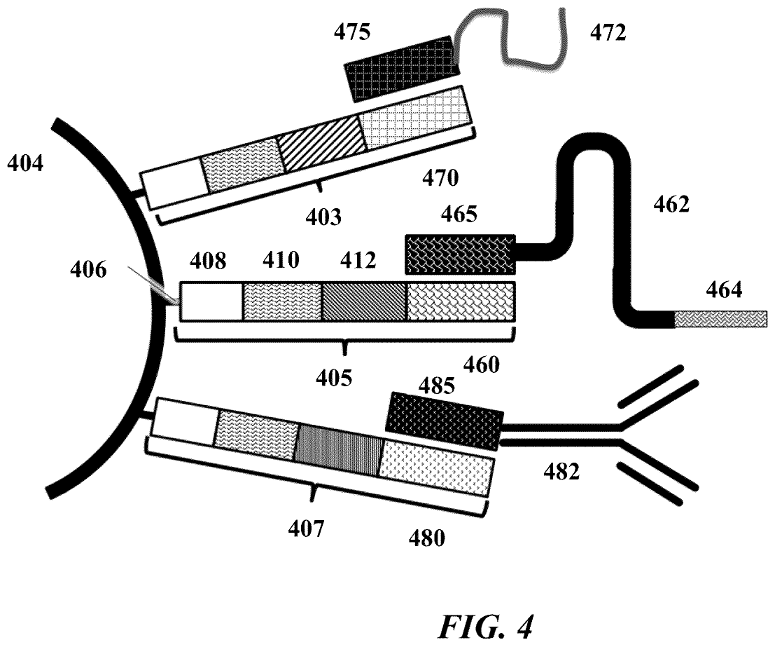

FIG. 4 illustrates a non-limiting example of a barcode carrying bead in accordance with some embodiments of the disclosure. A nucleic acid molecule (e.g., nucleic acid barcode molecule) 405, such as an oligonucleotide, can be coupled to a bead 404 by a releasable linkage 406, such as, for example, a disulfide linker. The nucleic acid molecule 405 can include a first capture sequence 460. The same bead 404 can be coupled, e.g., via releasable linkage, to one or more other nucleic acid molecules 403, 407 including other capture sequences. The nucleic acid molecule 405 can be or include a barcode, e.g., barcode sequence. As described elsewhere herein, the structure of the barcode (e.g., barcode sequence) can include a number of sequence elements, such as a functional sequence 408 (e.g., flow cell attachment sequence, sequencing primer sequence, etc.), a barcode sequence 410 (e.g., bead-specific sequence common to bead, partition-specific sequence common to partition, etc.), and a unique molecular identifier 412 (e.g., unique sequence within different molecules attached to the bead), or partial sequences thereof. The capture sequence 460 can be configured to attach to a corresponding capture sequence 465 (e.g., capture handle). In some instances, the corresponding capture sequence 465 can be coupled to another molecule that can be an analyte or an intermediary carrier. For example, as illustrated in FIG. 4, the corresponding capture sequence 465 is coupled to a guide RNA molecule 462 including a target sequence 464, wherein the target sequence 464 is configured to attach to the analyte. Another oligonucleotide molecule 407 attached to the bead 404 includes a second capture sequence 480 which is configured to attach to a second corresponding capture sequence (e.g., capture handle) 485. As illustrated in FIG. 4, the second corresponding capture sequence 485 is coupled to an antibody 482. In some cases, the antibody 482 can have binding specificity to an analyte (e.g., surface protein). Alternatively, the antibody 482 cannot have binding specificity. Another oligonucleotide molecule 403 attached to the bead 404 includes a third capture sequence 470 which is configured to attach to a second corresponding capture sequence 475. As illustrated in FIG. 4, the third corresponding capture sequence (e.g., capture handle) 475 is coupled to a molecule 472. The molecule 472 may or may not be configured to target an analyte. The other oligonucleotide molecules 403, 407 can include the other sequences (e.g., functional sequence, barcode sequence, UMI, etc.) described with respect to oligonucleotide molecule 405. While a single oligonucleotide molecule including each capture sequence is illustrated in FIG. 4, it will be appreciated that, for each capture sequence, the bead can include a set of one or more oligonucleotide molecules each including the capture sequence. For example, the bead can include any number of sets of one or more different capture sequences. Alternatively or in addition, the bead 404 can include other capture sequences. Alternatively or in addition, the bead 404 can include fewer types of capture sequences (e.g., two capture sequences). Alternatively or in addition, the bead 404 can include oligonucleotide molecule(s) including a priming sequence, such as a specific priming sequence such as an mRNA specific priming sequence (e.g., poly-T sequence), a targeted priming sequence, and/or a random priming sequence, for example, to facilitate an assay for gene expression.

The generation of a barcoded sequence, see, e.g., FIG. 3, is described herein.

In some embodiments, precursors including a functional group that is reactive or capable of being activated such that it becomes reactive can be polymerized with other precursors to generate gel beads including the activated or activatable functional group. The functional group can then be used to attach additional species (e.g., disulfide linkers, primers, other oligonucleotides, etc.) to the gel beads. For example, some precursors including a carboxylic acid (COOH) group can co-polymerize with other precursors to form a gel bead that also includes a COOH functional group. In some cases, acrylic acid (a species including free COOH groups), acrylamide, and bis(acryloyl)cystamine can be co-polymerized together to generate a gel bead including free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species including an amine functional group where the carboxylic acid groups are activated to be reactive with an amine functional group) including a moiety to be linked to the bead.

Beads including disulfide linkages in their polymeric network can be functionalized with additional species via reduction of some of the disulfide linkages to free thiols. The disulfide linkages can be reduced via, for example, the action of a reducing agent (e.g., DTT, TCEP, etc.) to generate free thiol groups, without dissolution of the bead. Free thiols of the beads can then react with free thiols of a species or a species including another disulfide bond (e.g., via thiol-disulfide exchange) such that the species can be linked to the beads (e.g., via a generated disulfide bond). In some cases, free thiols of the beads can react with any other suitable group. For example, free thiols of the beads can react with species including an acrydite moiety. The free thiol groups of the beads can react with the acrydite via Michael addition chemistry, such that the species including the acrydite is linked to the bead. In some cases, uncontrolled reactions can be prevented by inclusion of a thiol capping agent such as N-ethylmaleimide or iodoacetate.

Activation of disulfide linkages within a bead can be controlled such that only a small number of disulfide linkages are activated. Control can be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent can be used for reduction. Controlling the number of disulfide linkages that are reduced to free thiols can be useful in ensuring bead structural integrity during functionalization. In some cases, optically-active agents, such as fluorescent dyes can be coupled to beads via free thiol groups of the beads and used to quantify the number of free thiols present in a bead and/or track a bead.

In some embodiments, addition of moieties to a gel bead after gel bead formation can be advantageous. For example, addition of an oligonucleotide (e.g., barcoded oligonucleotide, such as a barcoded nucleic acid molecule, e.g., nucleic acid barcode molecule) after gel bead formation can avoid loss of the species during chain transfer termination that can occur during polymerization. Moreover, smaller precursors (e.g., monomers or cross linkers that do not include side chain groups and linked moieties) can be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel can possess an upper critical solution temperature (UCST) that can permit temperature driven swelling and collapse of a bead. Such functionality can aid in oligonucleotide (e.g., a primer) infiltration into the bead during subsequent functionalization of the bead with the oligonucleotide. Post-production functionalization can also be useful in controlling loading ratios of species in beads, such that, for example, the variability in loading ratio is minimized. Species loading can also be performed in a batch process such that a plurality of beads can be functionalized with the species in a single batch.

A bead injected or otherwise introduced into a partition can include releasably, cleavably, or reversibly attached barcodes (e.g., partition barcode sequences). A bead injected or otherwise introduced into a partition can include activatable barcodes. A bead injected or otherwise introduced into a partition can be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage can be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules, e.g., nucleic acid barcode molecules (e.g., barcoded oligonucleotides), the beads can be degradable, disruptable, or dissolvable spontaneously or upon exposure to one or more stimuli (e.g., temperature changes, pH changes, exposure to particular chemical species or phase, exposure to light, reducing agent, etc.). In some cases, a bead can be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead can be thermally degradable such that when the bead is exposed to an appropriate change in temperature (e.g., heat), the bead degrades. Degradation or dissolution of a bead bound to a species (e.g., a nucleic acid molecule, e.g., nucleic acid barcode molecule or barcoded oligonucleotide) can result in release of the species from the bead.

As will be appreciated from the above disclosure, the degradation of a bead can refer to the disassociation of a bound (e.g., capture agent configured to couple to a secreted antibody or antigen binding fragment thereof) or entrained species (e.g., labelled B cell or plasma cell, or secreted antibody or antigen binding fragment thereof) from a bead, both with and without structurally degrading the physical bead itself. For example, the degradation of the bead can involve cleavage of a cleavable linkage via one or more species and/or methods described elsewhere herein. In another example, entrained species can be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

A degradable bead can be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules, nucleic acid barcode molecules) can interact with other reagents contained in the partition. For example, a polyacrylamide bead including cystamine and linked, via a disulfide bond, to a barcode sequence, can be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet including a bead-bound barcode sequence in basic solution can also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing nucleic acid molecule (e.g., nucleic acid barcode molecule, oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads can be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads can be accomplished by various swelling methods. The de-swelling of the beads can be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads can be accomplished by various de-swelling methods. Transferring the beads can cause pores in the bead to shrink. The shrinking can then hinder reagents within the beads from diffusing out of the interiors of the beads. The hindrance can be due to steric interactions between the reagents and the interiors of the beads. The transfer can be accomplished microfluidically. For instance, the transfer can be achieved by moving the beads from one co-flowing solvent stream to a different co-flowing solvent stream. The swellability and/or pore size of the beads can be adjusted by changing the polymer composition of the bead.

In some cases, an acrydite moiety linked to a precursor, another species linked to a precursor, or a precursor itself can include a labile bond, such as chemically, thermally, or photo-sensitive bond e.g., disulfide bond, UV sensitive bond, or the like. Once acrydite moieties or other moieties including a labile bond are incorporated into a bead, the bead can also include the labile bond. The labile bond can be, for example, useful in reversibly linking (e.g., covalently linking) species (e.g., barcodes, primers, etc.) to a bead. In some cases, a thermally labile bond can include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead.

The addition of multiple types of labile bonds to a gel bead can result in the generation of a bead capable of responding to varied stimuli. Each type of labile bond can be sensitive to an associated stimulus (e.g., chemical stimulus, light, temperature, enzymatic, etc.) such that release of species attached to a bead via each labile bond can be controlled by the application of the appropriate stimulus. Such functionality can be useful in controlled release of species from a gel bead. In some cases, another species including a labile bond can be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that can be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond can be cleavable via other nucleic acid molecule targeting enzymes, such as restriction enzymes (e.g., restriction endonucleases), as described further below.

Species can be encapsulated in beads (e.g., capture agent) during bead generation (e.g., during polymerization of precursors). Such species may or may not participate in polymerization. Such species can be entered into polymerization reaction mixtures such that generated beads include the species upon bead formation. In some cases, such species can be added to the gel beads after formation. Such species can include, for example, nucleic acid molecules (e.g., oligonucleotides, e.g., nucleic acid barcode molecules), reagents for a nucleic acid amplification reaction (e.g., primers, polymerases, dNTPs, co-factors (e.g., ionic co-factors, buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species can include one or more enzymes described herein, including without limitation, polymerase, reverse transcriptase, restriction enzymes (e.g., endonuclease), transposase, ligase, proteinase K, DNAse, etc. Such species can include one or more reagents described elsewhere herein (e.g., lysis agents, inhibitors, inactivating agents, chelating agents, stimulus). Trapping of such species can be controlled by the polymer network density generated during polymerization of precursors, control of ionic charge within the gel bead (e.g., via ionic species linked to polymerized species), or by the release of other species. Encapsulated species can be released from a bead upon bead degradation and/or by application of a stimulus capable of releasing the species from the bead. Alternatively or in addition, species can be partitioned in a partition (e.g., droplet) during or subsequent to partition formation. Such species can include, without limitation, the abovementioned species that can also be encapsulated in a bead.

A degradable bead can include one or more species with a labile bond such that, when the bead/species is exposed to the appropriate stimuli, the bond is broken and the bead degrades. The labile bond can be a chemical bond (e.g., covalent bond, ionic bond) or can be another type of physical interaction (e.g., van der Waals interactions, dipole-dipole interactions, etc.). In some cases, a crosslinker used to generate a bead can include a labile bond. Upon exposure to the appropriate conditions, the labile bond can be broken and the bead degraded. For example, upon exposure of a polyacrylamide gel bead including cystamine crosslinkers to a reducing agent, the disulfide bonds of the cystamine can be broken and the bead degraded.

A degradable bead can be useful in more quickly releasing an attached species (e.g., a nucleic acid molecule, a nucleic acid barcode molecule, a barcode sequence, a primer, etc.) from the bead when the appropriate stimulus is applied to the bead as compared to a bead that does not degrade. For example, for a species bound to an inner surface of a porous bead or in the case of an encapsulated species, the species can have greater mobility and accessibility to other species in solution upon degradation of the bead. In some cases, a species can also be attached to a degradable bead via a degradable linker (e.g., disulfide linker). The degradable linker can respond to the same stimuli as the degradable bead or the two degradable species can respond to different stimuli. For example, a barcode sequence can be attached, via a disulfide bond, to a polyacrylamide bead including cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation can refer to the disassociation of a bound or entrained species from a bead, both with and without structurally degrading the physical bead itself. For example, entrained species can be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

Where degradable beads are provided, it can be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads include reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it can be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that can be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers can be used to trigger the degradation of beads. Examples of these chemical changes can include, but are not limited to pH-mediated changes to the integrity of a component within the bead, degradation of a component of a bead via cleavage of cross-linked bonds, and depolymerization of a component of a bead.

In some embodiments, a bead can be formed from materials that include degradable chemical crosslinkers, such as BAC or cystamine. Degradation of such degradable crosslinkers can be accomplished through a number of mechanisms. In some examples, a bead can be contacted with a chemical degrading agent that can induce oxidation, reduction or other chemical changes. For example, a chemical degrading agent can be a reducing agent, such as dithiothreitol (DTT). Additional examples of reducing agents can include β-mercaptoethanol, (2S)-2-amino-1,4-dimercaptobutane (dithiobutylamine or DTBA), tris(2-carboxyethyl) phosphine (TCEP), or combinations thereof. A reducing agent can degrade the disulfide bonds formed between gel precursors forming the bead, and thus, degrade the bead. In other cases, a change in pH of a solution, such as an increase in pH, can trigger degradation of a bead. In other cases, exposure to an aqueous solution, such as water, can trigger hydrolytic degradation, and thus degradation of the bead. In some cases, any combination of stimuli can trigger degradation of a bead. For example, a change in pH can enable a chemical agent (e.g., DTT) to become an effective reducing agent.

Beads can also be induced to release their contents upon the application of a thermal stimulus. A change in temperature can cause a variety of changes to a bead. For example, heat can cause a solid bead to liquefy. A change in heat can cause melting of a bead such that a portion of the bead degrades. In other cases, heat can increase the internal pressure of the bead components such that the bead ruptures or explodes. Heat can also act upon heat-sensitive polymers used as materials to construct beads.

Any suitable agent can degrade beads. In some embodiments, changes in temperature or pH can be used to degrade thermo-sensitive or pH-sensitive bonds within beads. In some embodiments, chemical degrading agents can be used to degrade chemical bonds within beads by oxidation, reduction or other chemical changes. For example, a chemical degrading agent can be a reducing agent, such as DTT, wherein DTT can degrade the disulfide bonds formed between a crosslinker and gel precursors, thus degrading the bead. In some embodiments, a reducing agent can be added to degrade the bead, which may or may not cause the bead to release its contents. Examples of reducing agents can include dithiothreitol (DTT), (3-mercaptoethanol, (2S)-2-amino-1,4-dimercaptobutane (dithiobutylamine or DTBA), tris(2-carboxyethyl) phosphine (TCEP), or combinations thereof. The reducing agent can be present at a concentration of about 0.1 mM, 0.5 mM, 1 mM, 5 mM, or 10 mM. The reducing agent can be present at a concentration of at least about 0.1 mM, 0.5 mM, 1 mM, 5 mM, 10 mM, or greater than mM. The reducing agent can be present at concentration of at most about 10 mM, 5 mM, 1 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing oligonucleotide bearing beads.

Although FIG. 1 and FIG. 2 have been described in terms of providing substantially singly occupied partitions, above, in certain cases, it may be desirable to provide multiply occupied partitions, e.g., containing two, three, four or more cells and/or microcapsules (e.g., beads) including barcoded nucleic acid molecules (e.g., nucleic acid barcode molecules, oligonucleotides) within a single partition (e.g., multiomics method described elsewhere, herein). Accordingly, as noted above, the flow characteristics of the biological particle and/or bead containing fluids and partitioning fluids can be controlled to provide for such multiply occupied partitions. In particular, the flow parameters can be controlled to provide a given occupancy rate at greater than about 50% of the partitions, greater than about 75%, and in some cases greater than about 80%, 90%, 95%, or higher.

In some cases, additional microcapsules (e.g., beads) can be used to deliver additional reagents to a partition. In such cases, it can be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction can be controlled to provide for a certain ratio of microcapsules (e.g., beads) from each source, while ensuring a given pairing or combination of such microcapsules or beads into a partition with a given number of biological particles (e.g., one biological particle and one bead per partition).

The partitions described herein can include small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets can have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with microcapsules, it will be appreciated that the sample fluid volume, e.g., including co-partitioned biological particles and/or beads, within the partitions can be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species can generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions can include both unoccupied partitions (e.g., empty partitions) and occupied partitions.

Reagents

In accordance with certain aspects, biological particles can be partitioned along with lysis reagents in order to release the contents of the biological particles within the partition. See, e.g., U.S. Pat. Pub. 2018/0216162 (now U.S. Pat. No. 10,428,326), U.S. Pat. Pub. 2019/0100632 (now U.S. Pat. No. 10,590,244), and U.S. Pat. Pub. 2019/0233878, which are incorporated by reference in their entirety. Biological particles (e.g., cells, cell beads, cell nuclei, organelles, and the like) can be partitioned together with nucleic acid barcode molecules and the nucleic acid molecules of or derived from the biological particle (e.g., mRNA, cDNA, gDNA, etc.,) can be barcoded as described elsewhere herein. In some embodiments, biological particles are co-partitioned with barcode carrying beads (e.g., gel beads) and the nucleic acid molecules of or derived from the biological particle are barcoded as described elsewhere herein. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles can be partitioned along with other reagents, as will be described further below.

Beneficially, when lysis reagents and biological particles are co-partitioned, the lysis reagents can facilitate the release of the contents of the biological particles within the partition. The contents released in a partition can remain discrete from the contents of other partitions.

As will be appreciated, the channel segments described herein can be coupled to any of a variety of different fluid sources or receiving components, including reservoirs, tubing, manifolds, or fluidic components of other systems. As will be appreciated, the microfluidic channel structures can have other geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment can be controlled to control the partitioning of the different elements into droplets. Fluid can be directed flow along one or more channels or reservoirs via one or more fluid flow units. A fluid flow unit can include compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents can additionally or alternatively be co-partitioned with the biological particles to cause the release of the biological particle's contents into the partitions. For example, in some cases, surfactant-based lysis solutions can be used to lyse cells (e.g., B cell, labelled B cell, memory B cell, plasma cell), although these can be less desirable for emulsion based systems where the surfactants can interfere with stable emulsions. In some cases, lysis solutions can include non-ionic surfactants such as, for example, Triton X-100 and Tween 20. In some cases, lysis solutions can include ionic surfactants such as, for example, sarcosyl and sodium dodecyl sulfate (SDS). Electroporation, thermal, acoustic or mechanical cellular disruption can also be used in certain cases, e.g., non-emulsion based partitioning such as encapsulation of biological particles that can be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles (e.g., cells, B cells, labelled B cells, memory B cells, or plasma cells) described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated or entrained biological particles (e.g., cells, B cells, labelled B cells, memory B cells, plasma cells, or cell beads), the biological particles can be exposed to an appropriate stimulus to release the biological particles or their contents from a co-partitioned microcapsule or cell bead. For example, in some cases, a chemical stimulus can be co-partitioned along with an encapsulated biological particle to allow for the degradation of the microcapsule (e.g., encapsulating material) and release of the cell or its contents into the larger partition. In some cases, this stimulus can be the same as the stimulus described elsewhere herein for release of nucleic acid molecules (e.g., nucleic acid barcode molecules or barcoded oligonucleotides) from their respective microscapsule (e.g., bead). In alternative aspects, this can be a different and non-overlapping stimulus, in order to allow an encapsulated biological particle to be released into a partition at a different time from the release of nucleic acid molecules (e.g., nucleic acid barcode molecules or barcoded oligonucleotides) into the same partition.

Additional reagents can also be co-partitioned with the biological particles (e.g., cells, B cells, labelled B cells, memory B cells, or plasma cells), such as endonucleases to fragment a biological particle's DNA, DNA polymerase enzymes and dNTPs used to amplify the biological particle's nucleic acid fragments and to attach the barcode molecular tags to the amplified fragments. Other enzymes can be co-partitioned, including without limitation, polymerase, transposase, ligase, proteinase K, DNAse, etc. Additional reagents can also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides can include a hybridization region and a template region. The hybridization region can include any sequence capable of hybridizing to the target. In some cases, as previously described, the hybridization region includes a series of G bases to complement the overhanging C bases at the 3′ end of a cDNA molecule. The series of G bases can include 1 G base, 2 G bases, 3 G bases, 4 G bases, 5 G bases or more than 5 G bases. The template sequence can include any sequence to be incorporated into the cDNA. In some cases, the template region includes at least 1 (e.g., at least 2, 3, 4, 5 or more) tag sequences and/or functional sequences. Switch oligos can include deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo can be at least about 2, 3, 4, 5, 6, 7, 8, 9, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo can be at most about 2, 3, 4, 5, 6, 7, 8, 9, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells (e.g., B cells, labelled B cells, memory B cells, or plasma cells) are released into their respective partitions, the macromolecular components (e.g., macromolecular constituents of biological particles, such as RNA, DNA, proteins, or secreted antibodies or antigen binding fragments thereof) contained therein can be further processed within the partitions. In accordance with the methods and systems described herein, the macromolecular component contents of individual biological particles (e.g., B cells, labelled B cells, memory B cells, or plasma cells) can be provided with unique identifiers such that, upon characterization of those macromolecular components they can be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle (e.g., B cells, labelled B cells, memory B cells, or plasma cells) or groups of biological particles (e.g., B cells, labelled B cells, memory B cells, or plasma cells) with the unique identifiers, such as described above (with reference to FIGS. 1 and 2). In some aspects, the unique identifiers are provided in the form of nucleic acid molecules (e.g., nucleic acid barcode molecules, oligonucleotides) that include nucleic acid barcode sequences that can be attached to or otherwise associated with the nucleic acid contents of individual biological particle, or to other components of the biological particle, and particularly to fragments of those nucleic acids. The nucleic acid molecules (e.g., nucleic acid barcode molecules) are partitioned such that as between nucleic acid molecules in a given partition, the nucleic acid barcode sequences contained therein are the same, but as between different partitions, the nucleic acid molecule (e.g., nucleic acid barcode molecule) can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences can be present.

The nucleic acid barcode sequences can include from about 6 to about 20 or more nucleotides within the sequence of the nucleic acid molecules (e.g., nucleic acid barcode molecules, oligonucleotides). The nucleic acid barcode sequences can include from about 6 to about 20, 30, 40, 50, 60, 70, 80, 90, 100 or more nucleotides. In some cases, the length of a barcode sequence can be about 6, 7, 8, 9, 10, 11, 12, 13, 14, 15, 16, 17, 18, 19, 20 nucleotides or longer. In some cases, the length of a barcode sequence can be at least about 6, 7, 8, 9, 10, 11, 12, 13, 14, 15, 16, 17, 18, 19, 20 nucleotides or longer. In some cases, the length of a barcode sequence can be at most about 6, 7, 8, 9, 10, 11, 12, 13, 14, 15, 16, 17, 18, 19, 20 nucleotides or shorter. These nucleotides can be completely contiguous, i.e., in a single stretch of adjacent nucleotides, or they can be separated into two or more separate subsequences that are separated by 1 or more nucleotides. In some cases, separated barcode subsequences can be from about 4 to about 16 nucleotides in length. In some cases, the barcode subsequence can be about 4, 5, 6, 7, 8, 9, 10, 11, 12, 13, 14, 15, 16 nucleotides or longer. In some cases, the barcode subsequence can be at least about 4, 5, 6, 7, 8, 9, 10, 11, 12, 13, 14, 15, 16 nucleotides or longer. In some cases, the barcode subsequence can be at most about 4, 5, 6, 7, 8, 9, 10, 11, 12, 13, 14, 15, 16 nucleotides or shorter.

The co-partitioned nucleic acid molecules (e.g., nucleic acid barcode molecules) can also include other functional sequences useful in the processing of the nucleic acids from the co-partitioned biological particles (e.g., B cells, labelled B cells, memory B cells, or plasma cells). These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides can also be employed, including, e.g., coalescence of two or more droplets, where one droplet contains oligonucleotides, or microdispensing of oligonucleotides into partitions, e.g., droplets within microfluidic systems.

In an example, microcapsules, such as beads, are provided that each include large numbers of the above described barcoded nucleic acid molecules (e.g., nucleic acid barcode molecules or barcoded oligonucleotides) releasably attached to the beads, where all of the nucleic acid molecules (e.g., nucleic acid barcode molecules) attached to a particular bead will include the same nucleic acid barcode sequence, but where a large number of diverse barcode sequences are represented across the population of beads used. In some embodiments, hydrogel beads, e.g., including polyacrylamide polymer matrices, are used as a solid support and delivery vehicle for the nucleic acid molecules (nucleic acid barcode molecules) into the partitions, as they are capable of carrying large numbers of nucleic acid molecules (nucleic acid barcode molecules), and can be configured to release those nucleic acid molecules (nucleic acid barcode molecules) upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of nucleic acid molecules including the barcode sequence on an individual bead can be at least about 1,000 nucleic acid molecules, at least about 5,000 nucleic acid molecules, at least about nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences can provide greater assurance of identification in the subsequent processing, e.g., by providing a stronger address or attribution of the barcodes to a given partition, as a duplicate or independent confirmation of the output from a given partition.

The nucleic acid molecules (e.g., nucleic acid barcode molecules or oligonucleotides) are releasable from the beads upon the application of a particular stimulus to the beads. In some cases, the stimulus can be a photo-stimulus, e.g., through cleavage of a photolabile linkage that releases the nucleic acid molecules. In other cases, a thermal stimulus can be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and can be degraded for release of the attached nucleic acid molecules through exposure to a reducing agent, such as DTT.

Systems and Methods for Controlled Partitioning

In some aspects, provided are systems and methods for controlled partitioning. Droplet size can be controlled by adjusting certain geometric features in channel architecture (e.g., microfluidics channel architecture). For example, an expansion angle, width, and/or length of a channel can be adjusted to control droplet size.

FIG. 2 shows an example of a microfluidic channel structure for the controlled partitioning of beads into discrete droplets. A channel structure 200 can include a channel segment 202 communicating at a channel junction 206 (or intersection) with a reservoir 204. The reservoir 204 can be a chamber. Any reference to “reservoir,” as used herein, can also refer to a “chamber.” In operation, an aqueous fluid 208 that includes suspended beads 212 can be transported along the channel segment 202 into the junction 206 to meet a second fluid 210 that is immiscible with the aqueous fluid 208 in the reservoir 204 to create droplets 216, 218 of the aqueous fluid 208 flowing into the reservoir 204. At the junction 206 where the aqueous fluid 208 and the second fluid 210 meet, droplets can form based on factors such as the hydrodynamic forces at the junction 206, flow rates of the two fluids 208, 210, fluid properties, and certain geometric parameters (e.g., w, h₀, α, etc.) of the channel structure 200. A plurality of droplets can be collected in the reservoir 204 by continuously injecting the aqueous fluid 208 from the channel segment 202 through the junction 206.

A discrete droplet generated can include a bead (e.g., as in occupied droplets 216). Alternatively, a discrete droplet generated can include more than one bead. Alternatively, a discrete droplet generated cannot include any beads (e.g., as in unoccupied droplet 218). In some instances, a discrete droplet generated can contain one or more biological particles, as described elsewhere herein. In some instances, a discrete droplet generated can include one or more reagents, as described elsewhere herein.

In some instances, the aqueous fluid 208 can have a substantially uniform concentration or frequency of beads 212. The beads 212 can be introduced into the channel segment 202 from a separate channel (not shown in FIG. 2). The frequency of beads 212 in the channel segment 202 can be controlled by controlling the frequency in which the beads 212 are introduced into the channel segment 202 and/or the relative flow rates of the fluids in the channel segment 202 and the separate channel. In some instances, the beads can be introduced into the channel segment 202 from a plurality of different channels, and the frequency controlled accordingly.

In some instances, the aqueous fluid 208 in the channel segment 202 can include biological particles (e.g., described with reference to FIG. 1). In some instances, the aqueous fluid 208 can have a substantially uniform concentration or frequency of biological particles. As with the beads, the biological particles (e.g., B cells, labelled B cells, memory B cells, or plasma cells) can be introduced into the channel segment 202 from a separate channel. The frequency or concentration of the biological particles in the aqueous fluid 208 in the channel segment 202 can be controlled by controlling the frequency in which the biological particles are introduced into the channel segment 202 and/or the relative flow rates of the fluids in the channel segment 202 and the separate channel. In some instances, the biological particles can be introduced into the channel segment 202 from a plurality of different channels, and the frequency controlled accordingly. In some instances, a first separate channel can introduce beads and a second separate channel can introduce biological particles into the channel segment 202. The first separate channel introducing the beads can be upstream or downstream of the second separate channel introducing the biological particles.

The second fluid 210 can include an oil, such as a fluorinated oil, that includes a fluorosurfactant for stabilizing the resulting droplets, for example, inhibiting subsequent coalescence of the resulting droplets.

In some instances, the second fluid 210 cannot be subjected to and/or directed to any flow in or out of the reservoir 204. For example, the second fluid 210 can be substantially stationary in the reservoir 204. In some instances, the second fluid 210 can be subjected to flow within the reservoir 204, but not in or out of the reservoir 204, such as via application of pressure to the reservoir 204 and/or as affected by the incoming flow of the aqueous fluid 208 at the junction 206. Alternatively, the second fluid 210 can be subjected and/or directed to flow in or out of the reservoir 204. For example, the reservoir 204 can be a channel directing the second fluid 210 from upstream to downstream, transporting the generated droplets.

The channel structure 200 at or near the junction 206 can have certain geometric features that at least partly determine the sizes of the droplets formed by the channel structure 200. The channel segment 202 can have a height, h₀ and width, w, at or near the junction 206. By way of example, the channel segment 202 can include a rectangular cross-section that leads to a reservoir 204 having a wider cross-section (such as in width or diameter). Alternatively, the cross-section of the channel segment 202 can be other shapes, such as a circular shape, trapezoidal shape, polygonal shape, or any other shapes. The top and bottom walls of the reservoir 204 at or near the junction 206 can be inclined at an expansion angle, a. The expansion angle, a, allows the tongue (portion of the aqueous fluid 208 leaving channel segment 202 at junction 206 and entering the reservoir 204 before droplet formation) to increase in depth and facilitate decrease in curvature of the intermediately formed droplet. Droplet size can decrease with increasing expansion angle. The resulting droplet radius, R_{d}, can be predicted by the following equation for the aforementioned geometric parameters of h₀, w, and α:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 μm, h=25 μm, and α=5°, the predicted droplet size is 123 μm. In another example, for a channel structure with w=28 μm, h=28 μm, and α=7°, the predicted droplet size is 124 μm.

In some instances, the expansion angle, a, can be between a range of from about to about 4°, from about 0.1° to about 10°, or from about 0° to about 90°. For example, the expansion angle can be at least about 0.010, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, iv, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 208 entering the junction 206 can be between about 0.04 microliters (μL)/minute (min) and about 40 μL/min. In some instances, the flow rate of the aqueous fluid 208 entering the junction 206 can be between about 0.01 microliters (μL)/minute (min) and about 100 μL/min. Alternatively, the flow rate of the aqueous fluid 208 entering the junction 206 can be less than about 0.01 μL/min. Alternatively, the flow rate of the aqueous fluid 208 entering the junction 206 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140, 150 μL/min, or greater. At lower flow rates, such as flow rates of about less than or equal to 10 microliters/minute, the droplet radius cannot be dependent on the flow rate of the aqueous fluid 208 entering the junction 206.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 206) between aqueous fluid 208 channel segments (e.g., channel segment 202) and the reservoir 204. Alternatively or in addition, the throughput of droplet generation can be increased by increasing the flow rate of the aqueous fluid 208 in the channel segment 202.

The methods and systems described herein can be used to greatly increase the efficiency of single cell applications and/or other applications receiving droplet-based input.

Subsequent operations that can be performed (for example, following the sorting of occupied cells and/or appropriately sized cells) can include generation of amplification products, purification (e.g., via solid phase reversible immobilization (SPRI)), further processing (e.g., shearing, ligation of functional sequences, and subsequent amplification (e.g., via PCR)). These operations can occur in bulk (e.g., outside the partition). In the case where a partition is a droplet in an emulsion, the emulsion can be broken and the contents of the droplet pooled for additional operations. Additional reagents that can be co-partitioned along with the barcode bearing bead can include oligonucleotides to block ribosomal RNA (rRNA) and nucleases to digest genomic DNA from cells. Alternatively, rRNA removal agents can be applied during additional processing operations. The configuration of the constructs generated by such a method can help minimize (or avoid) sequencing of the poly-T sequence during sequencing and/or sequence the end of a polynucleotide sequence. The amplification products, for example, first amplification products and/or second amplification products, can be subject to sequencing for sequence analysis. In some cases, amplification can be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

Partitions including a barcode bead (e.g., a gel bead) associated with barcode molecules and a bead encapsulating cellular constituents (e.g., a cell bead) such as cellular nucleic acids can be useful in constituent analysis as is described in U.S. Patent Publication No. 2018/0216162, which is herein incorporated by reference in its entirety for all purposes.

Sample and Cell Processing

A sample can be derived from any useful source including any subject, such as a human subject. A sample can include material (e.g., one or more cells) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, can be obtained for analysis as described herein. For example, a first sample can be obtained from a subject at a first time and a second sample can be obtained from the subject at a second time later than the first time. The first time can be before a subject undergoes a treatment regimen or procedure (e.g., to address a disease or condition), and the second time can be during or after the subject undergoes the treatment regimen or procedure. In another example, a first sample can be obtained from a first bodily location or system of a subject (e.g., using a first collection technique) and a second sample can be obtained from a second bodily location or system of the subject (e.g., using a second collection technique), which second bodily location or system can be different than the first bodily location or system. In another example, multiple samples can be obtained from a subject at a same time from the same or different bodily locations. Different samples, such as different samples collected from different bodily locations of a same subject, at different times, from multiple different subjects, and/or using different collection techniques, can undergo the same or different processing (e.g., as described herein). For example, a first sample can undergo a first processing protocol and a second sample can undergo a second processing protocol.

A sample can be a biological sample, such as a cell sample (e.g., as described herein). A sample can include one or more biological particles, e.g., one of more analyte carriers such as one or more cells and/or cellular constituents, such as one or more cell nuclei. For example, a sample can include a plurality of cells and/or cellular constituents. Components (e.g., cells or cellular constituents, such as cell nuclei) of a sample can be of a single type or a plurality of different types. For example, cells of a sample can include one or more different types of blood cells.

A biological sample can include a plurality of cells having different dimensions and features. In some cases, processing of the biological sample, such as cell separation and sorting (e.g., as described herein), can affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process can include the use of microfluidics (e.g., to separate biological particles (e.g., analyte carriers), of different sizes, types, charges, or other features).

In an example, a sample including one or more cells can be processed to separate the one or more cells from other materials in the sample (e.g., using centrifugation and/or another process). In some cases, cells and/or cellular constituents of a sample can be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process can include a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells).

Separation of one or more different types of cells can include, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method. For example, a flow cytometry method can be used to detect cells and/or cellular constituents based on a parameter such as a size, morphology, or protein expression. Flow cytometry-based cell sorting can include injecting a sample into a sheath fluid that conveys the cells and/or cellular constituents of the sample into a measurement region one at a time. In the measurement region, a light source such as a laser can interrogate the cells and/or cellular constituents and scattered light and/or fluorescence can be detected and converted into digital signals. A nozzle system (e.g., a vibrating nozzle system) can be used to generate droplets (e.g., aqueous droplets) including individual cells and/or cellular constituents. Droplets including cells and/or cellular constituents of interest (e.g., as determined via optical detection) can be labeled with an electric charge (e.g., using an electrical charging ring), which charge can be used to separate such droplets from droplets including other cells and/or cellular constituents. For example, FACS can include labeling cells and/or cellular constituents with fluorescent markers (e.g., using internal and/or external biomarkers). Cells and/or cellular constituents can then be measured and identified one by one and sorted based on the emitted fluorescence of the marker or absence thereof. MACS can use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS can use microbubbles (e.g., glass microbubbles) labeled with antibodies to target cells of interest. Cells and/or cellular components coupled to microbubbles can float to a surface of a solution, thereby separating target cells and/or cellular components from other components of a sample. Cell separation techniques can be used to enrich for populations of cells of interest (e.g., prior to partitioning, as described herein). For example, a sample including a plurality of cells including a plurality of cells of a given type can be subjected to a positive separation process. The plurality of cells of the given type can be labeled with a fluorescent marker (e.g., based on an expressed cell surface marker or another marker) and subjected to a FACS process to separate these cells from other cells of the plurality of cells. The selected cells can then be subjected to subsequent partition-based analysis (e.g., as described herein) or other downstream analysis. The fluorescent marker can be removed prior to such analysis or can be retained. The fluorescent marker can include an identifying feature, such as a nucleic acid barcode sequence and/or unique molecular identifier.

In another example, a first sample including a first plurality of cells including a first plurality of cells of a given type (e.g., immune cells expressing a particular marker or combination of markers) and a second sample including a second plurality of cells including a second plurality of cells of the given type can be subjected to a positive separation process. The first and second samples can be collected from the same or different subjects, at the same or different types, from the same or different bodily locations or systems, using the same or different collection techniques. For example, the first sample can be from a first subject and the second sample can be from a second subject different than the first subject. The first plurality of cells of the first sample can be provided a first plurality of fluorescent markers configured to label the first plurality of cells of the given type. The second plurality of cells of the second sample can be provided a second plurality of fluorescent markers configured to label the second plurality of cells of the given type. The first plurality of fluorescent markers can include a first identifying feature, such as a first barcode, while the second plurality of fluorescent markers can include a second identifying feature, such as a second barcode, that is different than the first identifying feature. The first plurality of fluorescent markers and the second plurality of fluorescent markers can fluoresce at the same intensities and over the same range of wavelengths upon excitation with a same excitation source (e.g., light source, such as a laser). The first and second samples can then be combined and subjected to a FACS process to separate cells of the given type from other cells based on the first plurality of fluorescent markers labeling the first plurality of cells of the given type and the second plurality of fluorescent markers labeling the second plurality of cells of the given type. Alternatively, the first and second samples can undergo separate FACS processes and the positively selected cells of the given type from the first sample and the positively selected cells of the given type from the second sample can then be combined for subsequent analysis. The encoded identifying features of the different fluorescent markers can be used to identify cells originating from the first sample and cells originating from the second sample. For example, the first and second identifying features can be configured to interact (e.g., in partitions, as described herein) with nucleic acid barcode molecules (e.g., as described herein) to generate barcoded nucleic acid products detectable using, e.g., nucleic acid sequencing.

In 620b, the bead includes nucleic acid barcode molecules that are releasably attached thereto, as described below. The bead can degrade or otherwise release the nucleic acid barcode molecules into the well 602; the nucleic acid barcode molecules can then be used to barcode nucleic acid molecules within the well 602. Further processing can be performed either inside the partition or outside the partition. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 650, further characterization, such as sequencing can be performed to generate sequencing reads. The sequencing reads can yield information on individual cells or populations of cells, which can be represented visually or graphically, e.g., in a plot.

Multiplexing Methods

In some embodiments of the disclosure, steps (a) and (b) of the methods described herein are performed in multiplex format. For example, in some embodiments, step (a) of the methods disclosed herein can include individually partitioning additional single cells (e.g., B cells, T cells) of the plurality of cells in additional partitions of the first plurality of partitions, and step (b) can further include determining all or a part of the nucleic acid sequences encoding antibodies produced by the additional single cells (e.g., B cells, T cells) or antigen-binding fragment thereof.

Accordingly, in some embodiments, the present disclosure provides methods and systems for multiplexing, and otherwise increasing throughput of samples for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cells or cell features can be used to characterize cells and/or cell features. In some instances, cell features include cell surface features. Cell surface features can include, but are not limited to, a receptor, an antigen or antigen fragment (e.g., an antigen or antigen fragment that binds to an antigen-binding molecule located on a cell surface), a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features can include intracellular analytes, such as proteins, protein modifications (e.g., phosphorylation status or other post-translational modifications), nuclear proteins, nuclear membrane proteins, or any combination thereof. A labelling agent can include, but is not limited to, a protein, a peptide, an antibody (or an epitope binding fragment thereof), an antigen, an antigen fragment, a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a Darpin, and a protein scaffold, or any combination thereof. The labelling agents can include (e.g., are attached to) a reporter oligonucleotide that is indicative of the cell surface feature to which the binding group binds. For example, the reporter oligonucleotide can include a barcode sequence that permits identification of the labelling agent. For example, a labelling agent that is specific to one type of cell feature (e.g., a first cell surface feature) can have a first reporter oligonucleotide coupled thereto, while a labelling agent that is specific to a different cell feature (e.g., a second cell surface feature) can have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, each of which is herein entirely incorporated by reference for all purposes.

In a particular example, a library of potential cell feature labelling agents can be provided, where the respective cell feature labelling agents are associated with nucleic acid reporter molecules, such that a different reporter oligonucleotide sequence is associated with each labelling agent capable of binding to a specific cell feature. In other aspects, different members of the library can be characterized by the presence of a different oligonucleotide sequence label. For example, an antibody capable of binding to a first protein can have associated with it a first reporter oligonucleotide sequence, while an antibody, (which may be the same antibody), capable of binding to a second protein can have a different, (or additional if the same antibody), reporter oligonucleotide sequence associated with it. The presence of the particular oligonucleotide sequence can be indicative of the presence of a particular antibody or cell feature which can be recognized or bound by the particular antibody.

Labelling agents capable of binding to or otherwise coupling to one or more cells can be used to characterize a cell as belonging to a particular set of cells. For example, labeling agents can be used to label a sample of cells or a group of cells, e.g., to provide a sample index. For other example, labelling agents can be used to label a group of cells belonging to a particular experimental condition. In this way, a group of cells can be labeled as different from another group of cells. In an example, a first group of cells can originate from a first sample and a second group of cells can originate from a second sample. Labelling agents can allow the first group and second group to have a different labeling agent (or reporter oligonucleotide associated with the labeling agent). This can, for example, facilitate multiplexing, where cells of the first group and cells of the second group can be labeled separately and then pooled together for downstream analysis. The downstream detection of a label can indicate analytes as belonging to a particular group.

For example, a reporter oligonucleotide can be linked to an antibody or an epitope binding fragment thereof, and labeling a cell can include subjecting the antibody-linked barcode molecule or the epitope binding fragment-linked barcode molecule to conditions suitable for binding the antibody to a molecule present on a surface of the cell. The binding affinity between the antibody or the epitope binding fragment thereof and the molecule present on the surface can be within a desired range to ensure that the antibody or the epitope binding fragment thereof remains bound to the molecule. For example, the binding affinity can be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds can be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant can be less than about 10 μM. In some embodiments, the antibody or antigen-binding fragment thereof has a desired dissociation rate constant (koff), such that the antibody or antigen binding fragment thereof remains bound to the molecule during various sample processing steps.

In another example, a reporter oligonucleotide can be coupled to a cell-penetrating peptide (CPP), and labeling cells can include delivering the CPP coupled reporter oligonucleotide into a biological particle (e.g., analyte carrier). Labeling biological particles (e.g., analyte carriers) can include delivering the CPP conjugated oligonucleotide into a cell and/or cell bead by the cell-penetrating peptide. A CPP that can be used in the methods provided herein can include at least one non-functional cysteine residue, which can be either free or derivatized to form a disulfide link with an oligonucleotide that has been modified for such linkage. Non-limiting examples of CPPs that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP can be an arginine-rich peptide transporter. The CPP can be Penetratin or the Tat peptide. In another example, a reporter oligonucleotide can be coupled to a fluorophore or dye, and labeling cells can include subjecting the fluorophore-linked barcode molecule to conditions suitable for binding the fluorophore to the surface of the cell. In some instances, fluorophores can interact strongly with lipid bilayers and labeling cells can include subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, which is hereby incorporated by reference in its entirety for all purposes, for a description of organic fluorophores.

A reporter oligonucleotide can be coupled to a lipophilic molecule, and labeling cells can include delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane can be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide can enter into the intracellular space and/or a cell nucleus. In some embodiments, a reporter oligonucleotide coupled to a lipophilic molecule will remain associated with and/or inserted into lipid membrane (as described herein) via the lipophilic molecule until lysis of the cell occurs, e.g., inside a partition. Exemplary embodiments of lipophilic molecules coupled to reporter oligonucleotides are described in PCT/US2018/064600, which is hereby incorporated by reference in its entirety.

A reporter oligonucleotide can be part of a nucleic acid molecule including any number of functional sequences, as described elsewhere herein, such as a target capture sequence, a random primer sequence, and the like, and coupled to another nucleic acid molecule that is, or is derived from, the analyte.

Prior to partitioning, the cells can be incubated with the library of labelling agents, that can be labelling agents to a broad panel of different cell features, e.g., receptors, proteins, etc., and which include their associated reporter oligonucleotides. Unbound labelling agents can be washed from the cells, and the cells can then be co-partitioned (e.g., into droplets or wells) along with partition-specific barcode oligonucleotides (e.g., attached to a support, such as a bead or gel bead) as described elsewhere herein. As a result, the partitions can include the cell or cells, as well as the bound labelling agents and their known, associated reporter oligonucleotides.

In other instances, e.g., to facilitate sample multiplexing, a labelling agent that is specific to a particular cell feature can have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent can interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088, which is hereby entirely incorporated by reference for all purposes.

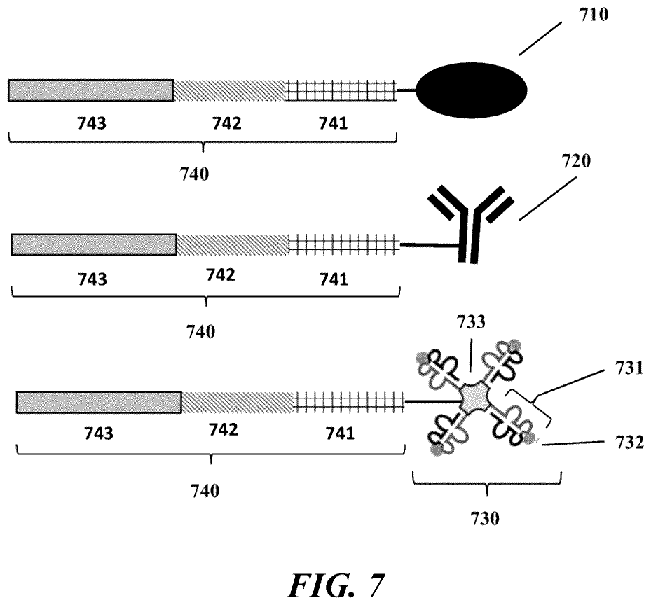

In some embodiments, to facilitate sample multiplexing, individual samples can be stained with lipid tags, such as cholesterol-modified oligonucleotides (CMOs, see, e.g., FIG. 7), anti-calcium channel antibodies, or anti-ACTB antibodies. Non-limiting examples of anti-calcium channel antibodies include anti-KCNN4 antibodies, anti-BK channel beta 3 antibodies, anti-a1B calcium channel antibodies, and anti-CACNA1A antibodies. Examples of anti-ACTB antibodies suitable for the methods of the disclosure include, but are not limited to, mAbGEa, ACTN05, AC-15, 15G5A11/E2, BA3R, and HHF35.

As described elsewhere herein, libraries of labelling agents can be associated with a particular cell feature as well as be used to identify analytes as originating from a particular cell population, or sample. Cell populations can be incubated with a plurality of libraries such that a cell or cells include multiple labelling agents. For example, a cell can include coupled thereto a lipophilic labeling agent and an antibody. The lipophilic labeling agent can indicate that the cell is a member of a particular cell sample, whereas the antibody can indicate that the cell includes a particular analyte. In this manner, the reporter oligonucleotides and labelling agents can allow multi-analyte, multiplexed analyses to be performed.

In some instances, these reporter oligonucleotides can include nucleic acid barcode sequences that permit identification of the labelling agent which the reporter oligonucleotide is coupled to. The use of oligonucleotides as the reporter can provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents can be achieved through any of a variety of direct or indirect, covalent or non-covalent associations or attachments. For example, oligonucleotides (e.g., reporter oligonucleotides) can be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or a streptavidin linker in monomeric or multimeric form (e.g., tetramic form of streptavidin). Those of skill in the art will recognize that a streptavidin monomer encompasses streptavidin molecules with 1 biotin binding site, while a streptavidin multimer encompasses strepatavidin molecules with more than 1 biotin binding site. For example, a streptavidin tetramer has 4 biotin binding sites. However, a skilled artisan will also recognize that in a streptavidin tetramer does not necessarily comprise 4 streptavidins complexed together. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, can be used to couple reporter oligonucleotides to labelling agents. Commercially available kits, such as those from Thunderlink and Abcam, and techniques common in the art can be used to couple reporter oligonucleotides to labelling agents as appropriate. In another example, a labelling agent is indirectly (e.g., via hybridization) coupled to a reporter oligonucleotide including a barcode sequence that identifies the label agent. For instance, the labelling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can include a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide can be allowed to hybridize to the reporter oligonucleotide.

FIG. 7 describes exemplary labelling agents (710, 720, 730) including reporter oligonucleotides (740) attached thereto. Labelling agent 710 (e.g., any of the labelling agents described herein) is attached (either directly, e.g., covalently attached, or indirectly) to reporter oligonucleotide 740. Reporter oligonucleotide 740 can include barcode sequence 742 that identifies labelling agent 710. Reporter oligonucleotide 740 can also include one or more functional sequences 743 that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 7, in some instances, reporter oligonucleotide 740 conjugated to a labelling agent (e.g., 710, 720, 730) includes a functional sequence 741, a reporter barcode sequence 742 that identifies the labelling agent (e.g., 710, 720, 730), and reporter capture handle 743. Reporter capture handle sequence 743 can be configured to hybridize to a complementary sequence, such as a complementary sequence present on a nucleic acid barcode molecule, such as those described elsewhere herein. In some instances, nucleic acid barcode molecule is attached to a support (e.g., a bead, such as a gel bead), such as those described elsewhere herein (e.g., FIGS. 3, 4, 8 and 9A-9B). For example, nucleic acid barcode molecule can be attached to the support via a releasable linkage (e.g., including a labile bond), such as those described elsewhere herein (e.g., FIGS. 3, 4, 8 and 9A-9B). In some instances, reporter oligonucleotide 740 includes one or more additional functional sequences, such as those described above.

In some instances, the labelling agent 710 is a protein or polypeptide (e.g., an antigen or prospective antigen) including reporter oligonucleotide 740. Reporter oligonucleotide 740 includes reporter barcode sequence 742 that identifies polypeptide 710 and can be used to infer the presence of an analyte, e.g., a binding partner of polypeptide 710 (i.e., a molecule or compound to which polypeptide 710 can bind). In some instances, the labelling agent 710 is a lipophilic moiety (e.g., cholesterol) including reporter oligonucleotide 740, where the lipophilic moiety is selected such that labelling agent 710 integrates into a membrane of a cell or nucleus. Reporter oligonucleotide 740 includes reporter barcode sequence 742 that identifies lipophilic moiety 710 which in some instances is used to tag cells (e.g., groups of cells, cell samples, etc.) and can be used for multiplex analyses as described elsewhere herein. In some instances, the labelling agent is an antibody 720 (or an epitope binding fragment thereof) including reporter oligonucleotide 740. Reporter oligonucleotide 740 includes reporter barcode sequence 742 that identifies antibody 720 and can be used to infer the presence of, e.g., a target of antibody 720 (i.e., a molecule or compound to which antibody 720 binds). In other embodiments, labelling agent 730 includes an MHC molecule 731 including peptide 732 and reporter oligonucleotide 740 that identifies peptide 732. In some instances, the MHC molecule is coupled to a support 733. In some instances, support 733 can be a polypeptide, such as streptavidin, or a polysaccharide, such as dextran. In some instances, reporter oligonucleotide 740 can be directly or indirectly coupled to MHC labelling agent 730 in any suitable manner. For example, reporter oligonucleotide 740 can be coupled to MHC molecule 731, support 733, or peptide 732. In some embodiments, labelling agent 730 includes a plurality of WIC molecules, (e.g. is an WIC multimer, which can be coupled to a support (e.g., 733)). There are many possible configurations of Class I and/or Class II WIC multimers that can be utilized with the compositions, methods, and systems disclosed herein, e.g., WIC tetramers, MHC pentamers (MEW assembled via a coiled-coil domain, e.g., Pro5® WIC Class I Pentamers, (ProImmune, Ltd.), WIC octamers, WIC dodecamers, MHC decorated dextran molecules (e.g., WIC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969, each of which is herein entirely incorporated by reference for all purposes.

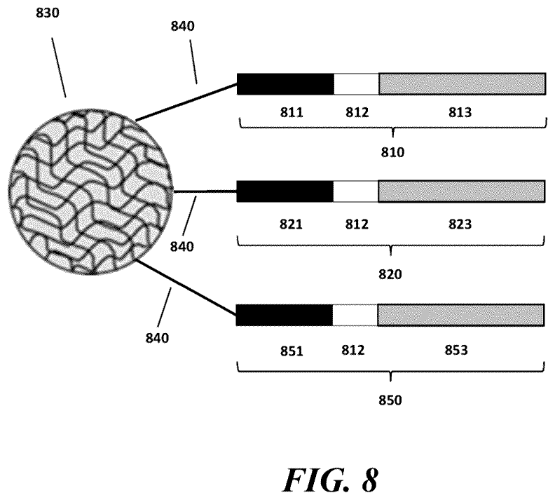

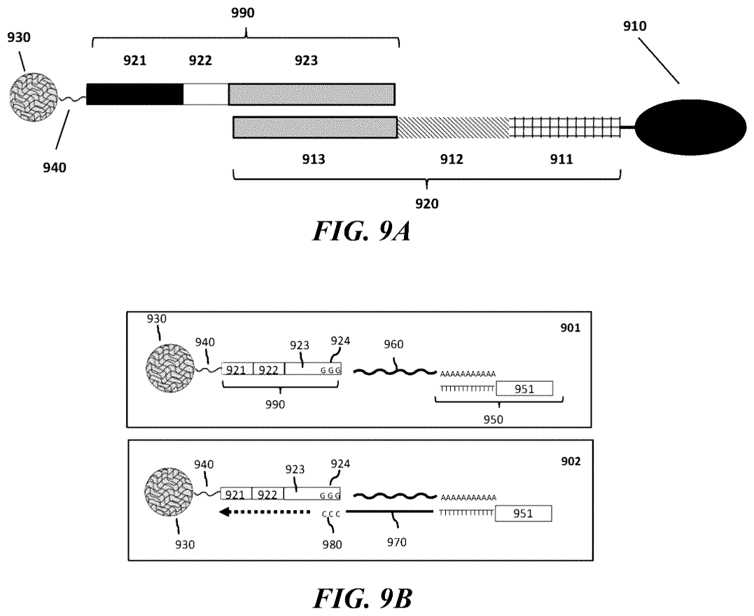

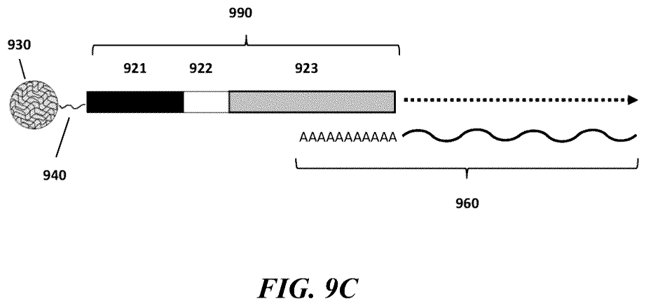

Exemplary barcode molecules attached to a support (e.g., a bead) is shown in FIG. 8. In some embodiments, analysis of multiple analytes (e.g., RNA and one or more analytes using labelling agents described herein) can include nucleic acid barcode molecules as generally depicted in FIG. 8. In some embodiments, nucleic acid barcode molecules 810 and 820 are attached to support 830 via a releasable linkage 840 (e.g., including a labile bond) as described elsewhere herein. Nucleic acid barcode molecule 810 can include functional sequence 811, barcode sequence 812 and capture sequence 813. Nucleic acid barcode molecule 820 can include adapter sequence 821, barcode sequence 812, and adapter sequence 823, wherein adapter sequence 823 includes a different sequence than adapter sequence 813. In some instances, adapter 811 and adapter 821 include the same sequence. In some instances, adapter 811 and adapter 821 include different sequences. Although support 830 is shown including nucleic acid barcode molecules 810 and 820, any suitable number of barcode molecules including common barcode sequence 812 are contemplated herein. For example, in some embodiments, support 830 further includes nucleic acid barcode molecule 850. Nucleic acid barcode molecule 850 can include adapter sequence 851, barcode sequence 812 and adapter sequence 853, wherein adapter sequence 853 includes a different sequence than adapter sequence 813 and 823. In some instances, nucleic acid barcode molecules (e.g., 810, 820, 850) include one or more additional functional sequences, such as a UMI or other sequences described herein. The nucleic acid barcode molecules 810, 820 or 850 can interact with analytes as described elsewhere herein, for example, as depicted in FIGS. 9A-9C.

Referring to FIG. 9A, in an instance where cells are labelled with labeling agents, capture sequence 923 can be complementary to an adapter sequence of a reporter oligonucleotide. Cells can be contacted with one or more reporter oligonucleotide 920 conjugated labelling agents 910 (e.g., polypeptide, antibody, or others described elsewhere herein). In some cases, the cells can be further processed prior to barcoding. For example, such processing steps can include one or more washing and/or cell sorting steps. In some instances, a cell that is bound to labelling agent 910 which is conjugated to reporter oligonucleotide 920 and support 930 (e.g., a bead, such as a gel bead) including nucleic acid barcode molecule 990 is partitioned into a partition amongst a plurality of partitions (e.g., a droplet of a droplet emulsion or a well of a microwell array). In some instances, the partition includes at most a single cell bound to labelling agent 910. In some instances, reporter oligonucleotide 920 conjugated to labelling agent 910 (e.g., polypeptide, an antibody, pMHC molecule such as an MHC multimer, etc.) includes a first adapter sequence 911 (e.g., a primer sequence), a barcode sequence 912 that identifies the labelling agent 910 (e.g., the polypeptide, antibody, or peptide of a pMHC molecule or complex), and an adapter (e.g., capture handle) sequence 913. Adapter (e.g., capture handle) sequence 913 can be configured to hybridize to a complementary sequence, such as capture sequence 923 present on a nucleic acid barcode molecule 990 (e.g., partition-specific barcode molecule). In some instances, reporter oligonucleotide 920 includes one or more additional functional sequences, such as those described elsewhere herein.

Barcoded nucleic acid molecules can be generated (e.g., via a nucleic acid reaction, such as nucleic acid extension, reverse transcription, or ligation) from the constructs described in FIGS. 9A-9C. For example, sequence 913 (e.g., capture handle sequence) can then be hybridized to complementary capture sequence 923 to generate (e.g., via a nucleic acid reaction, such as nucleic acid extension or ligation) a barcoded nucleic acid molecule including cell barcode (e.g., common barcode or partition-specific barcode) sequence 922 (or a reverse complement thereof) and reporter barcode sequence 912 (or a reverse complement thereof). In some embodiments, the nucleic acid barcode molecule 990 (e.g., partition-specific barcode molecule) further includes a UMI Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) can be performed. For example, the workflow can include a workflow as generally depicted in any of FIGS. 9A-9C, or a combination of workflows for an individual analyte, as described elsewhere herein. For example, by using a combination of the workflows as generally depicted in FIGS. 9A-9C, multiple analytes can be analyzed.

In some instances, analysis of an analyte (e.g. a nucleic acid, a polypeptide, a carbohydrate, a lipid, etc.) includes a workflow as generally depicted in FIG. 9A. A nucleic acid barcode molecule 990 can be co-partitioned with the one or more analytes. In some instances, nucleic acid barcode molecule 990 is attached to a support 930 (e.g., a bead, such as a gel bead), such as those described elsewhere herein. For example, nucleic acid barcode molecule 990 can be attached to support 930 via a releasable linkage 940 (e.g., including a labile bond), such as those described elsewhere herein. Nucleic acid barcode molecule 990 can include a functional sequence 921 and optionally include other additional sequences, for example, a barcode sequence 922 (e.g., common barcode, partition-specific barcode, or other functional sequences described elsewhere herein, and/or a UMI sequence. The nucleic acid barcode molecule 990 can include a capture sequence 923 that can be complementary to another nucleic acid sequence, such that it can hybridize to a particular sequence.

For example, capture sequence 923 can include a poly-T sequence and can be used to hybridize to mRNA. Referring to FIG. 9C, in some embodiments, nucleic acid barcode molecule 990 includes capture sequence 923 complementary to a sequence of RNA molecule 960 from a cell. In some instances, capture sequence 923 includes a sequence specific for an RNA molecule. Capture sequence 923 can include a known or targeted sequence or a random sequence. In some instances, a nucleic acid extension reaction can be performed, thereby generating a barcoded nucleic acid product including capture sequence 923, the barcode sequence 921, UMI sequence 922, any other functional sequence, and a sequence corresponding to the RNA molecule 960.

In another example, capture sequence 923 can be complementary to an overhang sequence or an adapter sequence that has been appended to an analyte. For example, referring to FIG. 9B, in some embodiments, primer 950 includes a sequence complementary to a sequence of nucleic acid molecule 960 (such as an RNA encoding for a BCR sequence) from a biological particle (e.g., analyte carrier). In some instances, primer 950 includes one or more sequences 951 that are not complementary to RNA molecule 960. Sequence 951 can be a functional sequence as described elsewhere herein, for example, an adapter sequence, a sequencing primer sequence, or a sequence the facilitates coupling to a flow cell of a sequencer. In some instances, primer 950 includes a poly-T sequence. In some instances, primer 950 includes a sequence complementary to a target sequence in an RNA molecule. In some instances, primer 950 includes a sequence complementary to a region of an immune molecule, such as the constant region of a TCR or BCR sequence. Primer 950 is hybridized to nucleic acid molecule 960 and complementary molecule 970 is generated. For example, complementary molecule 970 can be cDNA generated in a reverse transcription reaction. In some instances, an additional sequence can be appended to complementary molecule 970. For example, the reverse transcriptase enzyme can be selected such that several non-templated bases 980 (e.g., a poly-C sequence) are appended to the cDNA. In another example, a terminal transferase can also be used to append the additional sequence. Nucleic acid barcode molecule 990 includes a sequence 924 complementary to the non-templated bases, and the reverse transcriptase performs a template switching reaction onto nucleic acid barcode molecule 990 to generate a barcoded nucleic acid molecule including cell (e.g., partition specific) barcode sequence 922 (or a reverse complement thereof) and a sequence of complementary molecule 970 (or a portion thereof). In some instances, capture sequence 923 includes a sequence complementary to a region of an immune molecule, such as the constant region of a TCR or BCR sequence. Capture sequence 923 is hybridized to nucleic acid molecule 960 and a complementary molecule 970 is generated. For example, complementary molecule 970 can be generated in a reverse transcription reaction generating a barcoded nucleic acid molecule including cell barcode (e.g., common barcode or partition-specific barcode) sequence 922 (or a reverse complement thereof) and a sequence of complementary molecule 970 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969.

In some embodiments, biological particles (e.g., cells, nuclei) from a plurality of samples (e.g., a plurality of subjects) can be pooled, sequenced, and demultiplexed by identifying mutational profiles associated with individual samples and mapping sequence data from single biological particles to their source based on their mutational profile. See, e.g., Xu J. et al., Genome Biology Vol. 20, 290 (2019); Huang Y. et al., Genome Biology Vol. 20, 273 (2019); and Heaton et al., Nature Methods volume 17, pages 615-620(2020), which are hereby incorporated by reference in their entirety.

Gene expression data can reflect the underlying genome and mutations and structural variants therein. As a result, the variation inherent in the captured and sequenced RNA molecules can be used to identify genotypes de novo or used to assign molecules to genotypes that were known a priori. In some embodiments, allelic variation that is present due to haplotypic states (including linkage disequilibrium of the human leucocyte antigen loci (HLA), immune receptor loci (e.g., BCR), and other highly polymorphic regions of the genome), can also be used for demultiplexing. Expressed B cell receptors can be used to infer germline alleles from unrelated individuals, which information may be used for demultiplexing.

### K. Systems and Methods for Spatial Analysis

In some embodiments of methods disclosed herein that include contacting a tumor sample with a composition including one or more barcoded antibodies and/or functional fragments thereof (e.g., a barcoded antibody cocktail), the tumor sample is an intact tissue sample. For example, the intact tissue sample can be a tumor biopsy sample. In some embodiments, the intact tissue sample is a tissue section. The tissue section can be a fresh frozen tissue section, a fixed tissue section, or an FFPE tissue section. In some embodiments, the intact tissue sample is fixed and/or stained (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663. In some embodiments, the intact tissue sample is subjected to spatial analysis. Systems and methods for spatial analysis are disclosed herein.

Non-limiting aspects of spatial analysis methodologies and compositions are described in U.S. Pat. Nos. 10,774,374, 10,724,078, 10,480,022, 10,059,990, 10,041,949, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 2017; and Gupta et al., Nature Biotechnol. 36:1197-1202, 2018; the Visium Spatial Gene Expression Reagent Kits User Guide (e.g., Rev D, dated October 2020), the Visium Spatial Gene Expression for FFPE User Guide (e.g., Rev A, dated June 2021), and/or the Visium Spatial Tissue Optimization Reagent Kits User Guide (e.g., Rev D, dated October 2020), both of which are available at the 10×Genomics Support Documentation website, and can be used herein in any combination. Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. Further non-limiting aspects of spatial analysis methodologies and compositions are described herein.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

FIG. 14 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 14 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

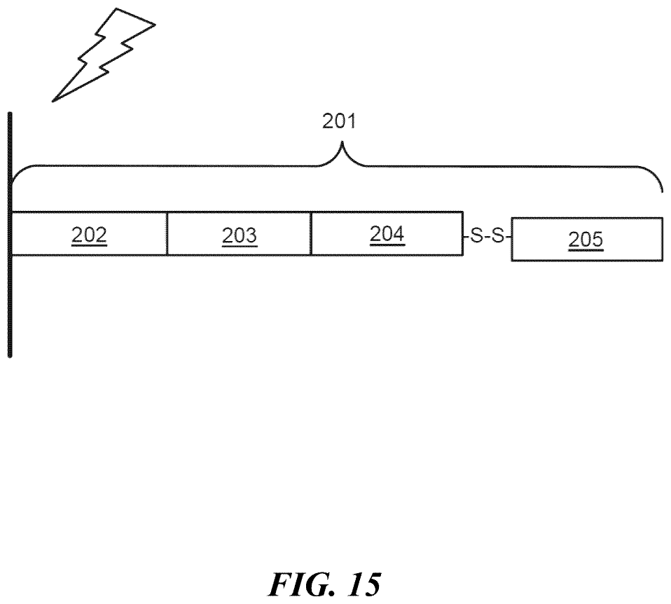

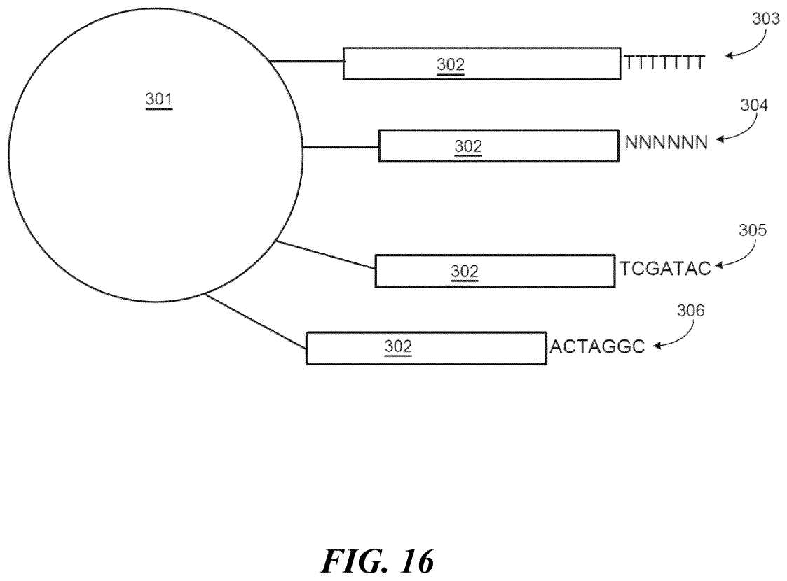

FIG. 15 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain. FIG. 16 is a schematic diagram of an exemplary multiplexed spatially-barcoded feature. In FIG. 16, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9).

While only four different capture probe-barcoded constructs are shown in FIG. 16, capture-probe barcoded constructs can be tailored for analyses of any given analyte associated with a nucleic acid and capable of binding with such a construct. For example, the schemes shown in FIG. 16 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MEW multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

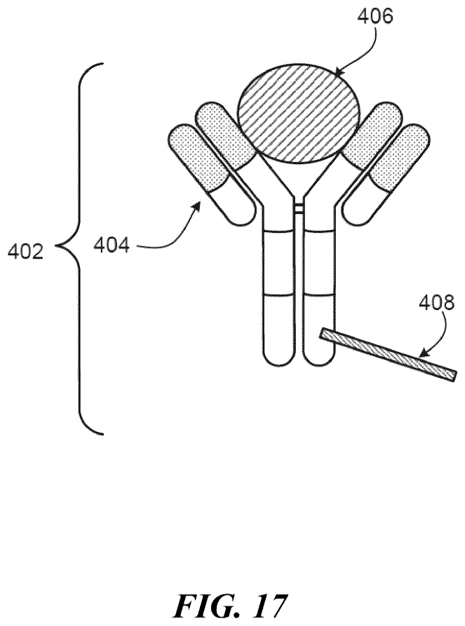

FIG. 17 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

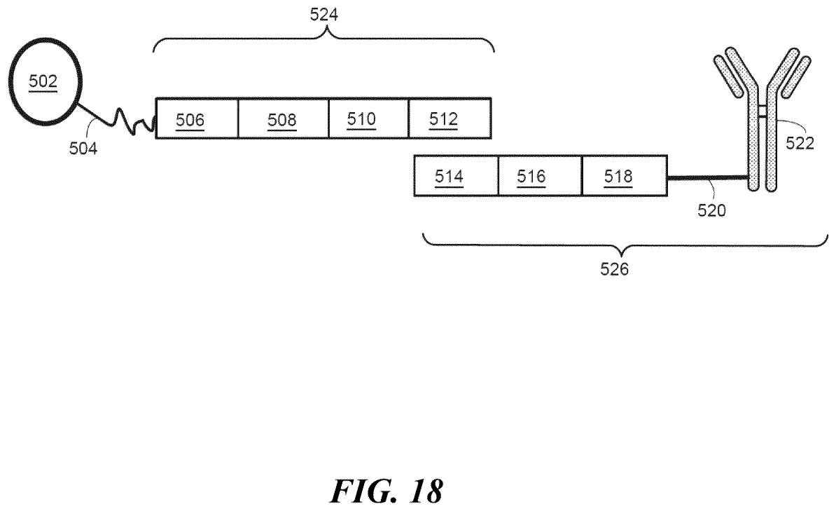

FIG. 18 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template. As used herein, an “extended capture probe” refers to a capture probe having additional nucleotides added to the terminus (e.g., 3′ or end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe. In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction). Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease. In some embodiments, the endonuclease is an RNAse. In some embodiments, the endonuclease is one of RNase A, RNase C, RNase H, and RNase I. In some embodiments, the endonuclease is RNAse H. In some embodiments, the RNase H is RNase H1 or RNase H2. The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864. In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### M. Exemplary Methods of the Disclosure

A particular valuable application of the methods and compositions described herein is the generation of sequences encoding BCR and TCR from a tumor sample of interest. Accordingly, some embodiments of the disclosure relate to methods for generating nucleic acid sequences, e.g., paired, full-length T cell receptor sequences and/or B cell receptor sequences identified from tumor samples. In some embodiments, such methods comprise identifying paired, full-length T cell receptor sequences and/or B cell receptor sequences from a tumor sample. As described in Example 6, the methods can begin by preparing a tumor sample (e.g., comprising fresh frozen dissociated tumor cells), e.g., according to the 10×Genomics protocol “Thawing Dissociated Tumor Cells for Single Cell RNA Sequencing” (protocol #CG000233, publicly available at 10×Genomics Inc. website). The prepared sample can then be partitioned according to a method disclosed herein. For example, the prepared sample can be processed according to the Chromium Single Cell 5′ Reagent Kits User Guide (v2 Chemistry Dual Index) (protocol #CG000331, publicly available at 10×Genomics Inc. website) to generate Gene Expression, TCR Amplified, and/or BCR Amplified sequencing libraries. Data can be analyzed, e.g., using Cell Ranger 6.0, Loupe 5.0, and Enclone. The analysis can identify paired full-length TCR and/or BCR sequences from the tumor samples.

In some embodiments, the methods further comprise production of barcoded recombinant antibodies or TCRs. As described in greater detail in Example 7, by using the BCR sequences identified according to the methods described herein (e.g., as described in Example 6), nucleotide sequences encoding variable heavy chain and light chain domains of antibodies may be reformatted (for example, to IgG1) and synthesized and cloned into a mammalian expression vector. Exemplary mammalian expression vectors are commercially available, e.g., pTwist CMV BG WPRE Neo (Twist Bioscience eCommerce portal), AddGene, InvivoGen, and Human IgG Vector Set from SigmaAldrich. Light chain variable domains may be reformatted into kappa and lambda frameworks accordingly. Clonal genes may be delivered as purified plasmid DNA ready for transfection in human embryonic kidney (HEK) Expi293 cells (Thermo Scientific). Alternatively, ExpiCHO cells may be used for transfection. Cultures may be grown, harvested, and purified using a suitable purification technique such as, Protein A resin (PhyNexus) on the Hamilton Microlab STAR platform to produce a recombinant antibody.

Additionally or alternatively, using the TCR sequences identified according to the methods described herein (e.g., as described in Example 6), nucleotide sequences encoding TCR alpha and TCR beta chains may be synthesized and cloned into a mammalian expression vector. Clonal genes can then be delivered as purified plasmid DNA ready for introduction in cultured cells, e.g., Jurkat cells. Such constructs may be introduced via using classical transformation techniques, e.g., transfection, transduction, or using more precise techniques such as guide RNA (gRNA)-directed CRISPR/Cas genome editing, DNA-guided endonuclease genome editing with NgAgo (Natronobacterium gregoryi Argonaute), or TALENs genome editing (transcription activator-like effector nucleases). Cultures can be grown, harvested, and purified to produce a recombinant TCR. A TCR generally includes two polypeptides (e.g., polypeptide chains), such as a α-chain of a TCR, a β-chain of a TCR, a γ-chain of a TCR, a δ-chain of a TCR, or a combination thereof. Several approaches, techniques, and associated reagents for construction of recombinant TCR are known in the art. In some cases, the TCR constant region may be further alterred to remove one or more domains thereof, which can be achieved by a known genome editing technique (e.g., CRISPR/Cas or TALENs discussed herein), via either homology directed repair, non-homologous end joining (NHEJ), and/or or microhomology-mediated end joining.

In some embodiments, the methods further comprise coupling a reporter oligonucleotide comprising a reporter barcode sequence to the recombinant antibody or TCR. A reporter oligonucleotide comprising a reporter barcode sequence can be coupled to the recombinant antibody or TCR according to available methods. The reporter barcode sequence can be used as an identifier sequence for the antibody or TCR coupled thereto. In some instances, the reporter oligonucleotide is coupled to the recombinant antibody or TCR using chemical conjugation techniques (e.g., Lightning-Link® antibody labelling kits available from Innova Biosciences). In some instances, the reporter oligonucleotide is coupled to the recombinant antibody or TCR using non-covalent attachment mechanisms, e.g., using biotinylated antibodies and oligonucleotides (or beads that include one or more biotinylated linker, coupled to oligonucleotides) with an avidin or a streptavidin linker in monomeric or multimeric form (e.g., tetramic form of streptavidin). Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715. Likewise, protein and peptide biotinylation techniques have been developed and are readily available. See, e.g., U.S. Pat. No. 6,265,552. In some instances, the reporter oligonucleotide may be coupled to the recombinant antibody or TCR using click reaction chemistry such as a Methyltetrazine-PEG5-NHS Ester reaction, a TCO-PEG4-NHS Ester reaction. In some instances, the reporter oligonucleotide may be coupled to the recombinant antibody or TCR using a commercially available kit, such as from Thunderlink or Abcam. In some instances, the reporter oligonucleotide is coupled to the recombinant antibody or TCR indirectly (e.g., via hybridization). In some instances, the recombinant antibody or TCR may be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides may be releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some embodiments, the methods further comprise analysis of the barcoded recombinant antibodies or TCRs. In particular embodiments, the analysis comprises contacting one of more of the barcoded recombinant antibodies or TCRs with a second tumor sample that is a dissociated tumor sample, e.g., a comprising dissociated tumor cells. In some embodiments, the sample is incubated with a cocktail of recombinant barcoded antibodies and/or TCRs. Optionally, the cocktail includes barcoded antibodies for known immune cell markers. Optionally, the cocktail includes barcoded antibodies for known tumor cell markers. Optionally, the cocktail includes one or more barcoded therapeutic antibodies. In some embodiments, the barcoded antibodies (e.g., barcoded antibodies for known immune cell markers, barcoded antibodies for known tumor cell markers, barcoded therapeutic antibodies) are coupled to reporter oligonucleotides comprising reporter barcode sequences that identify the antibody coupled thereto. Methods for contacting one or more of the barcoded recombinant antibodies or TCRs with a second tumor sample (e.g., comprising dissociated tumor cells) are described in further detail in Example 8. In some embodiments, the sample undergoes enrichment, e.g., via sorting as described in further detail, e.g., in Example 8. In some embodiments, the sample is partitioned according to a method disclosed herein. See, e.g., Example 8. In some embodiments, sequencing libraries are generated from the partitioned sample and sequenced according to a method disclosed herein. See, e.g., Example 8. In some embodiments, sequence analysis is used to identify one or more recombinant barcoded antibodies and/or barcoded recombinant TCRs (e.g., as produced according to the methods of Example 7) as having specificity for the tumor. In some instances, comparative analysis of gene expression and the reporter oligonucleotide datasets is performed to determine the recombinant antibodies' specificity and target specificity.

In particular embodiments, the analysis comprises contacting one of more of the barcoded recombinant antibodies or TCRs with a second tumor sample that is an intact tumor sample, e.g., a fresh frozen tissue section comprising tumor tissue. The sample can be mounted on a slide including an array of spatially barcoded capture probes (e.g., a Visium Spatial Gene Expression slide as described in the Visium Spatial Gene Expression Reagent Kits User Guide (e.g., Rev D, dated October 2020). The sample can be subjected to fixation. The sample can be subjected to a blocking step. The sample can be incubated with a cocktail of recombinant barcoded antibodies and/or TCRs. Optionally, the cocktail can include barcoded antibodies for known immune cell markers. Optionally, the cocktail includes barcoded antibodies for known tumor cell markers. Optionally, the cocktail can include one or more barcoded therapeutic antibodies. The barcoded antibodies (e.g., barcoded antibodies for known immune cell markers, barcoded antibodies for known tumor cell markers, barcoded therapeutic antibodies) can be coupled to reporter oligonucleotides comprising reporter barcode sequences that identify the antibody coupled thereto. The sample can be stained (e.g., with H&E) and imaged according to any of the methods described herein. Optionally, if any of the barcoded antibodies include a fluorescence detection agent, the sample may be imaged via immunofluorescence. The sample can be permeabilized, e.g., according to methods described in the Visium Spatial Gene Expression Reagent Kits User Guide (e.g., Rev D, dated October 2020). Transcripts and reporter oligonucleotides can be released during permeabilization for capture onto the spatially barcoded array. The captured transcripts and reporter oligonucleotides can be used in an extension reaction to produce spatially barcoded extension products comprising sequences corresponding to the captured transcripts and/or reporter oligonucleotides, respectively. The spatially barcoded extension products can be used to produce gene expression and reporter oligonucleotide libraries. Sequence analysis can be used to identify one or more recombinant barcoded antibodies and/or barcoded recombinant TCRs (e.g., as produced according to the methods of Example 7) as having specificity for the tumor. Comparative analysis of gene expression, the reporter oligonucleotide, and the image datasets, can be performed to determine the recombinant antibodies' specificity and target specificity.

In particular embodiments, the analysis comprises contacting one of more of the barcoded recombinant antibodies or TCRs with a second tumor sample that is an intact tumor sample, e.g., a formalin-fixed paraffin embedded (FFPE) sample comprising tumor tissue. The sample can be mounted on a slide including an array of spatially barcoded capture probes (e.g., a spatially barcoded array slide as described in the the Visium Spatial Gene Expression for FFPE User Guide (e.g., Rev A, dated June 2021)). The slide-mounted sample can be dried overnight in a desiccator. The sample can be heated to 60° C., followed by deparaffinization and rehydration. H&E staining can be performed and the sample can be imaged. The sample can be destained using a suitable buffer (e.g., HCl and decrosslinked for 1 hour in citrate buffer (pH 6.0) at 95° C.). After decrosslinking, the sample can be incubated overnight with RTL (templated ligation) probe sets at 50° C., e.g., according to methods described in the Visium Spatial Gene Expression for FFPE User Guide (e.g., Rev A, dated June 2021). The sample can be washed to remove un-hybridized probes, then treated with ligase to ligate the RTL probes. The sample can be washed, then blocked with antibody blocking buffer. The sample can be incubated overnight with a cocktail of recombinant barcoded antibodies and/or TCRs. Optionally, the cocktail can include barcoded antibodies for known immune cell markers. Optionally, the cocktail can include barcoded antibodies for known tumor cell markers. Optionally, the cocktail includes one or more barcoded therapeutic antibodies. The barcoded antibodies (e.g., barcoded antibodies for known immune cell markers, barcoded antibodies for known tumor cell markers, barcoded therapeutic antibodies) can be coupled to reporter oligonucleotides comprising reporter barcode sequences that identify the antibody coupled thereto. The sample can be washed with PBST, and washed with SSC. The sample can be subjected to a 30 minute probe release step with RNase, followed by a 1 hour permeabilization step with a permeabilization buffer including Proteinase K and detergent. Accordingly, the ligation products and reporter oligonucleotides of the barcoded antibodies can be captured by the capture probes of the spatially barcoded array slide. The slide can be washed twice (e.g., with 2×SSC) and subjected to probe extension, denaturation, and pre-amplification followed by amplification and sequencing of the templated ligation and reporter oligonucleotide libraries. Sequence analysis can be used to identify one or more recombinant barcoded antibodies and/or barcoded recombinant TCRs (e.g., as produced according to the methods of Example 7) as having specificity for the tumor. Comparative analysis of the templated ligation, the reporter oligonucleotide, and the image datasets can performed to determine the recombinant antibodies' specificity and target specificity.

### Compositions of the Disclosure

As described in greater detail below, one aspect of the present disclosure relates to recombinant antibodies or functional fragments thereof generated or identified by a method disclosed herein. Also provided, in other related aspects of the disclosure, are nucleic acids encoding the recombinant antibodies as disclosed herein or functional fragments thereof, recombinant cells expressing the recombinant antibodies as disclosed herein or functional fragments thereof, pharmaceutical compositions containing the nucleic acids and/or recombinant cells as disclosed herein.

### A. Recombinant Nucleic Acids

In discussed above, one aspect of the disclosure relates to recombinant nucleic acids including a nucleic acid sequence that encode the recombinant antibody of the disclosure or a functional fragment thereof. In some embodiments, the recombinant nucleic acids of the disclosure can be configured as expression cassettes or vectors containing these nucleic acid molecules operably linked to heterologous nucleic acid sequences such as, for example, regulatory sequences which allow in vivo expression of the receptor in a host cell.

Nucleic acid molecules of the present disclosure can be of any length, including for example, between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

Accordingly, in some embodiments, provided herein is a nucleic acid molecule including a nucleotide sequence encoding a recombinant antibody of the disclosure or a functional fragment thereof. In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette can be inserted into a vector for targeting to a desired host cell and/or into an individual. As such, in some embodiments, an expression cassette of the disclosure include a coding sequence for a recombinant antibody of the disclosure or a functional fragment thereof, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that can be used for the purpose of transformation, e.g., the introduction of heterologous DNA into a host cell. As such, in some embodiments, the vector can be a replicon, such as a plasmid, phage, or cosmid, into which another DNA segment can be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector can refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

The nucleic acid sequences encoding the recombinant antibodies as disclosed herein can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the recombinant antibodies disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the recombinant antibodies disclosed herein. The expression cassette generally contains coding sequences and sufficient regulatory information to direct proper transcription and/or translation of the coding sequences in a recipient cell, in vivo and/or ex vivo. The expression cassette can be inserted into a vector for targeting to a desired host cell and/or into an individual. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any recombinant antibody or a functional fragment thereof as disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

For example, a recombinant antibody or a functional fragment thereof as disclosed herein can be produced in a eukaryotic host, such as a mammalian cells (e.g., COS cells, NIH 3T3 cells, or HeLa cells). These cells are available from many sources, including the American Type Culture Collection (Manassas, VA). In selecting an expression system, it matters only that the components are compatible with one another. Artisans or ordinary skill are able to make such a determination. Furthermore, if guidance is required in selecting an expression system, skilled artisans can consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an anti sense strand).

The nucleic acid molecules are not limited to sequences that encode polypeptides (e.g., antibodies); some or all of the non-coding sequences that lie upstream or downstream from a coding sequence (e.g., the coding sequence of an antibody) can also be included. Those of ordinary skill in the art of molecular biology are familiar with routine procedures for isolating nucleic acid molecules. In the event the nucleic acid molecule is a ribonucleic acid (RNA), molecules can be produced, for example, by in vitro transcription.

### B. Recombinant Cells and Cell Culture

The nucleic acid of the present disclosure can be introduced into a host cell, such as, for example, a human T lymphocyte, to produce a recombinant cell containing the nucleic acid molecule. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for transient expression. Accordingly, in some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells can be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the cell is a stem cell. In some embodiments, the cell is a hematopoietic stem cell.

In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a natural killer T (NKT) cell, a basophil, an eosinophil, a neutrophil, a dendritic cell, a macrophage, a regulatory T cell, a helper T cell (T_{H}), a cytotoxic T cell (T_{CTL}), or other T cell. In some embodiments, the immune system cell is a T lymphocyte. In some embodiments, the cell is a precursor T cell or a T regulatory (Treg) cell. In some embodiments, the cell is a CD34+, CD8+, or a CD4+ cell. In some embodiments, the cell is a CD8+ T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+ T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from an individual. In some embodiments, the subject is a human patient.

In another aspect, some embodiments of the disclosure relate to methods for making a recombinant cell, including (a) providing a cell capable of protein expression and (b) contacting the provided cell with a recombinant nucleic acid of the disclosure.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### C. Compositions and Pharmaceutical Compositions

The recombinant antibodies, nucleic acids, recombinant cells, and/or cell cultures of the disclosure can be incorporated into compositions, including pharmaceutical compositions.

In one aspect, as described in greater detail below, the barcoded recombinant antibodies as described herein can be included in compositions suitable for various downstream applications. In general, the compositions of the disclosure can include at least one barcoded recombinant antibody of the disclosure and one or more of the following: (i) one or more barcoded immune-cell marker antibodies and/or functional fragments thereof; (ii) one or more barcoded tumor-cell marker antibodies and functional fragments thereof; (iii) one or more barcoded therapeutic antibodies and functional fragments thereof; and (iv) one or more barcoded recombinant antibodies identified in the present disclosure as having specificity for a tumor sample. In some embodiments, the barcoded antibodies are each coupled to a reporter oligonucleotide including a reporter barcode sequence. In some embodiments, to facilitate downstream analyses, the reporter barcode sequence coupled to a barcoded antibody is distinguishable from coupled to the other barcoded antibodies.

In some embodiments, one or more of the antibodies are monoclonal antibodies. In some embodiments, one or more of the antibodies are polyclonal antibodies. In some embodiments, one or more of the antibodies are multi-specific antibodies (e.g., bispecific antibodies). Functional fragments of the antibodies suitable for the methods described herein can include F(ab) fragments, Fab′ fragments, F(ab′)2 fragments, FIT domains, and Fc domains.

In another aspect, the recombinant antibodies, nucleic acids, recombinant cells, and/or cell cultures of the disclosure can be incorporated into pharmaceutical compositions. Exemplary compositions of the disclosure include pharmaceutical compositions which generally include one or more of the recombinant antibodies, nucleic acids, recombinant cells, and/or cell cultures as described herein and a pharmaceutically acceptable excipient, e.g., carrier.

In one aspect, provided herein are compositions including a pharmaceutically acceptable excipient and one or more of the following: (a) a recombinant antibody of the disclosure; (b) a recombinant nucleic acid of the disclosure; and (c) a recombinant cell of the disclosure.

The pharmaceutical compositions provided herein can be in any form that allows for the composition to be administered to an individual. In some specific embodiments, the pharmaceutical compositions are suitable for human administration. As used herein, the term “pharmaceutically acceptable” means approved by a regulatory agency of the Federal or a state government or listed in the U.S. Pharmacopeia or other generally recognized pharmacopeiae for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual or an animal (some non-limiting examples include a human, or a mammal). In some embodiments, the individual is a human.

The terms “administration” and “administering”, as used herein, refer to the delivery of a bioactive composition or formulation by an administration route comprising, but not limited to, intranasal, transdermal, intravenous, intra-arterial, intramuscular, intraperitoneal, subcutaneous, intramuscular, oral, and topical administration, or combinations thereof. The term includes, but is not limited to, administering by a medical professional and self-administering.

In some embodiments, the pharmaceutical compositions of the present disclosure are formulated to be suitable for the intended route of administration to an individual. For example, the pharmaceutical composition can be formulated to be suitable for parenteral, intraperitoneal, colorectal, intraperitoneal, and intratumoral administration. In some embodiments, the pharmaceutical composition can be formulated for intravenous, oral, intraperitoneal, intratracheal, subcutaneous, intramuscular, topical, or intratumoral administration. One of ordinary skilled in the art will appreciate that the formulation should suit the mode of administration.

For example, pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In some embodiments, the composition should be sterile and should be fluid to the extent that easy syringability exists. It can be stabilized under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

### D. Kits

Also provided herein are kits for the practice of a method described herein. A kit can include instructions for use thereof and one or more of the recombinant antibodies or functional fragments thereof, recombinant nucleic acids, recombinant cells, and compositions as described and provided herein. For examples, some embodiments of the disclosure provide kits that include one or more of the recombinant antibodies described herein and/or functional fragments thereof, and instructions for use. In some embodiments, provided herein are kits that include one or more recombinant nucleic acids, recombinant cells, and compositions as described herein and instructions for use thereof.

In some embodiments, the components of a kit can be in separate containers. In some other embodiments, the components of a kit can be combined in a single container.

In some embodiments, a kit can further include instructions for using the components of the kit to practice a method described herein. The instructions for practicing the method are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### D. Computer Systems

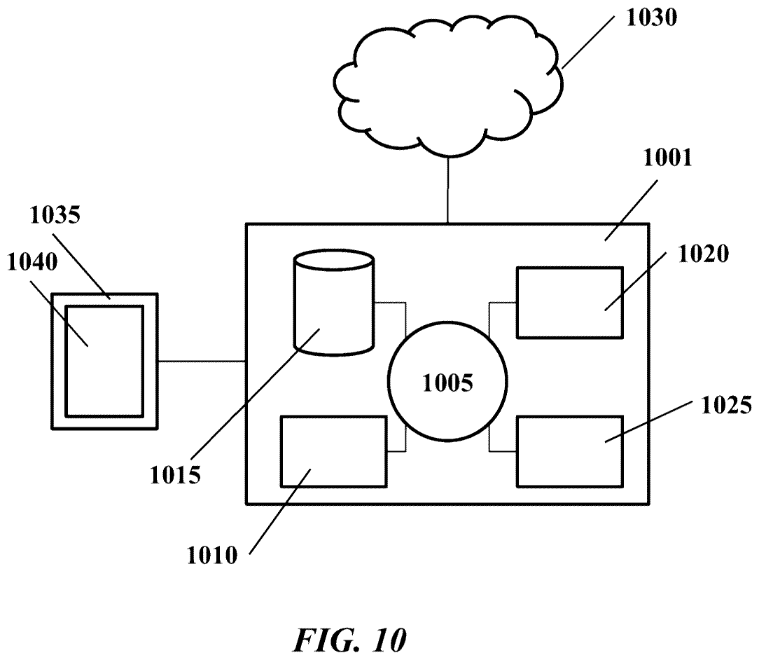

The present disclosure provides computer systems that are programmed to implement methods of the disclosure. FIG. 10 shows a computer system 1001 that is programmed or otherwise configured to (i) control a microfluidics system (e.g., fluid flow), (ii) sort occupied droplets from unoccupied droplets, (iii) polymerize droplets, (iv) partition cell beads or cells into partitions (e.g., droplets or wells), (v) lysate cells and cell beads, (vi) perform sequencing applications, (vii) generate and maintain libraries of cytokine or other analyte specific antibody barcode sequences, MHC multimer barcode sequences, cell surface protein barcode sequences, and cDNAs generated from mRNAs respectively (vi) analyze such libraries. The computer system 1001 can regulate various aspects of the present disclosure, such as, for example, regulating fluid flow rate in one or more channels in a microfluidic structure, regulating polymerization application units, regulating sequence application unit, etc. The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which can enable devices coupled to the computer system 1001 to behave as a client or a server.

The CPU 1005 can execute a sequence of machine-readable instructions, which can be embodied in a program or software. The instructions can be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs.

The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which can provide non-transitory storage at any time for the software programming. All or portions of the software can at times be communicated through the Internet or various other telecommunication networks. Such communications, for example, can enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, can take many forms, including but not limited to, a tangible storage medium, a carrier wave medium or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instructions to a processor for execution.

The computer system 1001 can include or be in communication with an electronic display 1035 that includes a user interface (UI) 1040 for providing, for example, results of sequencing analysis, etc. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, perform nucleotide sequence amplification, sequencing sorting based on barcode sizes, sequencing amplified barcode sequences, analyzing sequencing data, etc.

Devices, systems, compositions and methods of the present disclosure can be used for various applications, such as, for example, processing a single analyte (e.g., RNA, DNA, or protein) or multiple analytes (e.g., DNA and RNA, DNA and protein, RNA and protein, or RNA, DNA and protein) from a single cell. For example, a biological particle (e.g., a cell or cell bead) is partitioned in a partition (e.g., droplet), and multiple analytes from the biological particle are processed for subsequent processing. The multiple analytes can be from the single cell. This can enable, for example, simultaneous proteomic, transcriptomic and genomic analysis of the cell.

### E. Systems

The methods described above for analyzing biological samples can be implemented using a variety of hardware components. In this section, examples of such components are described. However, it should be understood that in general, the various steps and techniques discussed herein can be performed using a variety of different devices and system components, not all of which are expressly set forth.

In another aspect, some embodiments of the disclosure relate to systems for antibody discovery/management, the systems including: (a) a processor, e.g., a CPU, computer processor, or logic processor; (b) a data compiler communicatively coupled to the processor; (c) a stored program code that is executable by the processor; and (d) a report engine communicatively coupled to the processor, wherein reports produced by the report engine depend upon results from execution of the program code, wherein the program code configures the processor to receive from the data compiler information input pertaining to an antibody profile including a preselected set of data input in order to assign a relative performance score to the antibody's tumor specificity based at least in part on the antibody profile, whereby determining the likelihood of the antibody to exhibit one or more tumor specificity attributes as indicated by the assigned relative performance score.

Non-limiting exemplary embodiments of the systems of the disclosure can include one or more of the following features. In some embodiments, the data input includes one or more of the following: (a) antibody sequence data; (b) expression data of biomarkers in the B cell from which the antibody is derived; (c) transcriptomic data for the B cell from which the antibody is derived; and (d) genomic DNA sequence data from whole-exome sequencing. In some embodiments, the systems of the disclosure further include generating an antibody profile report that contains information relevant to the antibody identified as a tumor-specific antibody. In some embodiments, the antibody profile report is characterized as having an encoding selected from the group consisting of “.doc”; “.pdf”; “.xml”; “.html”; “.jpg”; “.aspx”; “.php”, and a combination of any thereof.

In yet another aspect, provided herein is a non-transitory computer readable medium containing machine executable instructions that when executed cause a processor to perform operations including: receiving an antibody profile including a preselected set of data input; assigning, based at least in part on the antibody profile, a relative performance score to the antibody's tumor specificity; and outputting an antibody profile report for the antibody based upon the assigned performance score. Accordingly, antibody profile reports generated by the systems of the disclosure are also with the scope of this disclosure.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

### Example 1

### Characterization of Antibody Specificity and/or Target Specificity

This Example describes experiments performed to characterize antibody specificity and/or target specificity of a given antibody in accordance with some embodiments of the disclosure.

In these experiments, single B cells derived from a tumor sample are individually partitioned to discrete droplets together with hydrogel beads coupled with nucleic acid barcode molecules to generate droplets that contain a single B cell and a single bead. In some embodiments, the tumor sample comprising B cells is partitioned into the discrete droplets. In some embodiments, B cells from the tumor sample are isolated and/or enriched from the tumor sample prior to the partitioning. After cell lysis and reverse transcription of V_{H} and V_{L} mRNAs in the droplets, the complementary DNAs from each cell carry a unique barcode that allows cognate V_{H} and V_{L} pairs to be identified by high-throughput sequencing (e.g., NGS), followed by gene synthesis, cloning, production of selected recombinant antibodies.

For subsequent characterization and validation of the phenotypic properties of the selected recombinant antibodies. The recombinant antibodies produced as described above are coupled a reporter oligonucleotide including a reporter barcode sequence to generate barcoded recombinant antibodies.

The barcoded recombinant antibodies produced as described above are subsequently contacted with a tumor sample to identify those having specificity for the tumor sample as determined by their capability of binding to a tumor cell of the tumor sample and/or an antigen associated with the tumor sample.

Additionally, gene expression and protein marker expression analyses are performed on (1) the tumor sample from which the B cell is derived, and/or (2) the tumor sample from which the V_{H} and V_{L} mRNAs are derived. Comparative analysis of the gene expression and protein marker expression datasets from to (1) and/or (2) is subsequently performed to determine the recombinant antibodies' specificity and target specificity.

### Example 2

### Identification of Patient-Specific or Population-Specific Biomarkers of Cancer

This Example describes experiments performed to identify patient-specific or population-specific biomarkers of cancer in accordance with some embodiments of the disclosure.

In these experiments, a collection of barcoded recombinant antibodies is generated as described in Example 1 above. A tumor sample (e.g., second tumor sample as described herein) taken from a cancer patient (e.g., the same patient that provided the tumor sample in Example 1) is subsequently contacted with a mixture of barcoded antibodies. In some embodiments, a plurality of tumor samples taken from multiple cancer patients suffering from the same cancer type is contacted with the mixture of barcoded antibodies. The mixture of barcoded antibodies can comprise any one of or more of (i) one or more barcoded immune-cell marker antibodies and/or functional fragments thereof; (ii) one or more barcoded tumor-cell marker antibodies and functional fragments thereof; (iii) one or more barcoded therapeutic antibodies and functional fragments thereof; and (iv) one or more barcoded recombinant antibodies identified and produced according to Example 1. Comparative analysis of in vitro and/or in vivo characterization the barcoded recombinant antibodies as well as gene expression and protein marker expression analysis of the tumor samples are performed to identify antibodies specific for a patient or a population of patients.

Additionally, comparative analysis of in vitro and/or in vivo characterization the barcoded recombinant antibodies as well as gene expression and protein marker expression analysis of a population of tumor samples are performed to identify biomarkers specific for individual tumor sample or for a population of tumor samples. In some embodiments, sequencing analysis of barcode sequences corresponding to (i) the one or more barcoded immune-cell marker antibodies and/or functional fragments thereof; and/or (ii) the one or more barcoded tumor-cell marker antibodies and functional fragments thereof, is used to identify patient-specific or population-specific biomarkers for the tumor or cancer.

### Example 3

### Monitoring Antigen Escape

This Example describes experiments performed to monitor antigen escape in an individual who has been treated with an antibody-based therapy in accordance with some embodiments of the disclosure.

In these experiments, a barcoded recombinant antibody having specificity for a tumor sample is generated as described in Example 1 above. The binding affinity of the barcoded recombinant antibody to a second tumor sample is subsequently evaluated by measuring the number of tumor cells expressing a target antigen of the barcoded recombinant antibody that are capable to binding to the barcoded recombinant antibody. In these experiments, the quantified binding affinity of the barcoded recombinant antibody to the second tumor sample is indicative of the recombinant antibody's efficacy in treating the tumor.

Additionally, the binding affinity of the barcoded recombinant antibody to an antigen expressed by the tumor sample is monitored over time, and is used as an indication of antigen escape from the recombinant antibody over time.

### Example 4

### Characterization of Potential Antigens

This Example describes experiments performed to identify and characterize potential antigens in accordance with some embodiments of the disclosure.

In these experiments, a collection of barcoded recombinant antibodies is generated as described in Example 1 above. The binding affinity of the barcoded recombinant antibodies to a second tumor sample is subsequently evaluated by quantifying binding affinity of the barcoded therapeutic antibodies to the second tumor sample. This is accomplished by measuring the number of tumor cells that express at least one antigen that binds to the one or more barcoded therapeutic antibodies. The quantified binding affinity is then used to determine if the recombinant antibodies compete with one another for binding to the second tumor sample. In addition, the quantified binding affinity of the recombinant antibodies is also used to co-associate with RNA expression analysis in identifying potential antigens capable of binding to the tested recombinant antibodies.

### Example 5

### Generation and Characterization of Antibodies from Melanoma Samples

This Example describes the results of experiments performed to generate and characterize antibodies produced in tumor samples collected from melanoma patients.

In these experiments, three samples were obtained from three melanoma patients, and were determined to contain multiple immune cell types, including B cells and T cells.

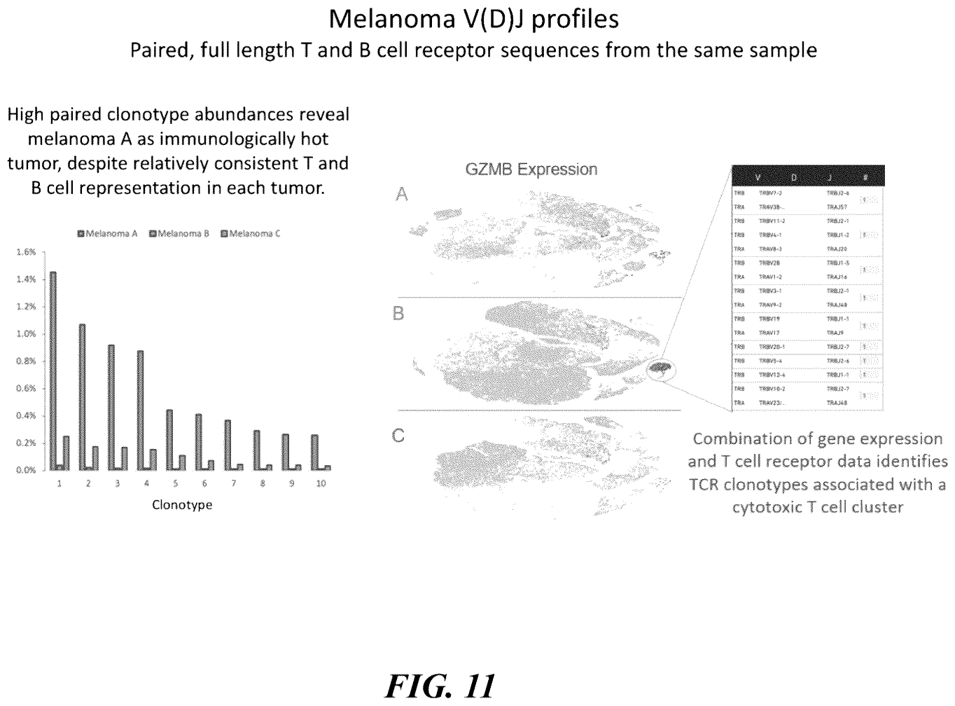

Experiments were performed to illustrate that the compositions and methods of the present disclosure can be used to generate paired, full-length T cell receptor sequences and B cell receptor sequences from tumor samples. In these experiments, the T and B cell receptor sequences were generated from the same input material as each other and as the gene expression libraries, which allows maximizing the information available from a single experiment. As illustrated in FIG. 11, melanoma V(D)J profiles were generated to demonstrated that paired, full-length T and B cell receptor sequences could be identified and obtained from all 3 melanoma samples. Paired clonotype abundance for the top 10 immunoglobulin sequences for each sample are plotted in Left panel. In these experiments, granzyme B (GZMB) expression was used as a marker for a cytotoxic T cell population in melanoma B, which allows for identification of specific T cell receptor clonotypes associated with specific cell population (FIG. 11, right panel).

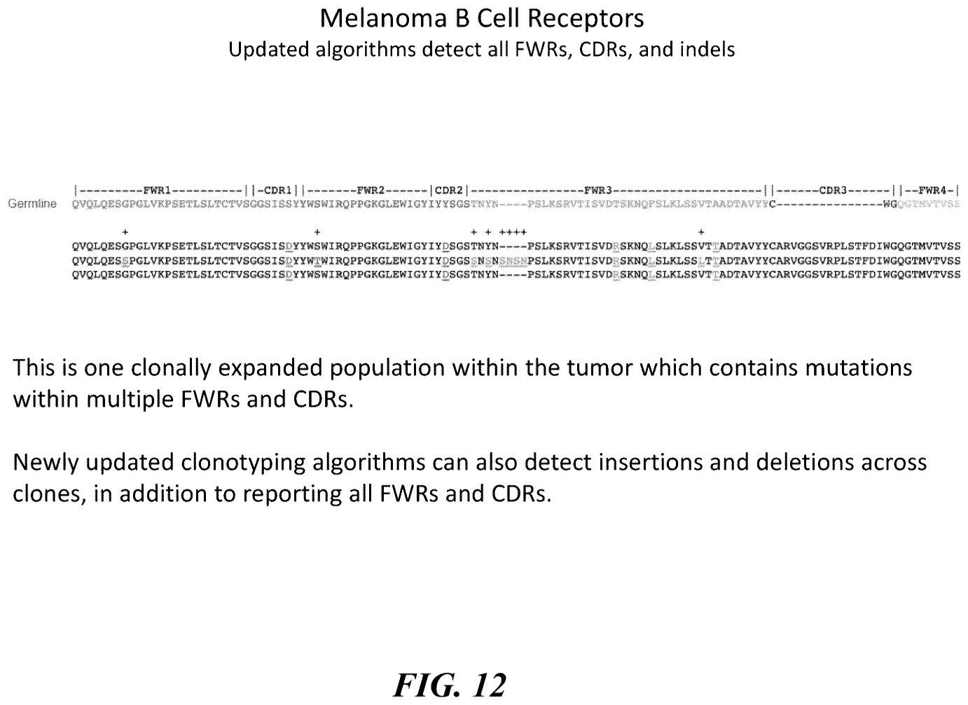

In FIG. 12, a single B cell heavy chain from the melanoma B dataset is shown with a single germline clonotype (top line) along with 3 of its subclonotypes (these are clonotypes belonging to the same B cell lineage based on sequence similarity).

FIG. 12 also shows framework regions (FWRs), complementarity determining regions CDRs, as well as somatic mutations (e.g., amino acid substitutions and indels) within these regions, which can include mutations common to all subclonotypes, as well as variants specific to a single subclonotype. For example, a G→S substitution was found in the FWR1 region of B cell receptor sequences derived from one of the three samples. Additional amino acid substitutions and insertions were also observed in the FWR3 region of B cell receptor sequences obtained from the tumor samples.

Melanoma Samples and Data Analysis

Fresh frozen dissociated tumor cells from 3 different melanomas were obtained from Discovery Life Sciences. These cells were prepared according to 10× Genomics protocol “Thawing Dissociated Tumor Cells for Single Cell RNA Sequencing” (protocol #CG000233, publicly available at 10× Genomics Inc. website). Four replicates of each sample type were run according to Chromium Single Cell 5′ Reagent Kits User Guide (v2 Chemistry Dual Index) (protocol #CG000331, publicly available at 10×Genomics Inc. website) to generate Gene Expression, TCR Amplified, and BCR Amplified sequencing libraries. Data was analyzed using Cell Ranger 6.0, Loupe 5.0, and Enclone.

### Example 6

### Identification of BCR and/or TCR Sequences from First Tumor Samples

Experiments are performed to generate paired, full-length T cell receptor sequences and/or B cell receptor sequences from tumor samples. A first tumor sample (e.g., comprising fresh frozen dissociated tumor cells) are prepared according to 10× Genomics protocol “Thawing Dissociated Tumor Cells for Single Cell RNA Sequencing” (protocol #CG000233, publicly available at 10×Genomics Inc. website). The prepared samples are run according to Chromium Single Cell 5′ Reagent Kits User Guide (v2 Chemistry Dual Index) (protocol #CG000331, publicly available at 10× Genomics Inc. website) to generate Gene Expression, TCR Amplified, and/or BCR Amplified sequencing libraries. Data is analyzed using Cell Ranger 6.0, Loupe 5.0, and Enclone. The analysis identifies paired full-length TCR and/or BCR sequences from the tumor samples.

### Example 7

### Production of Barcoded Recombinant Antibodies or TCRs

Using the BCR sequences identified from Example 6, nucleotide sequences encoding variable heavy chain and light chain domains of antibodies are reformatted to IgG1 and synthesized and cloned into a mammalian expression vector. Exemplary mammalian expression vectors are commercially available, e.g., pTwist CMV BG WPRE Neo (Twist Bioscience eCommerce portal), AddGene, InvivoGen, and Human IgG Vector Set from SigmaAldrich. Light chain variable domains are reformatted into kappa and lambda frameworks accordingly. Clonal genes are delivered as purified plasmid DNA ready for transfection in human embryonic kidney (HEK) Expi293 cells (Thermo Scientific). Alternatively, ExpiCHO cells may be used for transfection. Cultures in a volume of 1.2 ml are grown to four days, harvested, and purified using Protein A resin (PhyNexus) on the Hamilton Microlab STAR platform into 43 mM Citrate 148 mM HEPES, pH 6 to produce a recombinant antibody.

Alternatively, using the TCR sequences identified from Example 6, nucleotide sequences encoding TCR alpha and TCR beta chains are synthesized and cloned into a mammalian expression vector. Clonal genes are delivered as purified plasmid DNA ready for introduction in cultured cells, e.g., Jurkat cells. Such constructs may be introduced via using classical transformation techniques, e.g., transfection, transduction, or using more precise techniques such as guide RNA (gRNA)-directed CRISPR/Cas genome editing, DNA-guided endonuclease genome editing with NgAgo (Natronobacterium gregoryi Argonaute), or TALENs genome editing (transcription activator-like effector nucleases). Cultures are grown, harvested, and purified to produce a recombinant TCR. A TCR generally includes two polypeptides (e.g., polypeptide chains), such as a α-chain of a TCR, a β-chain of a TCR, a γ-chain of a TCR, a δ-chain of a TCR, or a combination thereof. Several approaches, techniques, and associated reagents for construction of recombinant TCR are known in the art. In some embodiments, the TCR constant region may be further alterred to remove one or more domains thereof, which can be achieved by a known genome editing technique (e.g., CRISPR/Cas or TALENs discussed herein), via either homology directed repair, non-homologous end joining (NHEJ), and/or or microhomology-mediated end joining.

A reporter oligonucleotide comprising a reporter barcode sequence is coupled to the recombinant antibody or TCR according to available methods. The reporter barcode sequence is used as an identifier sequence for the antibody or TCR coupled thereto. In some instances, the reporter oligonucleotide is coupled to the recombinant antibody or TCR using chemical conjugation techniques (e.g., Lightning-Link® antibody labelling kits available from Innova Biosciences). In some instances, the reporter oligonucleotide is coupled to the recombinant antibody or TCR using non-covalent attachment mechanisms, e.g., using biotinylated antibodies and oligonucleotides (or beads that include one or more biotinylated linker, coupled to oligonucleotides) with an avidin or an streptavidin linker in monomeric or multimeric form (e.g., tetramic form of streptavidin). Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. In some instances, the reporter oligonucleotide is coupled to the recombinant antibody or TCR using click reaction chemistry such as a Methyltetrazine-PEG5-NHS Ester reaction, a TCO-PEG4-NHS Ester reaction. In some instances, the reporter oligonucleotide is coupled to the recombinant antibody or TCR using a commercially available kit, such as from Thunderlink or Abcam. In some instances, the reporter oligonucleotide is coupled to the recombinant antibody or TCR indirectly (e.g., via hybridization). In some instances, the recombinant antibody or TCR is directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

### Example 8

### Analysis of Barcoded Recombinant Antibodies and/or TCRs Using Single Cell Methods

Recombinant barcoded antibodies and/or TCRs (e.g., produced according to methods described in Example 7), are further analyzed as follows.

A second tumor sample (e.g., comprising fresh frozen dissociated tumor cells) is prepared according to 10× Genomics protocol “Thawing Dissociated Tumor Cells for Single Cell RNA Sequencing” (protocol #CG000233, publicly available at 10× Genomics Inc. website). Once thawed, the sample is incubated with a cocktail of recombinant barcoded antibodies and/or TCRs. Optionally, the cocktail includes barcoded antibodies for known immune cell markers. Exemplary antibodies for immune cell markers include the following TotalSeq-C oligo barcoded antibodies: TotalSeq-00389 anti-human CD38, Total Seq-00154 anti-human CD27, TotalSeq-00189 anti-human CD24, TotalSeq-00384 anti-human IgD, TotalSeq-00100 anti-human CD20, TotalSeq-00050 anti-human CD19 (clone HIB19, to distinguish it from the flow clone), TotalSeq-00049 anti-human CD3E, TotalSeq-00045 anti-human CD4, TotalSeq-00046 anti-human CD8A, TotalSeq-00051 anti-human CD14, TotalSeq-00083 anti-human CD16, TotalSeq-00090 mouse IgG1 K isotype control, TotalSeq-00091 mouse IgG2a K isotype control. Optionally, the cocktail includes barcoded antibodies for known tumor cell markers. Optionally, the cocktail includes one or more barcoded therapeutic antibodies. It is to be understood that the barcoded antibodies (e.g., barcoded antibodies for known immune cell markers, barcoded antibodies for known tumor cell markers, barcoded therapeutic antibodies) are coupled to reporter oligonucleotides comprising reporter barcode sequences that identify the antibody coupled thereto.

Cells are stained in labeling buffer (1% BSA in PBS) in the dark for 30 minutes on ice, then cells are washed 3 times with 2 mL of cold labeling buffer at 350*g for 5 minutes at 4° C., resuspended in cold labeling buffer and a 1:200 addition of live/dead cell discriminating agent 7AAD for 10 minutes on ice in the dark, then washed one more time with labeling buffer at 350*g for 5 minutes at 4 C, then resuspended in labeling buffer and loaded into a Sony MA900 Cell Sorter using a 70 microM sorting chip.

Cells are gated on being single, live (7AAD^{negative}). Optionally, if any of the barcoded antibodies include a fluorescent detection agent (e.g., PE, APC), cells are gated based on fluorescent status directedly into master mix and water.

The resulting volume is adjusted with additional water to match the recommended volume and target for loading with the 10× 5′V2 Single Cell Immune Profiling kit. FACS data is analyzed using FlowJo. Standard gene expression, V(D)J, and reporter oligonucleotide libraries are constructed using the 10× 5′V2 Single Cell Immune Profiling kit per manufacturer's instructions. Additional information in this regard can be found at “support.10×genomics.com/permalink/getting-started-immune-profiling-feature-barcoding.” Alternatively, the resulting volume can be adjusted to match the recommended volume and target for loading with the 10× 3′ V3 Single Cell reagent kit. Gene expression and reporter oligonucleotide libraries can be constructed using the 10× 3′ V3 Single Cell reagent kit per manufacturer's instructions.

The libraries are sequenced on a NovaSeq 3 using a NovaSeq S4 200 cycles 2020 v1.5 kit, targeting using read 28, 10, 10, and 90 cycles targeting 20,000, 30,000, or 6000 reads per cell for gene expression, reporter oligonucleotide, or Ig libraries, respectively.

Sequence analysis is used to identify one or more recombinant barcoded antibodies and/or barcoded recombinant TCRs (e.g., as produced according to the methods of Example 7) as having specificity for the tumor. In some instances, comparative analysis of gene expression and the reporter oligonucleotide datasets is performed to determine the recombinant antibodies' specificity and target specificity.

### Example 9

### Analysis of Barcoded Recombinant Antibodies and/or TCRs Using Spatial Analysis Methodologies for Fresh Frozen Tumor Tissue Samples

Recombinant barcoded antibodies and/or TCRs (e.g., produced according to methods described in Example 7), are further analyzed as follows.

A second tumor sample (e.g., a fresh frozen tissue section comprising tumor tissue) is mounted on a slide including an array of spatially barcoded capture probes (e.g., a Visium Spatial Gene Expression slide as described in the Visium Spatial Gene Expression Reagent Kits User Guide (e.g., Rev D, dated October 2020). After fixation (e.g., with 2% formalin, or with methanol) and blocking (e.g., with Triton-X), the second tumor sample is incubated with a cocktail of recombinant barcoded antibodies and/or TCRs. Optionally, the cocktail includes barcoded antibodies for known immune cell markers. Optionally, the cocktail includes barcoded antibodies for known tumor cell markers. Optionally, the cocktail includes one or more barcoded therapeutic antibodies. It is to be understood that the barcoded antibodies (e.g., barcoded antibodies for known immune cell markers, barcoded antibodies for known tumor cell markers, barcoded therapeutic antibodies) are coupled to reporter oligonucleotides comprising reporter barcode sequences that identify the antibody coupled thereto.

The sample is then stained (e.g., with H&E) and imaged according to any of the methods described herein. Optionally, if any of the barcoded antibodies include a fluorescence detection agent, the sample may be imaged via immunofluorescence.

The sample is permeabilized, e.g., according to methods described in the Visium Spatial Gene Expression Reagent Kits User Guide (e.g., Rev D, dated October 2020). Transcripts and reporter oligonucleotides are released during permeabilization for capture onto the spatially barcoded array. The captured transcripts and reporter oligonucleotides are used in an extension reaction to produce spatially barcoded extension products comprising sequences corresponding to the captured transcripts and/or reporter oligonucleotides, respectively. The spatially barcoded extension products are used to produce gene expression and reporter oligonucleotide libraries.

Sequence analysis is used to identify one or more recombinant barcoded antibodies and/or barcoded recombinant TCRs (e.g., as produced according to the methods of Example 7) as having specificity for the tumor. In some instances, comparative analysis of gene expression, the reporter oligonucleotide, and the image datasets, is performed to determine the recombinant antibodies' specificity and target specificity.

### Example 10

### Analysis of Barcoded Recombinant Antibodies and/or TCRs Using Spatia Analysis Methodologies for FFPE Tumor Tissue Samples

Recombinant barcoded antibodies and/or TCRs (e.g., produced according to methods described in Example 7), are further analyzed as follows.

A second tumor sample (e.g., a formalin-fixed paraffin embedded (FFPE) sample comprising tumor tissue) is mounted on a slide including an array of spatially barcoded capture probes (e.g., a spatially barcoded array slide as described in the the Visium Spatial Gene Expression for FFPE User Guide (e.g., Rev A, dated June 2021)). The slide-mounted sample is dried overnight in a desiccator. The following day, the sample is heated to 60° C., followed by deparaffinization and rehydration. H&E staining is performed and the sample is imaged. The sample is destained using HCl and decrosslinked for 1 hour in citrate buffer (pH 6.0) at 95° C. After decrosslinking, the sample is incubated overnight with RTL (templated ligation) probe sets at 50° C., e.g., according to methods described in the Visium Spatial Gene Expression for FFPE User Guide (e.g., Rev A, dated June 2021). The following day, the sample is washed to remove un-hybridized probes, then treated with ligase to ligate the RTL probes. After another wash step, the sample is blocked with antibody blocking buffer. The sample is incubated overnight with a cocktail of recombinant barcoded antibodies and/or TCRs. Optionally, the cocktail includes barcoded antibodies for known immune cell markers. Optionally, the cocktail includes barcoded antibodies for known tumor cell markers. Optionally, the cocktail includes one or more barcoded therapeutic antibodies. It is to be understood that the barcoded antibodies (e.g., barcoded antibodies for known immune cell markers, barcoded antibodies for known tumor cell markers, barcoded therapeutic antibodies) are coupled to reporter oligonucleotides comprising reporter barcode sequences that identify the antibody coupled thereto.

The sample is washed with PB ST, and washed with SSC. The sample is subjected to a 30 minute probe release step with RNase, followed by a 1 hour permeabilization step with a permeabilization buffer including Proteinase K and detergent. Accordingly, the ligation products and reporter oligonucleotides of the barcoded antibodies are captured by the capture probes of the spatially barcoded array slide. The slide is washed twice with 2×SSC and subjected to probe extension, denaturation, and pre-amplification followed by amplification and sequencing of the templated ligation and reporter oligonucleotide libraries.

Sequence analysis is used to identify one or more recombinant barcoded antibodies and/or barcoded recombinant TCRs (e.g., as produced according to the methods of Example 7) as having specificity for the tumor. In some instances, comparative analysis of the templated ligation, the reporter oligonucleotide, and the image datasets, is performed to determine the recombinant antibodies' specificity and target specificity.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A method for identifying a tumor-specific antibody, the method comprising:
a) partitioning a single B cell of a plurality of single B cells obtained from a first tumor sample in a partition of a first plurality of partitions;
b) determining all or a part of the nucleic acid sequences encoding one or more antibodies produced by the partitioned single B cell;
c) using the determined nucleic acid sequences to produce a recombinant antibody;
d) coupling the recombinant antibody to a reporter oligonucleotide comprising a reporter barcode sequence to generate a barcoded recombinant antibody; and
e) contacting the barcoded recombinant antibody with a second tumor sample, and identifying the recombinant antibody as an antibody having specificity for the second tumor sample if the barcoded recombinant antibody is capable of binding to an antigen associated with the second tumor sample.

2. The method of claim 1, further comprising determining all or a part of the nucleic acid sequence of the reporter oligonucleotide to identify the barcoded recombinant antibody.

3. The method of claim 1, wherein (a) further comprises partitioning one or more nucleic acid barcode molecules into the partition; the one or more nucleic acid barcode molecules comprising a common barcode sequence.

4. The method of claim 3, further comprising using the one or more nucleic acid barcode molecules and one or more nucleic acid analytes of the partitioned B single cell to generate one or more barcoded nucleic acid molecule comprising a coding sequence for an antibody produced by the partitioned B single cell, or any fragments thereof.

5. The method of claim 1, wherein the first and the second tumor samples are derived from (i) the same subject or (ii) the same tumor.

6. The method of claim 1, wherein step (e) further comprises contacting the barcoded recombinant antibody with a control sample.

7. The method of claim 1, wherein step (a) comprising individually partitioning additional single B cells of the plurality of B cells in partitions of the first plurality of partitions, and step (b) further comprising determining all or a part of the nucleic acid sequences encoding antibodies produced by the additional single B cells.

8. The method of claim 1, further comprising individually partitioning one or more single tumor cells from the second tumor sample in a partition of a second plurality of partition.

9. The method of claim 1, wherein the method comprising contacting the single tumor cell obtained from the second tumor sample with a composition comprising one or more of the following:
i) one or more barcoded immune-cell marker antibodies and/or barcoded tumor-cell marker antibodies;
ii) one or more barcoded therapeutic antibodies; and
iii) the barcoded recombinant antibody identified as having specificity for the second tumor sample.

10. The method of claim 1, further comprising comparing the determined nucleic acid sequence of the recombinant antibody to sequences of known antibodies in order to identify the antibody as a tumor-specific antibody.

11. A recombinant antibody or a functional fragment thereof generated or identified by a method according to claim 1.

12. A recombinant nucleic acid comprising a nucleic acid sequence that encode the recombinant antibody of claim 11 or a functional fragment thereof.

13. A recombinant cell comprising a recombinant nucleic acid according to claim 12.

14. A composition comprising one or more of the following:
a) a recombinant antibody of claim 11;
b) a recombinant nucleic acid according to claim 12;
c) a recombinant cell according to claim 13;
d) one or more barcoded immune-cell marker antibodies and/or barcoded tumor-cell marker antibodies;
e) one or more barcoded therapeutic antibodies; and
f) the barcoded recombinant antibody identified in claim 11 as having specificity for the second tumor sample.

15. The method of claim 1, wherein the method further comprising, after step (e):
f) analyzing RNA expression and protein marker expression for the first and/or second tumor samples to determine the recombinant antibody specificity and target specificity.

16. The method of claim 1, wherein the method further comprising, after step (e):
(f) analyzing RNA expression and protein marker expression for the second tumor sample to identify one or more biomarkers specific for the second tumor sample or for a population of tumor samples

17. The method of claim 1, wherein the first and/or the second tumor are obtained from an individual who has been treated with an antibody-based therapy, and wherein the method comprising, after step (e):
f) quantifying binding affinity of a barcoded therapeutic antibody to the second tumor sample, wherein the quantified binding affinity is indicative of the therapeutic antibody's efficacy in treating the tumor; and
g) optionally using the quantified binding affinity to monitor antigen escape from the therapeutic antibody over time.

18. The method of claim 1, wherein the method comprising, after step (e):
f) quantifying binding affinity of the one or more antibodies to the second tumor sample, and using the quantified binding affinity to determine if the one or more antibodies compete with one another for binding to the second tumor sample; and
g) optionally co-associating the quantified binding affinity with RNA expression analysis to identify potential antigen.

19. A system for antibody discovery/management, comprising:
a logic processor; and
a stored program code that is executable by the logic processor, wherein the program code configures the logic processor to receive information input pertaining to an antibody profile comprising a preselected set of data input in order to assign a relative performance score to the antibody's tumor specificity based at least in part on the antibody profile, whereby determining the likelihood of the antibody to exhibit one or more tumor specificity attributes as indicated by the assigned relative performance score.

20. A non-transitory computer readable medium containing machine executable instructions that when executed cause a processor to perform operations comprising:
receiving an antibody profile comprising a preselected set of data input;
assigning, based at least in part on the antibody profile, a relative performance score to the antibody's tumor specificity; and
outputting an antibody profile report for the antibody based upon the assigned performance score.
